(19) United States
(12) Patent Application Publication
(10) Pub. No.: US 2025/0163401 A1
(43) Pub. Date: May 22, 2025

(54) ENGINEERED RNA LIGASES

(71) Applicant: Codexis, Inc., Redwood City, CA (US)

(72) Inventors: Marina Mikhailovna Bakhtina, Redwood City, CA (US); David Elgart, San Mateo, CA (US); Nikki D. Kruse, San Carlos, CA (US); Mathew G. Miller, San Carlos, CA (US); Jovana Nazor, Milpitas, CA (US); Scott J. Novick, Palo Alto, CA (US); Amani Shoubber, Santa Clara, CA (US); Ljubica Vojcic, San Jose, CA (US)

(21) Appl. No.: 18/955,904

(22) Filed: Nov. 21, 2024

Related U.S. Application Data
(60) Provisional application No. 63/601,699, filed on Nov. 21, 2023.

(51) Int. Cl. C12N 9/00; C12N 15/11; C12N 15/70; C12P 21/02

(52) U.S. Cl. CPC C12N 9/93; C12N 15/11; C12N 15/70; C12P 21/02; C12N 2310/315; C12N 2310/32; C12Y 605/01003

(57) ABSTRACT

The present disclosure provides engineered RNA ligases, recombinant polynucleotides encoding the engineered RNA ligases, and compositions of the engineered RNA ligases. The present disclosure further provides uses of the engineered RNA ligases for ligation of polynucleotide substrates.

### CROSS-REFERENCE TO RELATED APPLICATIONS

This application claims the benefit of U.S. Provisional Application 63/601,699, filed Nov. 21, 2023, which is incorporated by reference herein.

### REFERENCE TO SEQUENCE LISTING, TABLE OR COMPUTER PROGRAM

The Sequence Listing concurrently submitted herewith as file name CX9-256WO2_ST26.xml, created on Nov. 13, 2024, with a file size of 1,902,579 bytes, is part of the specification and is hereby incorporated by reference herein.

### BACKGROUND

RNA ligases are a family of enzymes that catalyze the joining of pieces of RNA or DNA that are adjacent to each other. They are involved in the editing and repair of RNA, and therefore, are essential proteins for biological processes. RNA ligases find uses in the profiling of target sequences, the quantification of different RNA species, the detection of particular RNA mutations, and the synthesis of polynucleotides. Based on their substrate preference, the known RNA ligases are categorized into two groups. Single stranded RNA ligases, also referred to as RNA ligase 1 or RNA ligase I, are capable of joining two RNA or DNA without a hybridizing template. Double stranded RNA ligases, also referred to as RNA ligase 2 or RNA ligase II, preferentially join a nick in an RNA duplex. RNA ligase 1 type proteins have been identified in fungi, baculoviruses, archea, and archeal viruses (thermostable properties). RNA ligase 2 type enzymes have been identified in Vibrio phage KVP40, baculoviruses and entomopoxviruses, some parasites, and archaea species.

Protopypical RNA ligases are those from bacteriophage T4. The T4 RNA Ligase 1 ligates a 5′-phosphoryl nucleic acid donor to a 3′-hydroxyl nucleic acid acceptor by connecting them with a phosphodiester bond in an ATP dependent reaction. Substrates for T4 RNA Ligase 1 include ssRNA, ssDNA, and dinucleoside pyrophosphates. T4 RNA ligase 1 is used for ligation of ssRNA and DNA, RNA ligase-mediated rapid amplification of cDNA ends (RLM-RACE), ligation of oligonucleotide adapters to cDNA or single-stranded primer extension products for PCR, oligonucleotide synthesis, and various 5′ nucleotide modifications of nucleic acids. T4 RNA ligase 2 ligates adjacent 5′ phosphate at the end of RNA and DNA strands to 3′ OH of RNA strands in the context of nicked dsRNA or RNA/DNA hybrids. T4 RNA ligase 2 exhibits significant homology to DNA ligases and mRNA capping enzymes, and is used to seal nicks in dsRNA and dsRNA/DNA hybrids. While T4 RNA ligases can act on RNA and DNA, RNA is the preferred substrate, with DNA being a less effective acceptor in the ligation reaction.

While T4 RNA ligases have become useful tools for labeling, circularizing, and ligating RNA and RNA/DNA, desirable are ligases that provide more facile and efficient ligation of polynucleotide substrates, including improved ligation of polynucleotides that contain nucleotide analogs, such as modified sugar residues and non-standard internucleoside linkages.

### SUMMARY

The present disclosure provides engineered RNA ligase polypeptides and compositions thereof, as well as polynucleotides encoding the engineered RNA ligase polypeptides. The present disclosure also provides methods of using the engineered RNA ligase polypeptides and compositions thereof for nucleic acid synthesis, diagnostic assays, and other purposes.

In one aspect, the present disclosure provides an engineered RNA ligase, or functional fragment thereof, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of an even numbered SEQ ID NO. of SEQ ID NOs: 2-220, 224-252, and 270-958, or to a reference sequence corresponding to an even numbered SEQ ID NO. of SEQ ID NOs: 2-220, 224-252, and 270-958, wherein the amino acid sequence comprises one or more substitutions relative to the reference sequence corresponding to residues 12 to 343 of SEQ ID NO: 2, 4, 106, 218, 286, 396, 436, 520, 552, or 582, or relative to the reference sequence corresponding to SEQ ID NO: 2, 4, 106, 218, 286, 396, 436, 520, 552, or 582.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of SEQ ID NO: 2, 4, 106, 218, 286, 396, 436, 520, 552, or 582, or to a reference sequence corresponding to SEQ ID NO: 2, 4, 106, 218, 286, 396, 436, 520, 552, or 582, wherein the amino acid sequence comprises one or more substitutions relative to the reference sequence corresponding to residues 12 to 343 of SEQ ID NO: 2, 4, 106, 218, 286, 396, 436, 520, 552, or 582, or relative to the reference sequence corresponding to SEQ ID NO: 2, 4, 106, 218, 286, 396, 436, 520, 552, or 582.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 343 of SEQ ID NO: 2, or to the reference sequence corresponding to SEQ ID NO: 2, wherein the amino acid sequence comprises one or more substitutions relative to the reference sequence corresponding to residues 12 to 343 of SEQ ID NO: 2, or relative to the reference sequence corresponding to SEQ ID NO: 2.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 343 of SEQ ID NO: 4, 106, 218, 286, 396, 436, 520, 552, or 582, or to the reference sequence corresponding to SEQ ID NO: 4, 106, 218, 286, 396, 436, 520, 552, or 582, wherein the amino acid sequence comprises one or more substitutions relative to the reference sequence corresponding to residues 12 to 343 of SEQ ID NO: 2, or relative to the reference sequence corresponding to SEQ ID NO: 2.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 343 of an even-numbered SEQ ID NO. of SEQ ID NOs: 4-220, 224-252, and 270-958, or to a reference sequence corresponding to an even-numbered SEQ ID NO. of SEQ ID NOs: 4-220, 224-252, and 270-958, wherein the amino acid sequence comprises one or more substitutions relative to the reference sequence corresponding to residues 12 to 343 of SEQ ID NO: 2, or relative to the reference sequence corresponding to SEQ ID NO: 2.

In some embodiments, amino acid sequence of the engineered RNA ligase comprises at least a substitution at amino acid position 11, 12, 14, 16, 17, 23, 25, 26, 30, 32, 33, 38, 40, 52, 57, 60, 68, 69, 71, 73, 75, 79, 82, 83, 92, 93, 95, 96, 98, 100, 101, 102, 103, 111, 114, 117, 118, 119, 120, 135, 136, 141, 142, 145, 147, 156, 167, 168, 170, 171, 174, 177, 179, 183, 184, 185, 188, 189, 190, 191, 192, 193, 196, 200, 202, 205, 206, 207, 209, 221, 229, 254, 255, 259, 270, 271, 287, 288, 289, 292, 295, 296, 300, 307, 320, 327, 332, 333, 334, 335, 336, 337, 339, 342, or 343, or combinations thereof, wherein the amino acid positions are relative to the reference sequence corresponding to residues 12 to 343 of SEQ ID NO: 2, or relative to the reference sequence corresponding to SEQ ID NO: 2.

In some embodiments, the amino acid sequence of the engineered RNA ligase comprises at least a substitution at amino acid position 33, 38, 71, 73, 75, 96, 98, 101, 114, 117, 136, 156, 179, 184, 191, 196, 221, 289, 292, 296, 320, 335, 336, or 339, or combinations thereof, wherein the amino acid positions are relative to the reference sequence corresponding to residues 12 to 343 of SEQ ID NO: 2, or relative to the reference sequence corresponding to SEQ ID NO: 2.

In some embodiments, the amino acid sequence of the engineered RNA ligase comprises at least a substitution or substitution set at amino acid position(s) 179, 33/101/221/320/335/336/339, 38/71/114/184/191/196, 117, 292, 75/136/296, or 98, wherein the amino acid positions are relative to the reference sequence corresponding to residues 12 to 343 of SEQ ID NO: 2, or relative to the reference sequence corresponding to SEQ ID NO: 2.

In some embodiments, the amino acid sequence of the engineered RNA ligase comprises at least a substitution or substitution set at amino acid position 14, 32, 33, 38, 40, 57, 60, 71, 75, 82, 92, 96, 98, 101, 114, 141, 142, 145, 170, 171, 174, 179, 184, 191, 193, 196, 202, 205, 207, 209, 221, 255, 320, 327, 339, or 342, or combinations thereof, wherein the amino acid positions are relative to the reference sequence corresponding to residues 12 to 343 of SEQ ID NO: 2, or relative to the reference sequence corresponding to SEQ ID NO: 2.

In some embodiments, the amino acid sequence of the engineered RNA ligases comprises at least one substitution set forth in Tables 3, 4, 5, 6, 7.1, 7.2, 8, 9, 10, 11.3, 11.4, and 12, wherein the amino acid positions are relative to the reference sequence corresponding to residues 12 to 343 of SEQ ID NO: 2, or relative to the reference sequence corresponding to SEQ ID NO: 2.

In some embodiments, the amino acid sequence of the engineered RNA ligases comprises at least a substitution or substitution set of an RNA ligase variant forth in Tables 3, 4, 5, 6, 7.1, 7.2, 8, 9, 10, 11.3, 11.4, and 12, wherein the amino acid positions are relative to the reference sequence corresponding to residues 12 to 343 of SEQ ID NO: 2, or relative to the reference sequence corresponding to SEQ ID NO: 2.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of SEQ ID NO: 2, 4, 106, 218, 286, 396, 436, 520, 552, or 582, or to a reference sequence corresponding to SEQ ID NO: 2, 4, 106, 218, 286, 396, 436, 520, 552, or 582.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of an even-numbered SEQ ID NO of SEQ ID NOs: 4-220, 224-252, and 270-958, or to a reference sequence corresponding to an even-numbered SEQ ID NO of SEQ ID NOs: 4-220, 224-252, and 270-958.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of SEQ ID NO: 4, 106, 218, 286, 396, 436, 520, 552, or 582, or to a reference sequence corresponding to SEQ ID NO: 4, 106, 218, 286, 396, 436, 520, 552, or 582, wherein the amino acid sequence comprises one or more substitutions relative to the reference sequence corresponding to residues 12 to 343 of SEQ ID NO: 4, 106, 218, 286, 396, 436, 520, 552, or 582, or relative to the reference sequence corresponding to SEQ ID NO: 4, 106, 218, 286, 396, 436, 520, 552, or 582.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of an even-numbered SEQ ID NO of SEQ ID NOs: 4-220, 224-252, and 270-958, or to a reference sequence corresponding to an even-numbered SEQ ID NO of SEQ ID NOs: 4-220, 224-252, and 270-958, wherein the amino acid sequence comprises one or more substitutions relative to the reference sequence corresponding to residues 12 to 343 of SEQ ID NO: 4, 106, 218, 286, 396, 436, 520, 552, or 582, or relative to the reference sequence corresponding to SEQ ID NO: 4, 106, 218, 286, 396, 436, 520, 552, or 582.

In some embodiments, the amino acid sequence of the engineered RNA ligase comprises at least a substitution at amino acid position 11, 12, 14, 16, 17, 23, 25, 26, 30, 32, 33, 38, 40, 52, 57, 60, 68, 69, 71, 73, 75, 79, 82, 83, 92, 93, 95, 96, 98, 100, 101, 102, 103, 111, 114, 117, 118, 119, 120, 135, 136, 141, 142, 145, 147, 156, 167, 168, 170, 171, 174, 177, 179, 183, 184, 185, 188, 189, 190, 191, 192, 193, 196, 200, 202, 205, 206, 207, 209, 221, 229, 254, 255, 259, 270, 271, 287, 288, 289, 292, 295, 296, 300, 307, 320, 327, 332, 333, 334, 335, 336, 337, 339, 342, or 343, or combinations thereof, wherein the amino acid positions are relative to the reference sequence corresponding to residues 12 to 343 of SEQ ID NO: 4, 106, 218, 286, 396, 436, 520, 552, or 582, or relative to the reference sequence corresponding to SEQ ID NO: 4, 106, 218, 286, 396, 436, 520, 552, or 582.

In some embodiments, the amino acid sequence of the engineered RNA ligase comprises at least a substitution at amino acid position 33, 38, 71, 73, 75, 96, 98, 101, 114, 117, 136, 156, 179, 184, 191, 196, 221, 289, 292, 296, 320, 335, 336, or 339, or combinations thereof, wherein the amino acid positions are relative to the reference sequence corresponding to residues 12 to 343 of SEQ ID NO: 4, 106, 218, 286, 396, 436, 520, 552, or 582, or relative to the reference sequence corresponding to SEQ ID NO: 4, 106, 218, 286, 396, 436, 520, 552, or 582.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of SEQ ID NO: 4, or to a reference sequence corresponding to SEQ ID NO: 4, wherein the amino acid sequence comprises one or more substitutions relative to the reference sequence corresponding to residues 12 to 343 of SEQ ID NO: 4, or relative to the reference sequence corresponding to SEQ ID NO: 4.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of an even-numbered SEQ ID NO. of SEQ ID NOs: 4-104, or to a reference sequence corresponding to an even-numbered SEQ ID NO. of SEQ ID NOs: 4-104, wherein the amino acid sequence comprises one or more substitutions relative to the reference sequence corresponding to residues 12 to 343 of SEQ ID NO: 4, or relative to the reference sequence corresponding to SEQ ID NO: 4.

In some embodiments, the amino acid sequence of the engineered RNA ligase comprises at least a substitution set at amino acid position(s) 33/101/221/320/335/336/339, 71/92/171/184/202, 40/92/171/202/327, 196/300/335/339, 33/184/221/300/320, 335/336/339, 184/196/221/336, 33/184/320/335/336, 207/209/221/300/320/336/339, 101/196/221/300/335, 207/209/300/320, 40/170/171/202/327, 33/101/184/196/209/336/339, 40/71/92/171/184/327, 207/335/336/339, 33/101/196/300/320/335/336/339, 57/171/179/184/202/327, 57/92/202/327, 33/101/207/221/300/335/336/339, 33/221/336, 196/221/300/335/336/339, 33/184/221/336/339, 33/184/196/221/300, 101/196/207/209/221/300/336/339, 101/184/335/336/339, 33/184/196/320/336/339, 33/184/196/300/320/335/336/339, 184/320/335/336/339, 184/196/207/209/300/335/336/339, 184/196/320/335/336/339, 33/196/209/221/339, 184/320/335/336/339, 101/209/335/336/339, 101/196/209/339, 40/71/82/170/171/179/202/327, 320/335/336/339, 101/184/300/335/336/339, 33/101/196/209/335/336/339, 33/196/300/320, 221/335, 33/184/196/207/221, 221/335/336/339, 196/209/300/335/336/339, 184/196/300/320, 57/171/202/327, 40/71/171/184, 184/196/221, 33/196/207/335/336, 32/57/71/202/327/342, 33/196/221/336, 101/221/300/320/335/336/339, 33/101/207/221, 101/184/196/209/221, 33/207/209/300/335/336/339, or 33/83/184/196/300/336, wherein the amino acid positions are relative to the reference sequence corresponding to residues 12 to 343 of SEQ ID NO: 4, or relative to the reference sequence corresponding to SEQ ID NO: 4.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of SEQ ID NO: 106, or to a reference sequence corresponding to SEQ ID NO: 106, wherein the amino acid sequence comprises one or more substitutions relative to the reference sequence corresponding to residues 12 to 343 of SEQ ID NO: 106, or relative to the reference sequence corresponding to SEQ ID NO: 106.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of an even-numbered SEQ ID NO of SEQ ID NOs: 218-220 and 224-252, or to a reference sequence corresponding to an even-numbered SEQ ID NO of SEQ ID NOs: 218-220 and 224-252, wherein the amino acid sequence comprises one or more substitutions relative to the reference sequence corresponding to residues 12 to 343 of SEQ ID NO: 106, or relative to the reference sequence corresponding to SEQ ID NO: 106.

In some embodiments, the amino acid sequence of the engineered RNA ligase comprises at least a substitution or substitution set at amino acid position(s) 38/71/114/184/191/196, 141/170/171/174, 114, 57/60/170/171/174/192/196/320, 60/92/170/171, 141/174/196, 57/170/171/174, 141/184, 57/60/141/170/171/174/192, 60/170/174, 141/170/171, 69/71/114/184, 38/40, 40/71/98/184/259, 32/60/141/170/171/174/196/320, 32/170/207/320, or 196, wherein the amino acid positions are relative to the reference sequence corresponding to residues 12 to 343 of SEQ ID NO: 106, or relative to the reference sequence corresponding to SEQ ID NO: 106.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of SEQ ID NO: 218, or to a reference sequence corresponding to SEQ ID NO: 218, wherein the amino acid sequence comprises one or more substitutions relative to the reference sequence corresponding to residues 12 to 343 of SEQ ID NO: 218, or relative to the reference sequence corresponding to SEQ ID NO: 218.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of an even-numbered SEQ ID NO of SEQ ID NOs: 270-312, or to a reference sequence corresponding to an even-numbered SEQ ID NO of SEQ ID NOs: 270-312, wherein the amino acid sequence comprises one or more substitutions relative to the reference sequence corresponding to residues 12 to 343 of SEQ ID NO: 218, or relative to the reference sequence corresponding to SEQ ID NO: 218.

In some embodiments, the amino acid sequence of the engineered RNA ligase comprises at least a substitution or substitution set at amino acid position(s) 102, 17, 190, 16, 68, 23, 270, 117/229, or 117, wherein the amino acid positions are relative to the reference sequence corresponding to residues 12 to 343 of SEQ ID NO: 218, or relative to the reference sequence corresponding to SEQ ID NO: 218.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of SEQ ID NO: 286, or to a reference sequence corresponding to SEQ ID NO: 286, wherein the amino acid sequence comprises one or more substitutions relative to the reference sequence corresponding to residues 12 to 343 of SEQ ID NO: 286, or relative to the reference sequence corresponding to SEQ ID NO: 286.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of an even-numbered SEQ ID NO of SEQ ID NOs: 314-426, or to a reference sequence corresponding to an even-numbered SEQ ID NO of SEQ ID NOs: 314-426, wherein the amino acid sequence comprises one or more substitutions relative to the reference sequence corresponding to residues 12 to 343 of SEQ ID NO: 286, or relative to the reference sequence corresponding to SEQ ID NO: 286.

In some embodiments, the amino acid sequence of the engineered RNA ligase comprises at least a substitution or substitution set at amino acid position(s) 1296, 114/117, 26, 30, 117/118, 117/119, 14, 183, 117, 185, 307, 12, 93/135, 135, 92, 288, 117/120, 79, 100, 287, 292, 25, 75, 327, or 111, wherein the amino acid positions are relative to the reference sequence corresponding to residues 12 to 343 of SEQ ID NO: 286, or relative to the reference sequence corresponding to SEQ ID NO: 286.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of SEQ ID NO: 396, or to a reference sequence corresponding to SEQ ID NO: 396, wherein the amino acid sequence comprises one or more substitutions relative to the reference sequence corresponding to residues 12 to 343 of SEQ ID NO: 396, or relative to the reference sequence corresponding to SEQ ID NO: 396.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of an even-numbered SEQ ID NO of SEQ ID NOs: 428-488, or to a reference sequence corresponding to an even-numbered SEQ ID NO of SEQ ID NOs: 428-488, wherein the amino acid sequence comprises one or more substitutions relative to the reference sequence corresponding to residues 12 to 343 of SEQ ID NO: 396, or relative to the reference sequence corresponding to SEQ ID NO: 396.

In some embodiments, the amino acid sequence of the engineered RNA ligase comprises at least a substitution or substitution set at amino acid position(s) 102/119, 75/102, 75/102/117, 75/119, 75/136/296, 75/327, 75/117/119, 119, 119/296, 14/102/119, 14/102/296, 14/75, 14/75/119, 14/75/119/327, 14/75/117/119, 14/75/117/119/296, 14/119, 14/296, 14/30/270, 14/25, 14/117, 14/117/119, 14/117/119/296, 14/117/296/327, 26/75/327, 25/75, 25/30/102, 117, 117/119, or 117/119/296, wherein the amino acid positions are relative to the reference sequence corresponding to residues 12 to 343 of SEQ ID NO: 396, or relative to the reference sequence corresponding to SEQ ID NO: 396.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of SEQ ID NO: 436, or to a reference sequence corresponding to SEQ ID NO: 436, wherein the amino acid sequence comprises one or more substitutions relative to the reference sequence corresponding to residues 12 to 343 of SEQ ID NO: 436, or relative to the reference sequence corresponding to SEQ ID NO: 436.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of an even-numbered SEQ ID NO of SEQ ID NOs: 490-534, or to a reference sequence corresponding to an even-numbered SEQ ID NO of SEQ ID NOs: 490-534, wherein the amino acid sequence comprises one or more substitutions relative to the reference sequence corresponding to residues 12 to 343 of SEQ ID NO: 436, or relative to the reference sequence corresponding to SEQ ID NO: 436.

In some embodiments, the amino acid sequence of the engineered RNA ligase comprises at least a substitution at amino acid position 332, 156, 177, 333, 96, 11, 189, 327, 25, 73, 98, 335, 95, 188, or 52, or combinations thereof, wherein the amino acid positions are relative to the reference sequence corresponding to residues 12 to 343 of SEQ ID NO: 436, or relative to the reference sequence corresponding to SEQ ID NO: 436.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of SEQ ID NO: 520, or to a reference sequence corresponding to SEQ ID NO: 520, wherein the amino acid sequence comprises one or more substitutions relative to the reference sequence corresponding to residues 12 to 343 of SEQ ID NO: 520, or relative to the reference sequence corresponding to SEQ ID NO: 520.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of an even-numbered SEQ ID NO of SEQ ID NOs: 536-554, or to a reference sequence corresponding to an even-numbered SEQ ID NO of SEQ ID NOs: 536-554, wherein the amino acid sequence comprises one or more substitutions relative to the reference sequence corresponding to residues 12 to 343 of SEQ ID NO: 520, or relative to the reference sequence corresponding to SEQ ID NO: 520.

In some embodiments, the amino acid sequence of the engineered RNA ligase comprises at least a substitution set at amino acid position(s) 96/98/156/189/335, 73/96/98/189/335, 73/177/189, 73/98/177/189/335, 25/73/95/96/98/189/327, 25/98/189, 73/95/96/156/177, 73/335, 73/96/98/156/335, or 73/177/189/333, wherein the amino acid positions are relative to the reference sequence corresponding to residues 12 to 343 of SEQ ID NO: 520, or relative to the reference sequence corresponding to SEQ ID NO: 520.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of SEQ ID NO: 552, or to a reference sequence corresponding to SEQ ID NO: 552, wherein the amino acid sequence comprises one or more substitutions relative to the reference sequence corresponding to residues 12 to 343 of SEQ ID NO: 552, or relative to the reference sequence corresponding to SEQ ID NO: 552.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of an even-numbered SEQ ID NO of SEQ ID NOs: 556-584, or to a reference sequence corresponding to an even-numbered SEQ ID NO of SEQ ID NOs: 556-584, wherein the amino acid sequence comprises one or more substitutions relative to the reference sequence corresponding to residues 12 to 343 of SEQ ID NO: 552, or relative to the reference sequence corresponding to SEQ ID NO: 552.

In some embodiments, the amino acid sequence of the engineered RNA ligase comprises at least a substitution at amino acid position 292, 147, 255, 295, 75, 300, 333, 200, 289, 95, or 168, or combinations thereof, wherein the amino acid positions are relative to the reference sequence corresponding to residues 12 to 343 of SEQ ID NO: 552, or relative to the reference sequence corresponding to SEQ ID NO: 552.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of SEQ ID NO: 582, or to a reference sequence corresponding to SEQ ID NO: 582, wherein the amino acid sequence comprises one or more substitutions relative to the reference sequence corresponding to residues 12 to 343 of SEQ ID NO: 582, or relative to the reference sequence corresponding to SEQ ID NO: 582.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of an even-numbered SEQ ID NO of SEQ ID NOs: 586-958, or to a reference sequence corresponding to an even-numbered SEQ ID NO of SEQ ID NOs: 586-958, wherein the amino acid sequence comprises one or more substitutions relative to the reference sequence corresponding to residues 12 to 343 of SEQ ID NO: 582, or relative to the reference sequence corresponding to SEQ ID NO: 582.

In some embodiments, the amino acid sequence of the engineered RNA ligase comprises at least a substitution or substitution set at amino acid position(s) 95/156/337, 189/200/271, 200/271/289/337, 200/271/289, 189/200, 156/200/289, 156/168/189/271/300, 156/189/200/289, 156/168/289, 95/156/271/289, 156/333, 95/200/289/337, 168/200/289, 189/200/289, 333/337, 103/156/271, 95/156/200/300/333, 156/271/289/333, 95/156/271, 95/189/289, 95/156/168/189/271/289/300, 189/271/289, 95/168/271/300, 156/189/289/337, 200/271, 156/189/333, 189/289, 156/189/200/300/333/337, 156/189/200/271/289/337, 156/200/271/289/333/337, 168/271/300, 289/337, 156/168/337, 156/168/189/289, 156/271/289, 189/300, 156/289/300/337, 168/189/289, 95/156/168/300, 156/200/300/337, 289/333/337, 95/189/200/300/337, 95/156/189/200/271/333, 200/271/289/300, 289/333, 189/271/300, 95/300, 95/189/333/337, 95/189/271/300, 95/189/200/289, 168, 156/200/271/289/337, 95/168/289, 156/289, 95/156/189/271/289, 156/168/189/200/289/333, 156/300/333/337, 95/156/189/200/271/337, 271, 156/271, 156/271/300/333, 95/168/200, 168/271, 95/200/333, 189/200/271/289/333, 95/168/189/333/337, 189, 189/333/337, 156/189/271/289/333, 289, 95/156/200/271/289, 289/300/333, 95/189/289/337, 95/156/168/189/289, 271/289, 333, 168/337, 156/189/271/289, 95/156/189/337, 156/189/271/300, 156/168/289/333, 168/189/289/333, 95/333, 333/343, 156/189/289, 95/289/300, 95/156/189/200/300/333/337, 189/200/271/289/337, 95/156/168/189/289/337, 95/289, 156/289/333/337, 156/289/337, 337, 168/289, 95/156/300, 95/156/289, 95/189/271/333/337, 95/168/300, 95/156/168/271/333/337, 95/189/289/333/337, 156/168/200/289/334, 95/156/168/189/271/289, 156/189/200/289/337, 200/289, 200/289/333/337, 95/168/189/200/289, 168/200, 156/168/271/289, 189/206/289, 156/200/289/333/335, 156/200/289/333, 189/200/289/333, 189/289/335, 189/254/289/333, 167/189/206/289/333/335, 156/200/333, 156/200/289/335, 156/189/289/335, 335, 156/167/189/289, 156/189/200/289/335, 200/289/333/335, 156/189/200/289/333, 254/333/335, 189/254/333/335, 189/335, 156/189/254/289, 156/289/333, 289/335, 189/200/254/289/333, 156/189/289/333, 25/156/189/289, 156/189/206/289/333/335, 289/333/335, 156/254/289/333, 156/289/333/335, 254/289, 200/289/333, 156/189/200, 156/254/333/335, 156, 254/289/335, 156/206/289, 156/289/335, 206/289/333/335, 156/189/254/289/333, 156/254/289, 200/289/335, 156/189/206/289, 156/189/200/206/254/289/333, 25/254/333, 189/289/333/335, 156/189/200/289/333/335, 254/289/333, or 156/200/333/335, wherein the amino acid positions are relative to the reference sequence corresponding to residues 12 to 343 of SEQ ID NO: 582, or relative to the reference sequence corresponding to SEQ ID NO: 582.

In some embodiments, the amino acid sequence of the engineered RNA ligase comprises at least one substitution set forth in Tables 3, 4, 5, 6, 7.1, 7.2, 8, 9, 10, 11.3, 11.4, and 12, wherein the amino acid positions are relative to the reference sequence corresponding to residues 12 to 343 of SEQ ID NO: 2, 4, 106, 218, 286, 396, 436, 520, 552, or 582, or relative to the reference sequence corresponding to SEQ ID NO: 2, 4, 106, 218, 286, 396, 436, 520, 552, or 582.

In some embodiments, the amino acid sequence of the engineered RNA ligase comprises at least a substitution or substitution set as set forth in Tables 3, 4, 5, 6, 7.1, 7.2, 8, 9, 10, 11.3, 11.4, and 12, wherein the amino acid positions are relative to the reference sequence corresponding to residues 12 to 343 of SEQ ID NO: 2, 4, 106, 218, 286, 396, 436, 520, 552, or 582, or relative to the reference sequence corresponding to SEQ ID NO: 2, 4, 106, 218, 286, 396, 436, 520, 552, or 582.

In some embodiments, the engineered RNA ligase comprises an amino acid sequence having least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence comprising a substitution or substitution set of an RNA ligase variant set forth in Tables 3, 4, 5, 6, 7.1, 7.2, 8, 9, 10, 11.3, 11.4, and 12, wherein the amino acid positions are relative to the reference sequence corresponding to residues 12 to 343 of SEQ ID NO: 2, 4, 106, 218, 286, 396, 436, 520, 552, or 582, or relative to the reference sequence corresponding to SEQ ID NO: 4, 106, 218, 286, 396, 436, 520, 552, or 582.

In some embodiments, the engineered RNA ligase comprises an amino acid sequence comprising residues 12 to 343 of an even numbered SEQ ID NO. of SEQ ID NOs: 4-220, 224-252, and 270-958, or an amino acid sequence comprising an even numbered SEQ ID NO. of SEQ ID NOs: 4-220, 224-252, and 270-958.

In some embodiments, the engineered RNA ligase comprises an amino acid sequence comprising residues 12 to 343 of SEQ ID NO: 4, 106, 218, 286, 396, 436, 520, 552, or 582, or an amino acid sequence comprising SEQ ID NO: 4, 106, 218, 286, 396, 436, 520, 552, or 582.

In some embodiments, the engineered RNA ligase has RNA ligase activity and at least one improved property as compared to a reference RNA ligase. In some embodiments, the improved property of the engineered RNA ligase is selected from i) increased activity, ii) increased product yield, iii) increased product yield on polynucleotides with phosphorothioate internucleoside linkages, iv) increased product yield on oligonucleotides with 2′-modifications, and v) increased expression, or any combination of i), ii), iii), iv), and v), compared to a reference RNA ligase.

In some embodiments, the improved property of the engineered RNA ligase is in comparison to the reference RNA ligase having the sequence corresponding to residues 12 to 343 of SEQ ID NO: 2, 4, 106, 218, 286, 396, 436, 520, 552, or 582, or the sequence corresponding to SEQ ID NO: 2, 4, 106, 218, 286, 396, 436, 520, 552, or 582. In some embodiments, the reference RNA ligase has the sequence corresponding to residues 12 to 343 of SEQ ID NO: 2, or the sequence corresponding to SEQ ID NO: 2.

In some embodiments, the engineered RNA ligase comprises a fusion protein. In some embodiments, the engineered RNA ligase is fused to a tag or affinity polypeptide.

In some further embodiments, the engineered RNA ligase is purified. In some embodiments, the engineered RNA ligase is provided in solution, or is immobilized on a substrate, such as surfaces of solid substrates or membranes or particles.

In another aspect, the present disclosure provides a recombinant polynucleotide encoding any of the engineered RNA ligases disclosed herein.

In some embodiments, the recombinant polynucleotide comprises a polynucleotide sequence having at least 70%, 75%, 75%, 80%, 81%, 82%, 83%, 84%, 85%, 90%, 91%, 92%, 93%, 94%, 95%, 96%, 97%, 98%, 99%, or more sequence identity to a reference polynucleotide sequence corresponding to nucleotide residues 34 to 1029 of SEQ ID NO: 3, 105, 217, 285, 395, 435, 519, 551, or 581, or to a reference polynucleotide sequence corresponding to SEQ ID NO: 3, 105, 217, 285, 395, 435, 519, 551, or 581, wherein the recombinant polynucleotide encodes an RNA ligase.

In some embodiments, the recombinant polynucleotide comprises a polynucleotide sequence having at least 70%, 75%, 80%, 81%, 82%, 83%, 84%, 85%, 90%, 91%, 92%, 93%, 94%, 95%, 96%, 97%, 98%, 99%, or more sequence identity to a reference polynucleotide sequence corresponding to nucleotide residues 34 to 1029 of an odd numbered SEQ ID NO. of SEQ ID NOs: 3-219, 223-251, and 269-957, or to a reference polynucleotide sequence corresponding an odd numbered SEQ ID NO. of SEQ ID NOs: 3-219, 223-957, and 269-553, wherein the recombinant polynucleotide encodes an RNA ligase.

In some embodiments, the polynucleotide sequence of the recombinant polynucleotide encoding an engineered RNA ligase is codon optimized for expression in an organism or cell type thereof, for example a bacterial cell, fungal cell, or mammalian cell.

In some embodiments, the recombinant polynucleotide comprises a polynucleotide sequence comprising nucleotide residues 34 to 1029 of SEQ ID NO. 3, 105, 217, 285, 395, 435, 519, 551, or 581, or a polynucleotide sequence comprising SEQ ID NOs: 3, 105, 217, 285, 395, 435, 519, 551, or 581.

In some embodiments, the recombinant polynucleotide comprises a polynucleotide sequence comprising nucleotide residues 34 to 1029 of an odd numbered SEQ ID NO. of SEQ ID NOs: 3-219, 223-251, and 269-957, or a polynucleotide sequence comprising an odd numbered SEQ ID NO. of SEQ ID NOs: 3-219, 223-251, and 269-957.

In a further aspect, the present disclosure provides an expression vector comprising a recombinant polynucleotide provided herein encoding an engineered RNA ligase. In some embodiments, the recombinant polynucleotide of the expression vector is operably linked to a control sequence. In some embodiments, the control sequence comprises at least a promoter, particularly a heterologous promoter.

In another aspect, the present disclosure also provides a host cell comprising an expression vector provided herein. In some embodiments, the host cell is a prokaryotic cell or a eukaryotic cell. In some embodiments, the host cell is a bacterial cell, fungal cell, or mammalian cell. In some embodiments, the host cell is a bacterial cell, such as E. coli. or B. subtilis.

In a further aspect, the present disclosure provides a method of producing an engineered RNA ligase polypeptide, the method comprising culturing a host cell described herein under suitable culture conditions such that at least one engineered RNA ligase is produced. In some embodiments, the method further comprises recovering or isolating the engineered RNA ligase from the culture and/or host cells. In some embodiments, the method further comprises purifying the engineered RNA ligase.

In another aspect, the present disclosure provides a composition comprising an engineered RNA ligase disclosed herein. In some embodiments, the composition comprises at least a buffer. In some embodiments, the composition further comprises a nucleotide substrate (e.g., ATP or dATP) and one or more of polynucleotide ligase substrates.

In a further aspect, the present disclosure provides a method of ligating polynucleotide substrates, particularly double stranded polynucleotide substrates. In some embodiments, a method of ligating at least a first polynucleotide strand and a second polynucleotide strand, comprises contacting a first polynucleotide strand and a second polynucleotide strand with an engineered RNA ligase described herein in presence of a nucleotide substrate under conditions suitable for ligation of the first polynucleotide strand to the second polynucleotide strand, wherein the first polynucleotide strand comprises a ligatable 5′-end and the second polynucleotide strand comprises a 3′-end ligatable to the 5-end of the first polynucleotide strand. In some embodiments, the first polynucleotide strand and/or second polynucleotide strand comprises RNA or a mixture of RNA and DNA.

In some embodiments, the method further comprises a third polynucleotide strand, wherein the first polynucleotide strand and second polynucleotide strand hybridize adjacent to one another on the third polynucleotide to position the 5′-end of the first polynucleotide strand adjacent to the 3′-end of the second polynucleotide strand to form a ligatable nick. In some embodiments, the third polynucleotide strand is continuous with the first polynucleotide strand or second polynucleotide strand. In some embodiments, the third polynucleotide strand is continuous with the first polynucleotide strand and second polynucleotide strand to form a single continuous polynucleotide ligase substrate. In some embodiments, 3′-end region of the second polynucleotide strand that hybridizes to the third polynucleotide strand is at least 4, 6, 8 or more base pairs in length. In some embodiments, the 5-end region of the first polynucleotide strand that hybridizes to the third polynucleotide strand is at least 2, 3, 4, 6, 8 or more base pairs in length.

In some embodiments of the method, the third polynucleotide strand comprises a splint or bridging polynucleotide, wherein the 5′-terminal sequence of the first polynucleotide strand and the 3′-terminal sequence of the second polynucleotide strand hybridize adjacent to one another on the splint or bridging polynucleotide to position the 5′-end of the first polynucleotide strand adjacent to the 3′-end of the second polynucleotide strand.

In some embodiments, the first polynucleotide strand is hybridized to the third polynucleotide strand to form a first double stranded fragment, and the second polynucleotide strand is hybridized to a fourth polynucleotide strand to form a second double stranded fragment, where the first and second double stranded fragments have complementary ends that can base pair to form a substrate for the engineered RNA ligase. In some embodiments, the first double stranded fragment and the second double stranded fragment have complementary overhangs or complementary single stranded ends that can base pair and form double stranded nick that serves as a substrate for the engineered RNA ligase.

In some embodiments of the method, at least 2, 3, 4, 5, or 6 or more double stranded fragments, where each double stranded fragment has a complementary end that can base pair with at least one other double stranded fragment with a complementary end is used as substrates for the RNA ligase.

In some embodiments, the polynucleotide substrate comprises one or more modified nucleotides. In some embodiments, the modification comprises a modified sugar residue, a modified nucleobase, and/or modified phosphate group. In some embodiments, the modified sugar residue is modified at the 2-position of the sugar moiety. In some embodiments, the modified 2-position is a 2′-halo or 2′-O-alkyl, preferably a lower alkyl, e.g., methyl or ethyl. In some embodiments, the modified nucleotide is a modified phosphate group, for example a phosphorothioate group. In some embodiments, the modified phosphate group is at the 5′-end of a polynucleotide substrate. In some embodiments, the modified phosphate group is at an internucleoside linkage of the polynucleotide substrate. In some embodiments, the modified nucleotide has a modified nucleobase. In some embodiments, the polynucleotide substrate includes an inverted nucleotide (e.g., 5′-5′ or 3′-3′ inverted linkage). In some embodiments, the modified nucleotide is modified with a cell targeting moiety, such as a GalNac or lipid (e.g., cholesterol). In some embodiments, the modified nucleotide is modified with a linker moiety. In some embodiments, modification with the cell-targeting or linker moiety is on the nucleobase or the sugar residue. In some embodiments, the cell-targeting or linker moiety is attached to the 5′ or 3′ end of the polynucleotide substrate.

In some embodiments, the engineered RNA ligase is used in a method to synthesize RNA or DNA/RNA polynucleotides by ligation of shorter RNA or DNA/RNA oligonucleotides. In some embodiments, the engineered RNA ligase is used to ligate the 3′ OH of RNA to the 5′ phosphate of DNA or RNA. In some embodiments, the engineered RNA ligase is used to ligate the 3′ OH of RNA to the 5′ phosphate of DNA in a double-stranded-format NGS RNA library construction. In some embodiments, the engineered RNA ligase is used in a method of preparing RNA rings. In some embodiments, the engineered RNA ligase is used to repair nicks in dsRNA or dsRNA/DNA. In some embodiments, the engineered RNA ligase is used to synthesize modified RNA oligonucleotides.

In a further aspect, the present disclosure also provides a kit comprising at least one engineered RNA ligase disclosed herein. In some embodiments, the kits further comprises one or more of a buffer, nucleotide substrate (e.g., ATP or dATP), and/or one or more polynucleotide ligase substrates.

### DETAILED DESCRIPTION

The present disclosure provides engineered RNA ligase polypeptides and compositions thereof, as well as polynucleotides encoding the engineered RNA ligase polypeptides. The disclosure also provides methods of using of the engineered RNA ligase polypeptides and compositions thereof for molecular biological, diagnostic, and other purposes. In some embodiments, the engineered RNA ligase polypeptides display, among others, increased activity, increased stability, increased thermal stability, and/or increased activity on polynucleotides containing one or more modified nucleotides or nucleotide analogs.

### Abbreviations and Definitions

In reference to the present invention, the technical and scientific terms used in the descriptions herein will have the meanings commonly understood by one of ordinary skill in the art, unless specifically defined otherwise. Accordingly, the following terms are intended to have the following meanings and are to be more fully described by reference to the application as a whole.

As used herein, the singular forms “a”, “an” and “the” include plural referents unless the context clearly indicates otherwise. Thus, for example, reference to “a polypeptide” includes more than one polypeptide.

Similarly, “comprise,” “comprises,” “comprising” “include,” “includes,” and “including” are interchangeable and not intended to be limiting. Thus, as used herein, the term “comprising” and its cognates are used in their inclusive sense (i.e., equivalent to the term “including” and its corresponding cognates).

It is to be further understood that where descriptions of various embodiments use the term “comprising,” those skilled in the art would understand that in some specific instances, an embodiment can be alternatively described using language “consisting essentially of” or “consisting of.”

Moreover, numeric ranges are inclusive of the numbers defining the range. Thus, every numerical range disclosed herein is intended to encompass every narrower numerical range that falls within such broader numerical range, as if such narrower numerical ranges were all expressly written herein. It is also intended that every maximum (or minimum) numerical limitation disclosed herein includes every lower (or higher) numerical limitation, as if such lower (or higher) numerical limitations were expressly written herein.

As used herein, the term “about” means an acceptable error for a particular value. In some instances, “about” means within 0.05%, 0.5%, 1.0%, or 2.0%, of a given value range. In some instances, “about” means within 1, 2, 3, or 4 standard deviations of a given value.

Furthermore, the headings provided herein are not limitations of the various aspects or embodiments of the invention which can be had by reference to the application as a whole. Accordingly, the terms defined immediately below are more fully defined by reference to the application as a whole.

“EC” number refers to the Enzyme Nomenclature of the Nomenclature Committee of the International Union of Biochemistry and Molecular Biology (NC-IUBMB). The IUBMB biochemical classification is a numerical classification system for enzymes based on the chemical reactions they catalyze.

“ATCC” refers to the American Type Culture Collection whose biorepository collection includes genes and strains.

“NCBI” refers to National Center for Biological Information and the sequence databases provided therein.

“RNA ligase” refers to enzymes that covalently joins the 5′-phosphoryl termini of RNA or DNA to the 3′-hydroxyl termini of RNA or DNA. Two families of RNA ligases are known to occur in nature. RNA ligase 1 catalyzes the covalent joining of single stranded 5′-phosphoryl termini of RNA or DNA to single stranded 3-hydroxyl termini of RNA or DNA. RNA ligase 2 also catalyzes the covalent joining of a 3′-hydroxyl terminus of RNA to a 5′-phosphorylated RNA or DNA but shows preference for double stranded substrates. RNA ligases include those enzymes classified as EC 6.5.1.3.

Although some DNA ligases are capable of acting on either DNA or RNA as the 3′-hydroxyl strand substrate, an RNA ligase 2 acting at a duplex nick preferentially acts on 3′-hydroxyl strand of RNA but is agnostic to whether 5-phosphoryl strand is DNA or RNA. Moreover, in some embodiments, the strand with the 3′-hydroxyl may include deoxyribonucleotides if sufficient number of ribonucleotides are present at the 3′-hydroxyl terminus. For example, the RNA specificity of T4 RNA ligase 2 is affected by the 3′-hydroxyl strand, and specifically by the two terminal ribonucleotides of the 3-′hydroxyl side of the nick (see, e.g., Nandakumar et al., Mol. Cell., 2004, 16:211-221). It is to be understood that the ligation reaction is not limited to naturally occurring RNA and DNA substrates also includes polynucleotide substrates that contain modified nucleotides and/or nucleotide analogs.

“Protein,” “polypeptide,” and “peptide” are used interchangeably to denote a polymer of at least two amino acids covalently linked by an amide bond, regardless of length or post-translational modification (e.g., glycosylation or phosphorylation).

“Amino acids” are referred to herein by either their commonly known three-letter symbols or by the one-letter symbols recommended by IUPAC-IUB Biochemical Nomenclature Commission. Nucleotides, likewise, may be referred to by their commonly accepted single letter codes. The abbreviations used for the genetically encoded amino acids are conventional and are as follows: alanine (Ala or A), arginine (Arg or R), asparagine (Asn or N), aspartate (Asp or D), cysteine (Cys or C), glutamate (Glu or E), glycine (Gly or G), glutamine (Gln or Q), histidine (His or H), isoleucine (Ile or I), leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y), and valine (Val or V). When the three-letter abbreviations are used, unless specifically preceded by an “L” or a “D” or clear from the context in which the abbreviation is used, the amino acid may be in either the L- or D-configuration about α-carbon (C_{α}). For example, whereas “Ala” designates alanine without specifying the configuration about the α-carbon, “D-Ala” and “L-Ala” designate D-alanine and L-alanine, respectively. When the one-letter abbreviations are used, upper case letters designate amino acids in the L-configuration about the α-carbon and lower case letters designate amino acids in the D-configuration about the α-carbon. For example, “A” designates L-alanine and “a” designates D-alanine. When polypeptide sequences are presented as a string of one-letter or three-letter abbreviations (or mixtures thereof), the sequences are presented in the amino (N) to carboxy (C) direction in accordance with common convention.

“Fusion protein,” and “chimeric protein” and “chimera” refer to hybrid proteins created through the joining of two or more polynucleotides that originally encode separate proteins. In some embodiments, fusion proteins are created by recombinant technology (e.g., molecular biology techniques known in the art).

“Polynucleotide,” “nucleic acid,” or “oligonucleotide” is used herein to denote a polymer comprising at least two nucleotides where the nucleotides are either deoxyribonucleotides or ribonucleotides or mixtures of deoxyribonucleotides and ribonucleotides. In some embodiments, the abbreviations used for genetically encoding nucleosides are conventional and are as follow: adenosine (A); guanosine (G); cytidine (C); thymidine (T); and uridine (U). Unless specifically delineated, the abbreviated nucleosides may be either ribonucleosides or 2′-deoxyribonucleosides. The nucleosides may be specified as being either ribonucleosides or 2′-deoxyribonucleosides on an individual basis or on an aggregate basis. When a polynucleotide, nucleic acid, or oligonucleotide sequences are presented as a string of one-letter abbreviations, the sequences are presented in the 5′ to 3′ direction in accordance with common convention, and the phosphates are not indicated. The term “DNA” refers to deoxyribonucleic acid. The term “RNA” refers to ribonucleic acid. The polynucleotide or nucleic acid may be single-stranded or double-stranded, or may include both single-stranded regions and double-stranded regions.

The terms “polynucleotide,” “nucleic acid” and “oligonucleotide” in some embodiments encompass polynucleotide or nucleic acid or oligonucleotide analogs, which include, among others, nucleosides linked together via other than standard phosphodiester linkages, such as non-standard linkages of phosphoramidates, phosphorothioates, amide linkages, etc.; nucleosides/nucleotides with non-standard nucleobases, including modified and/or synthetic nucleobases, for example inosine, xanthine, hypoxanthine, etc.; nucleosides with modified sugar residues, such as 2′-O-alkyl, 2′-halo, 2,3-dideoxy, 2′-halo-2′deoxy, β-D-ribo LNA, α-L-ribo-LNA (locked nucleic acids), etc.; and/or 5′-phosphate analogs, including, among others, phosphorothioate, phosphoacetate, phosphoramidate, monomethylphosphate, methylphosphonate, or phosphonocarboxylate.

“Duplex” and “ds” refer to a double-stranded nucleic acid (e.g., DNA or RNA) molecule comprised of two single-stranded polynucleotides that are complementary in their sequence (e.g., A pairs to T or U, C pairs to G), arranged in an antiparallel 5′ to 3′ orientation, and held together by hydrogen bonds between the nucleobases (e.g., adenine [A], guanine [G], cytosine [C], thymine [T], uridine [U]). In some embodiments, the duplex or double stranded nucleic acid is formed using modified nucleobases or nucleobase analogs.

“Complementary” is used herein to describe the structural relationship between nucleotide bases that are capable of forming base pairs with one another. For example, a purine nucleotide base present on a polynucleotide that is complementary to a pyrimidine nucleotide base on a polynucleotide may base pair by forming hydrogen bonds with one another. Complementary nucleotide bases can base pair via Watson/Crick base pairing or in any other manner than forms stable duplexes or other nucleic acid structures.

“Watson/Crick Base-Pairing” refers to a pattern of specific pairs of nucleobases and analogs that bind together through sequence-specific hydrogen-bonds, e.g., A pairs with T or U, and G pairs with C.

“Annealing” or “Hybridization” refers to the base-pairing interactions of one nucleobase polymer (e.g., poly- and oligonucleotides) with another that results in the formation of a double-stranded structure, a triplex structure or a quaternary structure. Annealing or hybridization can occur via Watson-Crick base-pairing interactions, but may be mediated by other hydrogen-bonding interactions, such as Hoogsteen base pairing. In some embodiments, the nucleobase polymer that anneals or hybridizes to another is a single nucleobase polymer while in other embodiments, the nucleobase polymers are separate nucleobase polymers.

“Engineered,” “recombinant,” “non-naturally occurring,” and “variant,” when used with reference to a cell, a polynucleotide or a polypeptide refer to a material or a material corresponding to the natural or native form of the material that has been modified in a manner that would not otherwise exist in nature or is identical thereto but produced or derived from synthetic materials and/or by manipulation using recombinant techniques.

“Wild-type” and “naturally-occurring” refer to the form found in nature. For example, a wild-type polypeptide or polynucleotide sequence is a sequence present in an organism that can be isolated from a source in nature and which has not been intentionally modified by human manipulation.

“Coding sequence” refers to that part of a nucleic acid (e.g., a gene) that encodes an amino acid sequence of a protein.

“Percent (%) sequence identity” refers to comparisons among polynucleotides and polypeptides, and are determined by comparing two optimally aligned sequences over a comparison window, wherein the portion of the polynucleotide or polypeptide sequence in the comparison window may comprise additions or deletions (i.e., gaps) as compared to the reference sequence for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted, e.g., by the local homology algorithm of Smith and Waterman (Smith and Waterman, Adv. Appl. Math., 1981, 2:482), by the homology alignment algorithm of Needleman and Wunsch (Needleman and Wunsch, J. Mol. Biol., 1970, 48:443), by the search for similarity method of Pearson and Lipman (Pearson and Lipman, Proc. Natl. Acad. Sci. USA, 1988, 85:2444), by computerized implementations of these algorithms (e.g., GAP, BESTFIT, FASTA, and TFASTA in the GCG Wisconsin Software Package), or by visual inspection, as known in the art. Examples of algorithms that are suitable for determining percent sequence identity and sequence similarity include, but are not limited to the BLAST and BLAST 2.0 algorithms (see, e.g., Altschul et al., J. Mol. Biol., 1990, 215:403-410; and Altschul et al., Nucleic Acids Res., 1977, 3389-3402). Software for performing BLAST analyses is publicly available through the National Center for Biotechnology Information website. This algorithm involves first identifying high scoring sequence pairs (HSPs) by identifying short words of length “W” in the query sequence, which either match or satisfy some positive-valued threshold score “T,” when aligned with a word of the same length in a database sequence. T is referred to as the neighborhood word score threshold (see Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters “M” (reward score for a pair of matching residues; always >0) and “N” (penalty score for mismatching residues; always <0). For amino acid sequences, a scoring matrix is used to calculate the cumulative score. Extension of the word hits in each direction are halted when: the cumulative alignment score falls off by the quantity “X” from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see, e.g., Henikoff and Henikoff, Proc. Natl. Acad. Sci. USA, 1989, 89:10915). Exemplary determination of sequence alignment and % sequence identity can employ the BESTFIT or GAP programs in the GCG Wisconsin Software package (Accelrys, Madison WI), using default parameters provided.

“Reference sequence” refers to a defined sequence used as a basis for a sequence comparison. A reference sequence may be a subset of a larger sequence, for example, a segment of a full-length gene or polypeptide sequence. Generally, a reference sequence is at least 20 nucleotide or amino acid residues in length, at least 25 residues in length, at least 50 residues in length, at least 100 residues in length or the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptide are typically performed by comparing sequences of the two polynucleotides or polypeptides over a “comparison window” to identify and compare local regions of sequence similarity. In some embodiments, a “reference sequence” can be based on a primary amino acid sequence, where the reference sequence is a sequence that can have one or more changes in the primary sequence. For instance, the phrase “a reference sequence corresponding to SEQ ID NO: 14, having a leucine at the residue corresponding to X14” (or “a reference sequence corresponding to SEQ ID NO: 14, having a lysine at the residue corresponding to position 14”) refers to a reference sequence in which the corresponding residue at position X14 in SEQ ID NO: 14 (e.g., an alanine), has been changed to lysine.

“Comparison window” refers to a conceptual segment of contiguous nucleotide positions or amino acids residues wherein a sequence may be compared to a reference sequence. In some embodiments, the comparison window is at least 15 to 20 contiguous nucleotides or amino acids and wherein the portion of the sequence in the comparison window may comprise additions or deletions (i.e., gaps) of 20 percent or less as compared to the reference sequence (which does not comprise additions or deletions) for optimal alignment of the two sequences. In some embodiments, the comparison window can be longer than 15-20 contiguous residues, and includes, optionally 30, 40, 50, 100, or longer windows.

“Corresponding to”, “reference to,” and “relative to” when used in the context of the numbering of a given amino acid or polynucleotide sequence refer to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence, such as that of an engineered RNA ligase, can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned. In some embodiments, the sequence is tagged (e.g., with a histidine tag).

“Mutation” refers to the alteration of a nucleic acid sequence. In some embodiments, mutations result in changes to the encoded polypeptide sequence (i.e., as compared to the original sequence without the mutation). In some embodiments, the mutation comprises a substitution, such that a different amino acid is produced. In some alternative embodiments, the mutation comprises an addition, such that an amino acid is added (e.g., insertion) to the original polypeptide sequence. In some further embodiments, the mutation comprises a deletion, such that an amino acid is deleted from the original polypeptide sequence. Any number of mutations may be present in a given sequence.

“Amino acid difference” and “residue difference” refer to a difference in the amino acid residue at a position of a polypeptide sequence relative to the amino acid residue at a corresponding position in a reference sequence. The positions of amino acid differences generally are referred to herein as “Xn,” where n refers to the corresponding position in the reference sequence upon which the residue difference is based. For example, a “residue difference at position X17 as compared to SEQ ID NO: 2” (or a “residue difference at position 17 as compared to SEQ ID NO: 2”) refers to a difference of the amino acid residue at the polypeptide position corresponding to position 17 of SEQ ID NO: 2. Thus, if the reference polypeptide of SEQ ID NO: 2 has a serine at position 17, then a “residue difference at position X17 as compared to SEQ ID NO: 2” refers to an amino acid substitution of any residue other than serine at the position of the polypeptide corresponding to position 17 of SEQ ID NO: 2. In some instances herein, the specific amino acid residue difference at a position is indicated as “XnY” where “Xn” specified the corresponding residue and position of the reference polypeptide (as described above), and “Y” is the single letter identifier of the amino acid found in the engineered polypeptide (i.e., the different residue than in the reference polypeptide). In some instances (e.g., in the Tables in the Examples), the present disclosure also provides specific amino acid differences denoted by the conventional notation “AnB”, where A is the single letter identifier of the residue in the reference sequence, “n” is the number of the residue position in the reference sequence, and B is the single letter identifier of the residue substitution in the sequence of the engineered polypeptide. In some instances, an amino acid residue difference or substitution may be a deletion and may be denoted by a “−”. In some embodiments, the amino acid difference, e.g., a substitution, is denoted by the abbreviation “nB,” without the identifier for the residue in the reference sequence. In some embodiments, the phrase “an amino acid residue nB” denotes the presence of the amino residue in the engineered polypeptide, which may or may not be a substitution in context of a reference polypeptide or amino acid sequence.

In some instances, a polypeptide of the present disclosure can include one or more amino acid residue differences relative to a reference sequence, which is indicated by a list of the specified positions where residue differences are present relative to the reference sequence. In some embodiments, where more than one amino acid can be used in a specific residue position of a polypeptide, the various amino acid residues that can be used are separated by a “/” (e.g., T33A/T33K, T33A/K, or 33A/K).

“Amino acid substitution set” and “substitution set” refers to a group of amino acid substitutions within a polypeptide sequence. In some embodiments, substitution sets comprise 2, 3, 4, 5, 6, 7, 8, 9, 10, 11, 12, 13, 14, 15, or more amino acid substitutions. In some embodiments, a substitution set refers to the set of amino acid substitutions that is present in any of the variant RNA ligase polypeptides listed in any of the Tables in the Examples. In these substitution sets, the individual substitutions are separated by a semicolon (“;”; e.g., N141T; A184P) or slash (“/”; e.g., N141T/A184P or 141T/184P).

“Conservative amino acid substitution” refers to a substitution of a residue with a different residue having a similar side chain, and thus typically involves substitution of the amino acid in the polypeptide with amino acids within the same or similar defined class of amino acids. By way of example and not limitation, an amino acid with an aliphatic side chain may be substituted with another aliphatic amino acid (e.g., alanine, valine, leucine, and isoleucine); an amino acid with hydroxyl side chain is substituted with another amino acid with a hydroxyl side chain (e.g., serine and threonine); an amino acids having aromatic side chains is substituted with another amino acid having an aromatic side chain (e.g., phenylalanine, tyrosine, tryptophan, and histidine); an amino acid with a basic side chain is substituted with another amino acid with a basis side chain (e.g., lysine and arginine); an amino acid with an acidic side chain is substituted with another amino acid with an acidic side chain (e.g., aspartic acid or glutamic acid); and a hydrophobic or hydrophilic amino acid is replaced with another hydrophobic or hydrophilic amino acid, respectively.

“Non-conservative substitution” refers to substitution of an amino acid in the polypeptide with an amino acid with significantly differing side chain properties. Non-conservative substitutions may use amino acids between, rather than within, the defined groups and affect: (a) the structure of the peptide backbone in the area of the substitution (e.g., proline for glycine); (b) the charge or hydrophobicity; and/or (c) the bulk of the side chain. By way of example and not limitation, exemplary non-conservative substitutions include an acidic amino acid substituted with a basic or aliphatic amino acid; an aromatic amino acid substituted with a small amino acid; and a hydrophilic amino acid substituted with a hydrophobic amino acid.

“Deletion” refers to modification to the polypeptide by removal of one or more amino acids from the reference polypeptide. Deletions can comprise removal of 1 or more amino acids, 2 or more amino acids, 5 or more amino acids, 10 or more amino acids, 15 or more amino acids, or 20 or more amino acids, up to 10% of the total number of amino acids, or up to 20% of the total number of amino acids making up the reference enzyme while retaining enzymatic activity and/or retaining the improved properties of an engineered RNA ligase. Deletions can be directed to the internal portions and/or terminal portions of the polypeptide. In various embodiments, the deletion can comprise a continuous segment or can be discontinuous. Deletions are indicated by “−”, and may be present in substitution sets.

“Insertion” refers to modification to the polypeptide by addition of one or more amino acids from the reference polypeptide. Insertions can be in the internal portions of the polypeptide, or to the carboxy or amino terminus. Insertions as used herein include fusion proteins as is known in the art. The insertion can be a contiguous segment of amino acids or separated by one or more of the amino acids in the naturally occurring polypeptide.

“Functional fragment” and “biologically active fragment” are used interchangeably herein, to refer to a polypeptide that has an amino-terminal and/or carboxy-terminal deletion(s) and/or internal deletions, but where the remaining amino acid sequence is identical to the corresponding positions in the sequence to which it is being compared (e.g., a full length engineered RNA ligase of the present invention) and that retains substantially all of the activity of the full-length polypeptide.

“Isolated polypeptide” refers to a polypeptide which is substantially separated from other contaminants that naturally accompany it (e.g., protein, lipids, and polynucleotides). The term embraces polypeptides which have been removed or purified from their naturally-occurring environment or expression system (e.g., host cell or in vitro synthesis). The recombinant RNA ligase polypeptides may be present within a cell, present in the cellular medium, or prepared in various forms, such as lysates or isolated preparations. As such, in some embodiments, the recombinant RNA ligase polypeptides provided herein are isolated polypeptides.

“Substantially pure polypeptide” refers to a composition in which the polypeptide species is the predominant species present (i.e., on a molar or weight basis it is more abundant than any other individual macromolecular species in the composition), and is generally a substantially purified composition when the object species comprises at least about 50 percent of the macromolecular species present by mole or % weight. Generally, a substantially pure RNA ligase composition will comprise about 60% or more, about 70% or more, about 80% or more, about 90% or more, about 95% or more, and about 98% or more of all macromolecular species by mole or % weight present in the composition. In some embodiments, the object species is purified to essential homogeneity (i.e., contaminant species cannot be detected in the composition by conventional detection methods) wherein the composition consists essentially of a single macromolecular species. Solvent species, small molecules (<500 Daltons), and elemental ion species are not considered macromolecular species. In some embodiments, the isolated recombinant RNA ligase polypeptides are substantially pure polypeptide compositions.

“Improved enzyme property” refers to an engineered RNA ligase polypeptide that exhibits an improvement in any enzyme property as compared to a reference RNA ligase polypeptide, such as a wild-type RNA ligase polypeptide or another engineered RNA ligase polypeptide. Improved properties include but are not limited to such properties as increased protein expression, increased thermoactivity, increased thermostability, increased stability, increased enzymatic activity, increased substrate specificity and/or affinity, increased substrate range, increased specific activity, increased resistance to substrate and/or end-product inhibition, increased chemical stability, improved solvent stability, increased solubility, and increased inhibitor resistance or tolerance.

“Increased enzymatic activity” and “enhanced catalytic activity” refer to an improved property of the engineered RNA ligase polypeptides, which can be represented by an increase in specific activity (e.g., product produced/time/weight protein) and/or an increase in percent conversion of the substrate to the product (e.g., percent conversion of starting amount of substrate to product in a specified time period using a specified amount of RNA ligase) as compared to the reference RNA ligase enzyme (e.g., wild-type RNA ligase and/or another engineered RNA ligase). Exemplary methods to determine enzyme activity are provided in the Examples. Any property relating to enzyme activity may be affected, including the classical enzyme properties of Kₘ, Vₘₐₓ or k_{cat}, changes of which can lead to increased enzymatic activity. Improvements in enzyme activity can be from about 1.1 fold the enzymatic activity of the corresponding wild-type enzyme, to about 1.5 fold, 2-fold, 5-fold, 10-fold, 20-fold, 25-fold, 50-fold, 75-fold, 100-fold, 150-fold, 200-fold or more enzymatic activity than the naturally occurring RNA ligase or another engineered RNA ligase from which the RNA ligase polypeptides were derived.

“Hybridization stringency” relates to hybridization conditions, such as washing conditions, in the hybridization of nucleic acids. Generally, hybridization reactions are performed under conditions of lower stringency, followed by washes of varying but higher stringency (see, e.g., Sambrook et al., Molecular Cloning: A Laboratory Manual, Cold Spring Harbor Laboratory Press, New York, 2001; Ausubel et al., Current Protocols in Molecular Biology, John Wiley & Sons, 2003). The term “moderately stringent hybridization” refers to conditions that permit target-DNA to bind a complementary nucleic acid that has about 60% identity, preferably about 75% identity, about 85% identity to the target DNA, with greater than about 90% identity to target-polynucleotide. Exemplary moderately stringent conditions are conditions equivalent to hybridization in 50% formamide, 5× Denhart's solution, 5×SSPE, 0.2% SDS at 42° C., followed by washing in 0.2×SSPE, 0.2% SDS, at 42° C. “High stringency hybridization” refers generally to conditions that are about 10° C. or less from the thermal melting temperature Tₘ as determined under the solution condition for a defined polynucleotide sequence. In some embodiments, a high stringency condition refers to conditions that permit hybridization of only those nucleic acid sequences that form stable hybrids in 0.018M NaCl at 65° C. (i.e., if a hybrid is not stable in 0.018M NaCl at 65° C., it will not be stable under high stringency conditions, as contemplated herein). High stringency conditions can be provided, for example, by hybridization in conditions equivalent to 50% formamide, 5× Denhart's solution, 5×SSPE, 0.2% SDS at 42° C., followed by washing in 0.1×SSPE, and 0.1% SDS at 65° C. Another high stringency condition comprises hybridizing in conditions equivalent to hybridizing in 5×SSC containing 0.1% (w:v) SDS at 65° C. and washing in 0.1×SSC containing 0.1% SDS at 65° C. Other high stringency hybridization conditions, as well as moderately stringent conditions, are described in the references cited above.

“Codon optimized” refers to changes in the codons of the polynucleotide encoding a protein to those preferentially used in a particular organism such that the encoded protein is more efficiently expressed in that organism. Although the genetic code is degenerate, in that most amino acids are represented by several codons, called “synonyms” or “synonymous” codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism's genome. In some embodiments, the polynucleotides encoding the RNA ligase enzymes are codon optimized for optimal production from the host organism selected for expression.

“Control sequence” refers herein to include all components that are necessary or advantageous for the expression of a polynucleotide and/or polypeptide of the present disclosure. Each control sequence may be native or foreign to the nucleic acid sequence encoding the polypeptide. Such control sequences include, but are not limited to, leaders, polyadenylation sequences, propeptide sequences, promoter sequences, signal peptide sequences, initiation sequences, and transcription terminators. At a minimum, the control sequences include a promoter, and transcriptional and translational stop signals. In some embodiments, the control sequences are provided with linkers for the purpose of introducing specific restriction sites facilitating ligation of the control sequences with the coding region of the nucleic acid sequence encoding a polypeptide.

“Operably linked” or “operatively linked” refers to a configuration in which a control sequence is appropriately placed (i.e., in a functional relationship) at a position relative to a polynucleotide of interest such that the control sequence directs or regulates the expression of the polynucleotide encoding a polypeptide of interest.

“Promoter” or “promoter sequence” refers to a nucleic acid sequence that is recognized by a host cell for expression of a polynucleotide of interest, such as a coding sequence. The promoter sequence contains transcriptional control sequences that mediate the expression of a polynucleotide of interest. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell.

“Suitable reaction conditions” or “suitable conditions” refers to those conditions in the enzymatic conversion reaction solution (e.g., ranges of enzyme loading, substrate loading, temperature, pH, buffers, co-solvents, etc.) under which an RNA ligase polypeptide of the present disclosure is capable of converting a polynucleotide(s) substrate to the desired ligated product polynucleotide. Exemplary “suitable reaction conditions” are provided herein (see, the Examples).

“Product” in the context of an enzymatic conversion process refers to the compound or molecule resulting from the action of the RNA ligase polypeptide on the substrate.

“Culturing” refers to the growing of a population of microbial cells under suitable conditions using any suitable medium (e.g., liquid, gel, or solid).

“Vector” is a recombinant construct for introducing a polynucleotide of interest into a cell. In some embodiments, the vector is an expression vector that is operably linked to a suitable control sequence capable of effecting the expression in a suitable host of the polynucleotide or a polypeptide encoded in the polynucleotide. In some embodiments, an “expression vector” has a promoter sequence operably linked to the polynucleotide (e.g., transgene) to drive expression in a host cell, and in some embodiments, also comprises a transcription terminator sequence.

“Expression” includes any step involved in the production of the polypeptide including, but not limited to, transcription, post-transcriptional modification, translation, and post-translational modification. In some embodiments, the term also encompasses secretion of the polypeptide from a cell.

“Produces” refers to the production of proteins and/or other compounds by cells. It is intended that the term encompass any step involved in the production of polypeptides including, but not limited to, transcription, post-transcriptional modification, translation, and post-translational modification. In some embodiments, the term also encompasses secretion of the polypeptide from a cell.

“Heterologous” or “recombinant” refers to the relationship between two or more nucleic acid or polypeptide sequences (e.g., a promoter sequence, signal peptide, terminator sequence, etc.) that are derived from different sources and are not associated in nature.

“Host cell” and “host strain” refer to suitable hosts for expression vectors comprising a polynucleotide provided herein (e.g., a polynucleotide sequences encoding at least one RNA ligase variant). In some embodiments, the host cells are prokaryotic or eukaryotic cells that have been transformed or transfected with vectors constructed using recombinant DNA techniques as known in the art.

### Engineered RNA Ligase Polypeptides

In one aspect, the present disclosure provides RNA ligases, including engineered RNA ligase polypeptide variants. In some embodiments, the RNA ligase and engineered RNA ligase polypeptide variants are useful for ligating polynucleotide substrates, such as for preparing polynucleotides from short oligonucleotides, and for diagnostic and other purposes. The engineered RNA ligase variants can be used in solution, as well as in immobilized embodiments. In some embodiments, the engineered RNA ligase can be prepared and used as non-fusion polypeptides or as fusion polypeptides.

In some embodiments, the engineered RNA ligase, or functional fragment thereof, comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of an even numbered SEQ ID NO. of SEQ ID NOs: 2-220, 224-252, and 270-958, or to a reference sequence corresponding to an even numbered SEQ ID NO. of SEQ ID NOs: 2-220, 224-252, and 270-958, wherein the amino acid sequence comprises one or more substitutions relative to the reference sequence corresponding to residues 12 to 343 of SEQ ID NO: 2, 4, 106, 218, 286, 396, 436, 520, 552, or 582, or relative to the reference sequence corresponding to SEQ ID NO: 2, 4, 106, 218, 286, 396, 436, 520, 552, or 582.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of SEQ ID NO: 2, 4, 106, 218, 286, 396, 436, 520, 552, or 582, or to a reference sequence corresponding to SEQ ID NO: 2, 4, 106, 218, 286, 396, 436, 520, 552, or 582, wherein the amino acid sequence comprises one or more substitutions relative to the reference sequence corresponding to residues 12 to 343 of SEQ ID NO: 2, 4, 106, 218, 286, 396, 436, 520, 552, or 582, or relative to the reference sequence corresponding to SEQ ID NO: 2, 4, 106, 218, 286, 396, 436, 520, 552, or 582.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 343 of SEQ ID NO: 2, or to the reference sequence corresponding to SEQ ID NO: 2, wherein the amino acid sequence comprises one or more substitutions relative to the reference sequence corresponding to residues 12 to 343 of SEQ ID NO: 2, or relative to the reference sequence corresponding to SEQ ID NO: 2.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 343 of SEQ ID NO: 4, 106, 218, 286, 396, 436, 520, 552, or 582, or to the reference sequence corresponding to SEQ ID NO: 4, 106, 218, 286, 396, 436, 520, 552, or 582, wherein the amino acid sequence comprises one or more substitutions relative to the reference sequence corresponding to residues 12 to 343 of SEQ ID NO: 2, or relative to the reference sequence corresponding to SEQ ID NO: 2.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 343 of an even-numbered SEQ ID NO. of SEQ ID NOs: 4-220, 224-252, and 270-958, or to a reference sequence corresponding to an even-numbered SEQ ID NO. of SEQ ID NOs: 4-220, 224-252, and 270-958, wherein the amino acid sequence comprises one or more substitutions relative to the reference sequence corresponding to residues 12 to 343 of SEQ ID NO: 2, or relative to the reference sequence corresponding to SEQ ID NO: 2.

In some embodiments, the amino acid sequence of the engineered RNA ligase comprises at least a substitution at amino acid position 11, 12, 14, 16, 17, 23, 25, 26, 30, 32, 33, 38, 40, 52, 57, 60, 68, 69, 71, 73, 75, 79, 82, 83, 92, 93, 95, 96, 98, 100, 101, 102, 103, 111, 114, 117, 118, 119, 120, 135, 136, 141, 142, 145, 147, 156, 167, 168, 170, 171, 174, 177, 179, 183, 184, 185, 188, 189, 190, 191, 192, 193, 196, 200, 202, 205, 206, 207, 209, 221, 229, 254, 255, 259, 270, 271, 287, 288, 289, 292, 295, 296, 300, 307, 320, 327, 332, 333, 334, 335, 336, 337, 339, 342, or 343, or combinations thereof, wherein the amino acid positions are relative to the reference sequence corresponding to residues 12 to 343 of SEQ ID NO: 2, or relative to the reference sequence corresponding to SEQ ID NO: 2.

In some embodiments, the amino acid sequence of the engineered RNA ligase comprises at least a substitution or amino acid residue 11P, 12S, 14F/R/T/W, 16R, 17P, 23M, 25A/G/K/L/W, 26F/G/H/L/P/R/S/V, 30A/C/H/R, 32R, 33A/K, 38D, 40E, 52L, 57T, 60R, 68W, 69L, 71K, 73T, 75L/Q/T, 79W, 82I, 83P, 92A/E/G/M/N/S, 93A, 95E/V, 96A/S, 98D/E/P/R/S, 100R, 101G, 102T, 103I, 111L, 114F/G/K/N/P/R/S/V, 117A/C/F/G/H/I/K/L/M/R/S/T/V/W, 118G/L/Q, 119A/E/S/T, 120P, 135Q/T, 136A, 141A/C/E/T, 142H/R, 145D, 147L, 156C/Y, 167A, 168A, 170E, 171E, 174Q, 177I/L, 179F, 183V, 184L/M/P/R, 185H/S, 188S, 189L/V, 190F, 191K, 192V, 193N, 196K, 200S/T, 202W, 205R/V, 206R, 207S/T, 209E/L, 221E, 229A, 254G, 255E/S, 259V, 270A, 271I, 287A, 288A, 289A/H/S/T, 292E/Y, 295R, 296E/L/R/V/W, 300E/K, 307A, 320D/K, 327I/Q/R/W, 332R, 333A/E/S/T, 334S, 335E/H/K/L, 336S, 337G, 339K/R, 342I/T, 343P, 339K/R, or 342I/T, or combinations thereof, wherein the amino acid positions are relative to the reference sequence corresponding to residues 12 to 343 of SEQ ID NO: 2, or relative to the reference sequence corresponding to SEQ ID NO: 2.

In some embodiments, the amino acid sequence of the engineered RNA ligase comprises at least a substitution or amino acid residue G11P, M12S, K14F/R/T/W, Y16R, S17P, Y23M, S25A/G/K/L/W, K26F/G/H/L/P/R/S/V, K30A/C/H/R, Y32R, T33A/K, T38D, V40E, F52L, E57T, N60R, G68W, P69L, L71K, A73T, D75L/Q/T, Y79W, V82I, L83P, T92A/E/G/M/N/S, V93A, K95E/V, F96A/S, Y98D/E/P/R/S, A100R, R101G, A102T, V103I, F111L, G114F/K/N/P/R/S/V, Q117A/C/F/G/H/I/K/L/M/R/S/T/V/W, K118G/L/Q, G119A/E/S/T, V120P, N135Q/T, T136A, N141A/C/E/T, T142H/R, T145D, Y147L, F156C/Y, G167A, T168A, D170E, S171E, M174Q, N177I/L, L179F, L183V, A184L/M/P/R, A185H/S, A188S, T189L/V, A190F, S191K, E192V, D193N, E196K, C200S/T, F202W, N205R/V, V206R, I207S/T, D209E/L, F221E, T229A, V254G, P255E/S, I259V, Y270A, V271I, T287A, P288A, K289A/H/S/T, G292E/Y, M295R, G296E/L/R/V/W, Q300E/K, S307A, N320D/K, V327I/Q/R/W, D332R, V333A/E/S/T, L334S, R335E/H/K/L, P336S, A337G, I339K/R, V342I/T, or S343P, or combinations thereof, wherein the amino acid positions are relative to the reference sequence corresponding to residues 12 to 343 of SEQ ID NO: 2, or relative to the reference sequence corresponding to SEQ ID NO: 2.

In some embodiments, the amino acid sequence of the engineered RNA ligase comprises at least a substitution at amino acid position 3, 38, 71, 73, 75, 96, 98, 101, 114, 117, 136, 156, 179, 184, 191, 196, 221, 289, 292, 296, 320, 335, 336, or 339, or combinations thereof, wherein the amino acid positions are relative to the reference sequence corresponding to residues 12 to 343 of SEQ ID NO: 2, or relative to the reference sequence corresponding to SEQ ID NO: 2.

In some embodiments, the amino acid sequence of the engineered RNA ligase comprises at least a substitution or amino acid residue 33A/K, 38D, 71K, 73T, 75L/Q/T, 96A/S, 98D/E/P/R/S, 101G, 114F/K/N/P/R/S/V, 117A/C/F/G/H/I/K/L/M/R/S/T/V/W, 136A, 156C/Y, 179F, 184L/M/P/R, 191K, 196K, 221E, 289A/H/S/T, 292E/Y, 296E/L/R/V/W, 320D/K, 335E/H/K/L, 336S, or 339K/R, or combinations thereof, wherein the amino acid positions are relative to the reference sequence corresponding to residues 12 to 343 of SEQ ID NO: 2, or relative to the reference sequence corresponding to SEQ ID NO: 2.

In some embodiments, the amino acid sequence of the engineered RNA ligase comprises at least a substitution or amino acid residue 33A, 38D, 71K, 73T, 75T, 96S, 98S, 101G, 114N, 117W, 136A, 156C, 179F, 184M, 191K, 196K, 221E, 289H, 292E, 296W, 320D, 335E/K, 336S, or 339R, or combinations thereof, wherein the amino acid positions are relative to the reference sequence corresponding to residues 12 to 343 of SEQ ID NO: 2, or relative to the reference sequence corresponding to SEQ ID NO: 2.

In some embodiments, the amino acid sequence of the engineered RNA ligase comprises at least a substitution or amino acid residue T33A, T38D, L71K, A73T, D75T, F96S, Y98S, R101G, G114N, Q117W, T136A, F156C, L179F, A184M, S191K, E196K, F221E, K289H, G292E, G296W, N320D, R335E/K, P336S, I339R, or combinations thereof, wherein the amino acid positions are relative to the reference sequence corresponding to residues 12 to 343 of SEQ ID NO: 2, or relative to the reference sequence corresponding to SEQ ID NO: 2.

In some embodiments, the amino acid sequence of the engineered RNA ligase comprises at least a substitution at amino acid position 179, wherein the amino acid positions are relative to the reference sequence corresponding to residues 12 to 343 of SEQ ID NO: 2, or relative to the reference sequence corresponding to SEQ ID NO: 2. In some embodiments, the amino acid sequence of the engineered RNA ligase comprises at least a substitution or amino acid residue 179F, wherein the amino acid positions are relative to the reference sequence corresponding to residues 12 to 343 of SEQ ID NO: 2, or relative to the reference sequence corresponding to SEQ ID NO: 2. In some embodiments, the amino acid sequence of the engineered RNA ligase comprises at least a substitution L179F, wherein the amino acid positions are relative to the reference sequence corresponding to residues 12 to 343 of SEQ ID NO: 2, or relative to the reference sequence corresponding to SEQ ID NO: 2.

In some embodiments, the amino acid sequence of the engineered RNA ligase comprises at least a substitution at amino acid position 33, 101, 221, 320, 335, 336, or 339, or combinations thereof, wherein the amino acid positions are relative to the reference sequence corresponding to residues 12 to 343 of SEQ ID NO: 2, or relative to the reference sequence corresponding to SEQ ID NO: 2. In some embodiments, the amino acid sequence of the engineered RNA ligase comprises at least a substitution 33A, 101G, 221E, 320D, 335E, 336S, or 339R, or combinations thereof, wherein the amino acid positions are relative to the reference sequence corresponding to residues 12 to 343 of SEQ ID NO: 2, or relative to the reference sequence corresponding to SEQ ID NO: 2. In some embodiments, the amino acid sequence of the engineered RNA ligase comprises at least a substitution T33A, R101G, F221E, N320D, R335E, P336S, 1339R, or combinations thereof, wherein the amino acid positions are relative to the reference sequence corresponding to residues 12 to 343 of SEQ ID NO: 2, or relative to the reference sequence corresponding to SEQ ID NO: 2. In some embodiments, the amino acid sequence of the engineered RNA ligase comprises at least a substitution set at amino acid positions 33/101/221/320/335/336/339, wherein the amino acid positions are relative to the reference sequence corresponding to residues 12 to 343 of SEQ ID NO: 2, or relative to the reference sequence corresponding to SEQ ID NO: 2. In some embodiments, the amino acid sequence of the engineered RNA ligase comprises at least a substitution set or amino acid residues 33A/101G/221E/320D/335E/336S/339R, wherein the amino acid positions are relative to the reference sequence corresponding to residues 12 to 343 of SEQ ID NO: 2, or relative to the reference sequence corresponding to SEQ ID NO: 2. In some embodiments, the amino acid sequence of the engineered RNA ligase comprises at least a substitution set or amino acid residues T33A/R101G/F221E/N320D/R335E/P336S/1339R, wherein the amino acid positions are relative to the reference sequence corresponding to residues 12 to 343 of SEQ ID NO: 2, or relative to the reference sequence corresponding to SEQ ID NO: 2.

In some embodiments, the amino acid sequence of the engineered RNA ligase comprises at least a substitution at amino acid position 38, 71, 114, 184, 191, or 196, or combinations thereof, wherein the amino acid positions are relative to the reference sequence corresponding to residues 12 to 343 of SEQ ID NO: 2, or relative to the reference sequence corresponding to SEQ ID NO: 2. In some embodiments, the amino acid sequence of the engineered RNA ligase comprises at least a substitution or amino acid residue 38D, 71K, 114N, 184M, 191K, or 196K, or combinations thereof, wherein the amino acid positions are relative to the reference sequence corresponding to residues 12 to 343 of SEQ ID NO: 2, or relative to the reference sequence corresponding to SEQ ID NO: 2. In some embodiments, the amino acid sequence of the engineered RNA ligase comprises at least a substitution or amino acid residue T38D, L71K, G114N, A184M, S191K, or E196K, or combinations thereof, wherein the amino acid positions are relative to the reference sequence corresponding to residues 12 to 343 of SEQ ID NO: 2, or relative to the reference sequence corresponding to SEQ ID NO: 2. In some embodiments, the amino acid sequence of the engineered RNA ligase comprises at least a substitution set at amino acid positions 38/71/114/184/191/196, wherein the amino acid positions are relative to the reference sequence corresponding to residues 12 to 343 of SEQ ID NO: 2, or relative to the reference sequence corresponding to SEQ ID NO: 2. In some embodiments, the amino acid sequence of the engineered RNA ligase comprises at least a substitution set or amino acid residues 38D/71K/114N/184M/191K/196K, wherein the amino acid positions are relative to the reference sequence corresponding to residues 12 to 343 of SEQ ID NO: 2, or relative to the reference sequence corresponding to SEQ ID NO: 2. In some embodiments, the amino acid sequence of the engineered RNA ligase comprises at least a substitution set or amino acid residues T38D/L71K/G114N/A184M/S191K/E196K, or combinations thereof, wherein the amino acid positions are relative to the reference sequence corresponding to residues 12 to 343 of SEQ ID NO: 2, or relative to the reference sequence corresponding to SEQ ID NO: 2.

In some embodiments, the amino acid sequence of the engineered RNA ligase comprises at least a substitution at amino acid position 117, wherein the amino acid positions are relative to the reference sequence corresponding to residues 12 to 343 of SEQ ID NO: 2, or relative to the reference sequence corresponding to SEQ ID NO: 2. In some embodiments, the amino acid sequence of the engineered RNA ligase comprises at least a substitution or amino acid residue 117W, wherein the amino acid positions are relative to the reference sequence corresponding to residues 12 to 343 of SEQ ID NO: 2, or relative to the reference sequence corresponding to SEQ ID NO: 2. In some embodiments, the amino acid sequence of the engineered RNA ligase comprises at least a substitution Q117W, wherein the amino acid positions are relative to the reference sequence corresponding to residues 12 to 343 of SEQ ID NO: 2, or relative to the reference sequence corresponding to SEQ ID NO: 2.

In some embodiments, the amino acid sequence of the engineered RNA ligase comprises at least a substitution at amino acid position 292, wherein the amino acid positions are relative to the reference sequence corresponding to residues 12 to 343 of SEQ ID NO: 2, or relative to the reference sequence corresponding to SEQ ID NO: 2. In some embodiments, the amino acid sequence of the engineered RNA ligase comprises at least a substitution 292E, wherein the amino acid positions are relative to the reference sequence corresponding to residues 12 to 343 of SEQ ID NO: 2, or relative to the reference sequence corresponding to SEQ ID NO: 2. In some embodiments, the amino acid sequence of the engineered RNA ligase comprises at least a substitution G292E, wherein the amino acid positions are relative to the reference sequence corresponding to residues 12 to 343 of SEQ ID NO: 2, or relative to the reference sequence corresponding to SEQ ID NO: 2.

In some embodiments, the amino acid sequence of the engineered RNA ligase comprises at least a substitution at amino acid position 75, 136, or 296, or combinations thereof wherein the amino acid positions are relative to the reference sequence corresponding to residues 12 to 343 of SEQ ID NO: 2, or relative to the reference sequence corresponding to SEQ ID NO: 2. In some embodiments, the amino acid sequence of the engineered RNA ligase comprises at least a substitution or amino acid residue 75T, 136A, or 296W, or combinations thereof, wherein the amino acid positions are relative to the reference sequence corresponding to residues 12 to 343 of SEQ ID NO: 2, or relative to the reference sequence corresponding to SEQ ID NO: 2. In some embodiments, the amino acid sequence of the engineered RNA ligase comprises at least a substitution or amino acid residue D75T, T136A, or G296W, or combinations thereof, wherein the amino acid positions are relative to the reference sequence corresponding to residues 12 to 343 of SEQ ID NO: 2, or relative to the reference sequence corresponding to SEQ ID NO: 2. In some embodiments, the amino acid sequence of the engineered RNA ligase comprises at least a substitution set at amino acid position 75/136/296, or combinations thereof wherein the amino acid positions are relative to the reference sequence corresponding to residues 12 to 343 of SEQ ID NO: 2, or relative to the reference sequence corresponding to SEQ ID NO: 2. In some embodiments, the amino acid sequence of the engineered RNA ligase comprises at least a substitution set or amino acid residues 75T/136A/296W, or combinations thereof, wherein the amino acid positions are relative to the reference sequence corresponding to residues 12 to 343 of SEQ ID NO: 2, or relative to the reference sequence corresponding to SEQ ID NO: 2. In some embodiments, the amino acid sequence of the engineered RNA ligase comprises at least a substitution set or amino acid residues D75T/T136A/G296W, or combinations thereof, wherein the amino acid positions are relative to the reference sequence corresponding to residues 12 to 343 of SEQ ID NO: 2, or relative to the reference sequence corresponding to SEQ ID NO: 2.

In some embodiments, the amino acid sequence of the engineered RNA ligase comprises at least a substitution at amino acid position 98, wherein the amino acid positions are relative to the reference sequence corresponding to residues 12 to 343 of SEQ ID NO: 2, or relative to the reference sequence corresponding to SEQ ID NO: 2. In some embodiments, the amino acid sequence of the engineered RNA ligase comprises at least a substitution or amino acid residue 98S, wherein the amino acid positions are relative to the reference sequence corresponding to residues 12 to 343 of SEQ ID NO: 2, or relative to the reference sequence corresponding to SEQ ID NO: 2. In some embodiments, the amino acid sequence of the engineered RNA ligase comprises at least a substitution or amino acid residue Y98S, wherein the amino acid positions are relative to the reference sequence corresponding to residues 12 to 343 of SEQ ID NO: 2, or relative to the reference sequence corresponding to SEQ ID NO: 2.

In some embodiments, the amino acid sequence of the engineered RNA ligase comprises at least a substitution at amino acid position 73, 96, 98, 156, or 335, or combinations thereof wherein the amino acid positions are relative to the reference sequence corresponding to residues 12 to 343 of SEQ ID NO: 2, or relative to the reference sequence corresponding to SEQ ID NO: 2. In some embodiments, the amino acid sequence of the engineered RNA ligase comprises at least a substitution or amino acid residue 73T, 96S, 98Y, 156C, or 335K, or combinations thereof, wherein the amino acid positions are relative to the reference sequence corresponding to residues 12 to 343 of SEQ ID NO: 2, or relative to the reference sequence corresponding to SEQ ID NO: 2. In some embodiments, the amino acid sequence of the engineered RNA ligase comprises at least a substitution or amino acid residue A73T, F96S, S98Y, F156C, or E335K, or combinations thereof, wherein the amino acid positions are relative to the reference sequence corresponding to residues 12 to 343 of SEQ ID NO: 2, or relative to the reference sequence corresponding to SEQ ID NO: 2. In some embodiments, the amino acid sequence of the engineered RNA ligase comprises at least a substitution set at amino acid positions 73/96/98/156/335, or combinations thereof wherein the amino acid positions are relative to the reference sequence corresponding to residues 12 to 343 of SEQ ID NO: 2, or relative to the reference sequence corresponding to SEQ ID NO: 2. In some embodiments, the amino acid sequence of the engineered RNA ligase comprises at least a substitution set or amino acid residues 73T/96S/98Y/156C/335K, or combinations thereof, wherein the amino acid positions are relative to the reference sequence corresponding to residues 12 to 343 of SEQ ID NO: 2, or relative to the reference sequence corresponding to SEQ ID NO: 2. In some embodiments, the amino acid sequence of the engineered RNA ligase comprises at least a substitution set or amino acid residues A73T/F96S/S98Y/F156C/E335K, or combinations thereof, wherein the amino acid positions are relative to the reference sequence corresponding to residues 12 to 343 of SEQ ID NO: 2, or relative to the reference sequence corresponding to SEQ ID NO: 2.

In some embodiments, the amino acid sequence of the engineered RNA ligase comprises at least a substitution at amino acid position 289, wherein the amino acid positions are relative to the reference sequence corresponding to residues 12 to 343 of SEQ ID NO: 2, or relative to the reference sequence corresponding to SEQ ID NO: 2. In some embodiments, the amino acid sequence of the engineered RNA ligase comprises at least a substitution or amino acid residue 289H, wherein the amino acid positions are relative to the reference sequence corresponding to residues 12 to 343 of SEQ ID NO: 2, or relative to the reference sequence corresponding to SEQ ID NO: 2. In some embodiments, the amino acid sequence of the engineered RNA ligase comprises at least a substitution or amino acid residue K289H, wherein the amino acid positions are relative to the reference sequence corresponding to residues 12 to 343 of SEQ ID NO: 2, or relative to the reference sequence corresponding to SEQ ID NO: 2.

In some embodiments, the amino acid sequence of the engineered RNA ligase comprises at least a substitution at amino acid positions 14, 32, 33, 38, 40, 57, 60, 71, 75, 82, 92, 96, 98, 101, 114, 141, 142, 145, 170, 171, 174, 179, 184, 191, 193, 196, 202, 205, 207, 209, 221, 255, 320, 327, 339, or 342, or combinations thereof, wherein the amino acid positions are relative to the reference sequence corresponding to residues 12 to 343 of SEQ ID NO: 2, or relative to the reference sequence corresponding to SEQ ID NO: 2.

In some embodiments, the amino acid sequence of the engineered RNA ligase comprises at least a substitution or amino acid residue 179F, 184P, 196K, 92A, 40E, 82I, 32R, 207T, 33K, 114K, 171E, 33A, 221E, 207S, 202W, 101G, 184R, 141C, 60R, 184L, 98R, 96S, 205V, 191K, 193N, 141A, 320K, 174Q, 98D, 71K, 142R, 57T, 320D, 205R, 209L, 145D, 14R, 209E, 342I, 255E, 327Q, 170E, 141E, 98P, 339K, 38D, 142H, 75Q, 184M, 342T, or 339R, or combinations thereof, wherein the amino acid positions are relative to the reference sequence corresponding to residues 12 to 343 of SEQ ID NO: 2, or relative to the reference sequence corresponding to SEQ ID NO: 2.

In some embodiments, the amino acid sequence of the engineered RNA ligase comprises at least a substitution or amino acid residue L179F, A184P, E196K, T92A, V40E, V82I, Y32R, I207T, T33K, G114K, S171E, T33A, F221E, I207S, F202W, R101G, A184R, N141C, N60R, A184L, Y98R, F96S, N205V, S191K, D193N, N141A, N320K, M174Q, Y98D, L71K, T142R, E57T, N320D, N205R, D209L, T145D, K14R, D209E, V342I, P255E, V327Q, D170E, N141E, Y98P, 1339K, T38D, T142H, D75Q, A184M, V342T, or I339R, or combinations thereof, wherein the amino acid positions are relative to the reference sequence corresponding to residues 12 to 343 of SEQ ID NO: 2, or relative to the reference sequence corresponding to SEQ ID NO: 2.

In some embodiments, the amino acid sequence of the engineered RNA ligase comprises at least a substitution at an amino acid position set forth in Tables 3, 4, 5, 6, 7.1, 7.2, 8, 9, 10, 11.3, 11.4, and 12, wherein the amino acid positions are relative to the reference sequence corresponding to residues 12 to 343 of SEQ ID NO: 2, or relative to the reference sequence corresponding to SEQ ID NO: 2.

In some embodiments, the amino acid sequence of the engineered RNA ligase comprises at least one substitution set forth in Tables 3, 4, 5, 6, 7.1, 7.2, 8, 9, 10, 11.3, 11.4, and 12, wherein the amino acid positions are relative to the reference sequence corresponding to residues 12 to 343 of SEQ ID NO: 2, or relative to the reference sequence corresponding to SEQ ID NO: 2.

In some embodiments, the amino acid sequence of the engineered RNA ligase comprises at least a substitution or substitution set at the amino acid position(s) set forth in Tables 3, 4, 5, 6, 7.1, 7.2, 8, 9, 10, 11.3, 11.4, and 12, wherein the amino acid positions are relative to the reference sequence corresponding to SEQ ID NO: 2.

In some embodiments, the amino acid sequence of the engineered RNA ligase comprises at least a substitution or substitution set of an RNA ligase variant set forth in Tables 3, 4, 5, 6, 7.1, 7.2, 8, 9, 10, 11.3, 11.4, and 12, wherein the amino acid positions are relative to the reference sequence corresponding to residues 12 to 343 of SEQ ID NO: 2, or relative to the reference sequence corresponding to SEQ ID NO: 2.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to a substitution or substitution set of an RNA ligase variant set forth in Tables 3, 4, 5, 6, 7.1, 7.2, 8, 9, 10, 11.3, 11.4, and 12, wherein the amino acid positions are relative to the reference sequence corresponding to residues 12 to 343 of SEQ ID NO: 2, or relative to the reference sequence corresponding to SEQ ID NO: 2.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of SEQ ID NO: 2, 4, 106, 218, 286, 396, 436, 520, 552, or 582, or to a reference sequence corresponding to SEQ ID NO: 2, 4, 106, 218, 286, 396, 436, 520, 552, or 582.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of an even-numbered SEQ ID NO of SEQ ID NOs: 4-220, 224-252, and 270-958, or to a reference sequence corresponding to an even-numbered SEQ ID NO of SEQ ID NOs: 4-220, 224-252, and 270-958.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of SEQ ID NO: 4, 106, 218, 286, 396, 436, 520, 552, or 582, or to a reference sequence corresponding to SEQ ID NO: 4, 106, 218, 286, 396, 436, 520, 552, or 582, wherein the amino acid sequence comprises one or more substitutions relative to the reference sequence corresponding to residues 12 to 343 of SEQ ID NO: 4, 106, 218, 286, 396, 436, 520, 552, or 582, or relative to the reference sequence corresponding to SEQ ID NO: 4, 106, 218, 286, 396, 436, 520, 552, or 582.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of an even-numbered SEQ ID NO of SEQ ID NOs: 4-220, 224-252, and 270-958, or to a reference sequence corresponding to an even-numbered SEQ ID NO of SEQ ID NOs: 4-220, 224-252, and 270-958, wherein the amino acid sequence comprises one or more substitutions relative to the reference sequence corresponding to residues 12 to 343 of SEQ ID NO: 4, 106, 218, 286, 396, 436, 520, 552, or 582, or relative to the reference sequence corresponding to SEQ ID NO: 4, 106, 218, 286, 396, 436, 520, 552, or 582.

In some embodiments, the amino acid sequence of the engineered RNA ligase comprises at least a substitution at amino acid position 11, 12, 14, 16, 17, 23, 25, 26, 30, 32, 33, 38, 40, 52, 57, 60, 68, 69, 71, 73, 75, 79, 82, 83, 92, 93, 95, 96, 98, 100, 101, 102, 103, 111, 114, 117, 118, 119, 120, 135, 136, 141, 142, 145, 147, 156, 167, 168, 170, 171, 174, 177, 179, 183, 184, 185, 188, 189, 190, 191, 192, 193, 196, 200, 202, 205, 206, 207, 209, 221, 229, 254, 255, 259, 270, 271, 287, 288, 289, 292, 295, 296, 300, 307, 320, 327, 332, 333, 334, 335, 336, 337, 339, 342, or 343, or combinations thereof, wherein the amino acid positions are relative to the reference sequence corresponding to residues 12 to 343 of SEQ ID NO: 4, 106, 218, 286, 396, 436, 520, 552, or 582, or relative to the reference sequence corresponding to SEQ ID NO: 4, 106, 218, 286, 396, 436, 520, 552, or 582.

In some embodiments, the amino acid sequence of the engineered RNA ligase comprises at least a substitution or an amino acid residue 11P, 12S, 14F/R/T/W, 16R, 17P, 23M, 25A/G/K/L/W, 26F/G/H/L/P/R/S/V, 30A/C/H/R, 32R, 33A/K, 38D, 40E, 52L, 57T, 60R, 68W, 69L, 71K, 73T, 75L/Q/T, 79W, 82I, 83P, 92A/E/G/M/N/S, 93A, 95E/V, 96A/S, 98D/E/P/R/S/Y, 100R, 101G, 102T, 103I, 111L, 114F/G/K/N/P/R/S/V, 117A/C/F/G/H/I/K/L/M/R/S/T/V/W, 118G/L/Q, 119A/E/S/T, 120P, 135Q/T, 136A, 141A/C/E/T, 142H/R, 145D, 147L, 156C/F/Y, 167A, 168A, 170E, 171E, 174Q, 177I/L, 179F/L, 183V, 184L/M/P/R, 185H/S, 188S, 189L/V, 190F, 191K, 192V, 193N, 196K, 200S/T, 202W, 205R/V, 206R, 207S/T, 209E/L, 221E, 229A, 254G, 255E/S, 259V, 270A, 271I, 287A, 288A, 289A/H/K/S/T, 292E/Y, 295R, 296E/L/R/V/W, 300E/K, 307A, 320D/K, 327I/Q/R/W, 332R, 333A/E/S/T, 334S, 335E/H/K/L, 336S, 337G, 339K/R, 342I/T, 343P, 339K/R, or 342I/T, or combinations thereof, wherein the amino acid positions are relative to the reference sequence corresponding to residues 12 to 343 of SEQ ID NO: 4, 106, 218, 286, 396, 436, 520, 552, or 582, or relative to the reference sequence corresponding to SEQ ID NO: 4, 106, 218, 286, 396, 436, 520, 552, or 582.

In some embodiments, the amino acid sequence of the engineered RNA ligase comprises at least a substitution at amino acid position 33, 38, 71, 73, 75, 96, 98, 101, 114, 117, 136, 156, 179, 184, 191, 196, 221, 289, 292, 296, 320, 335, 336, or 339, or combinations thereof, wherein the amino acid positions are relative to the reference sequence corresponding to residues 12 to 343 of SEQ ID NO: 4, 106, 218, 286, 396, 436, 520, 552, or 582, or relative to the reference sequence corresponding to SEQ ID NO: 4, 106, 218, 286, 396, 436, 520, 552, or 582.

In some embodiments, the amino acid sequence of the engineered RNA ligase comprises at least a substitution or an amino acid residue 33A/K, 38D, 71K, 73T, 75L/Q/T, 96A/S, 98D/E/P/R/S/Y, 101G, 114F/G/K/N/P/R/S/V, 117A/C/F/G/H/I/K/L/M/R/S/T/V/W, 136A, 156C/F/Y, 179F/L, 184L/M/P/R, 191K, 196K, 221E, 289A/H/K/S/T, 292E/Y, 296E/L/R/V/W, 320D/K, 335E/H/K/L, 336S, or 339K/R, or combinations thereof, wherein the amino acid positions are relative to the reference sequence corresponding to residues 12 to 343 of SEQ ID NO: 4, 106, 218, 286, 396, 436, 520, 552, or 582, or relative to the reference sequence corresponding to SEQ ID NO: 4, 106, 218, 286, 396, 436, 520, 552, or 582.

In some embodiments, the amino acid sequence of the engineered RNA ligase comprises at least a substitution or an amino acid residue 33A, 38D, 71K, 73T, 75T, 96S, 98S/Y, 101G, 114N, 117W, 136A, 156C, 179F, 184M, 191K, 196K, 221E, 289H, 292E, 296W, 320D, 335E/K, 336S, or 339R, or combinations thereof, wherein the amino acid positions are relative to the reference sequence corresponding to residues 12 to 343 of SEQ ID NO: 4, 106, 218, 286, 396, 436, 520, 552, or 582, or relative to the reference sequence corresponding to SEQ ID NO: 4, 106, 218, 286, 396, 436, 520, 552, or 582.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of SEQ ID NO: 4, or to a reference sequence corresponding to SEQ ID NO: 4, wherein the amino acid sequence comprises one or more substitutions relative to the reference sequence corresponding to residues 12 to 343 of SEQ ID NO: 4, or relative to the reference sequence corresponding to SEQ ID NO: 4.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of an even-numbered SEQ ID NO. of SEQ ID NOs: 4-104, or to a reference sequence corresponding to an even-numbered SEQ ID NO. of SEQ ID NOs: 4-104, wherein the amino acid sequence comprises one or more substitutions relative to the reference sequence corresponding to residues 12 to 343 of SEQ ID NO: 4, or relative to the reference sequence corresponding to SEQ ID NO: 4.

In some embodiments, the amino acid sequence of the engineered RNA ligases comprises a substitution set at amino acid positions(s) 33/101/221/320/335/336/339, 71/92/171/184/202, 40/92/171/202/327, 196/300/335/339, 33/184/221/300/320, 335/336/339, 184/196/221/336, 33/184/320/335/336, 207/209/221/300/320/336/339, 101/196/221/300/335, 207/209/300/320, 40/170/171/202/327, 33/101/184/196/209/336/339, 40/71/92/171/184/327, 207/335/336/339, 33/101/196/300/320/335/336/339, 57/171/179/184/202/327, 57/92/202/327, 33/101/207/221/300/335/336/339, 33/221/336, 196/221/300/335/336/339, 33/184/221/336/339, 33/184/196/221/300, 101/196/207/209/221/300/336/339, 101/184/335/336/339, 33/184/196/320/336/339, 33/184/196/300/320/335/336/339, 184/320/335/336/339, 184/196/207/209/300/335/336/339, 184/196/320/335/336/339, 33/196/209/221/339, 184/320/335/336/339, 101/209/335/336/339, 101/196/209/339, 40/71/82/170/171/179/202/327, 320/335/336/339, 101/184/300/335/336/339, 33/101/196/209/335/336/339, 33/196/300/320, 221/335, 33/184/196/207/221, 221/335/336/339, 196/209/300/335/336/339, 184/196/300/320, 57/171/202/327, 40/71/171/184, 184/196/221, 33/196/207/335/336, 32/57/71/202/327/342, 33/196/221/336, 101/221/300/320/335/336/339, 33/101/207/221, 101/184/196/209/221, 33/207/209/300/335/336/339, or 33/83/184/196/300/336, wherein the amino acid positions are relative to the reference sequence corresponding to residues 12 to 343 of SEQ ID NO: 4, or relative to the reference sequence corresponding to SEQ ID NO: 4.

In some embodiments, the amino acid sequence of the engineered RNA ligases comprises a substitution set or amino acid residue(s) 33A/101G/221E/320D/335E/336S/339R, 71K/92A/171E/184P/202W, 40E/92A/171E/202W/327Q, 196K/300K/335E/339R, 33A/184R/221E/300K/320D, 335E/336S/339R, 184P/196K/221E/336S, 33K/184R/320D/335E/336S, 207T/209L/221E/300K/320D/336S/339R, 101G/196K/221E/300K/335E, 207T/209L/300K/320D, 40E/170E/171E/202W/327Q, 33K/101G/184R/196K/209L/336S/339R, 40E/71K/92A/171E/184P/327Q, 207T/335E/336S/339R, 33K/101G/196K/300K/320D/335E/336S/339R, 57T/171E/179L/184P/202W/327Q, 57T/92A/202W/327Q, 33K/101G/207T/221E/300K/335E/336S/339R, 33K/221E/336S, 196K/221E/300K/335E/336S/339R, 33K/184P/221E/336S/339R, 33A/184P/196K/221E/300K, 101G/196K/207T/209L/221E/300K/336S/339R, 101G/184R/335E/336S/339R, 33A/184L/196K/320D/336S/339R, 33A/184P/196K/300K/320D/335H/336S/339R, 184L/320D/335E/336S/339R, 184L/196K/207T/209L/300K/335E/336S/339R, 184R/196K/320D/335E/336S/339R, 33K/196K/209L/221E/339R, 184R/320D/335E/336S/339R, 101G/209L/335E/336S/339R, 101G/196K/209L/339R, 40E/71K/82I/170E/171E/179L/202W/327Q, 320D/335E/336S/339R, 101G/184L/300K/335E/336S/339R, 33K/101G/196K/209L/335E/336S/339R, 33K/196K/300K/320D, 221E/335E, 33K/184R/196K/207S/221E, 221E/335E/336S/339R, 196K/209L/300K/335E/336S/339R, 184P/196K/300K/320D, 57T/171E/202W/327Q, 40E/71K/171E/184P, 184L/196K/221E, 33A/196K/207S/335E/336S, 32R/57T/71K/202W/327Q/342I, 33A/196K/221E/336S, 101G/221E/300K/320D/335E/336S/339R, 33K/184P/320D/335H/336S, 33K/101G/207S/221E, 101G/184R/196K/209L/221E, 33A/207S/209L/300K/335E/336S/339R, or 33K/83P/184P/196K/300K/336S, wherein the amino acid positions are relative to the reference sequence corresponding to residues 12 to 343 of SEQ ID NO: 4, or relative to the reference sequence corresponding to SEQ ID NO: 4.

In some embodiments, the amino acid sequence of the engineered RNA ligases comprises a substitution set or amino acid residue(s) T33A/R101G/F221E/N320D/R335E/P336S/1339R, L71K/T92A/S171E/A184P/F202W, V40E/T92A/S171E/F202W/V327Q, E196K/Q300K/R335E/1339R, T33A/A184R/F221E/Q300K/N320D, R335E/P336S/I339R, A184P/E196K/F221E/P336S, T33K/A184R/N320D/R335E/P336S, I207T/D209L/F221E/Q300K/N320D/P336S/1339R, R101G/E196K/F221E/Q300K/R335E, I207T/D209L/Q300K/N320D, V40E/D170E/S171E/F202W/V327Q, T33K/R101G/A184R/E196K/D209L/P336S/1339R, V40E/L71K/T92A/S171E/A184P/V327Q, I207T/R335E/P336S/1339R, T33K/R101G/E196K/Q300K/N320D/R335E/P336S/1339R, E57T/S171E/F179L/A184P/F202W/V327Q, E57T/T92A/F202W/V327Q, T33K/R101G/I207T/F221E/Q300K/R335E/P336S/1339R, T33K/F221E/P336S, E196K/F221E/Q300K/R335E/P336S/1339R, T33K/A184P/F221E/P336S/1339R, T33A/A184P/E196K/F221E/Q300K, R101G/E196K/I207T/D209L/F221E/Q300K/P336S/1339R, R101G/A184R/R335E/P336S/1339R, T33A/A184L/E196K/N320D/P336S/1339R, T33A/A184P/E196K/Q300K/N320D/R335H/P336S/1339R, A184L/N320D/R335E/P336S/1339R, A184L/E196K/I207T/D209L/Q300K/R335E/P336S/1339R, A184R/E196K/N320D/R335E/P336S/1339R, T33K/E196K/D209L/F221E/1339R, A184R/N320D/R335E/P336S/1339R, R101G/D209L/R335E/P336S/1339R, R101G/E196K/D209L/1339R, V40E/L71K/V82I/D170E/S171E/F179L/F202W/V327Q, N320D/R335E/P336S/1339R, R101G/A184L/Q300K/R335E/P336S/1339R, T33K/R101G/E196K/D209L/R335E/P336S/1339R, T33K/E196K/Q300K/N320D, F221E/R335E, T33K/A184R/E196K/I207S/F221E, F221E/R335E/P336S/1339R, E196K/D209L/Q300K/R335E/P336S/1339R, A184P/E196K/Q300K/N320D, E57T/S171E/F202W/V327Q, V40E/L71K/S171E/A184P, A184L/E196K/F221E, T33A/E196K/1207S/R335E/P336S, Y32R/E57T/L71K/F202W/V327Q/V342I, T33A/E196K/F221E/P336S, R101G/F221E/Q300K/N320D/R335E/P336S/1339R, T33K/A184P/N320D/R335H/P336S, T33K/R101G/I207S/F221E, R101G/A184R/E196K/D209L/F221E, T33A/I207S/D209L/Q300K/R335E/P336S/1339R, or T33K/L83P/A184P/E196K/Q300K/P336S, wherein the amino acid positions are relative to the reference sequence corresponding to residues 12 to 343 of SEQ ID NO: 4, or relative to the reference sequence corresponding to SEQ ID NO: 4.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of SEQ ID NO: 106, or to a reference sequence corresponding to SEQ ID NO: 106, wherein the amino acid sequence comprises one or more substitutions relative to the reference sequence corresponding to residues 12 to 343 of SEQ ID NO: 106, or relative to the reference sequence corresponding to SEQ ID NO: 106.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of an even-numbered SEQ ID NO of SEQ ID NOs: 218-220 and 224-252, or to a reference sequence corresponding to an even-numbered SEQ ID NO of SEQ ID NOs: 218-220 and 224-252, wherein the amino acid sequence comprises one or more substitutions relative to the reference sequence corresponding to residues 12 to 343 of SEQ ID NO: 106, or relative to the reference sequence corresponding to SEQ ID NO: 106.

In some embodiments, the amino acid sequence of the engineered RNA ligases comprises a substitution or substitution set at amino acid positions(s) 38/71/114/184/191/196, 141/170/171/174, 114, 57/60/170/171/174/192/196/320, 60/92/170/171, 141/174/196, 57/170/171/174, 141/184, 57/60/141/170/171/174/192, 60/170/174, 141/170/171, 69/71/114/184, 38/40, 40/71/98/184/259, 32/60/141/170/171/174/196/320, 32/170/207/320, or 196, wherein the amino acid positions are relative to the reference sequence corresponding to residues 12 to 343 of SEQ ID NO: 106, or relative to the reference sequence corresponding to SEQ ID NO: 106.

In some embodiments, the amino acid sequence of the engineered RNA ligases comprises a substitution or substitution set or amino acid residue(s) 38D/71K/114N/184M/191K/196K, 141A/170E/171E/174Q, 114R, 57T/60R/170E/171E/174Q/192V/196K/320K, 60R/92A/170E/171E, 141A/174Q/196K, 57T/170E/171E/174Q, 141T/184P, 57T/60R/141A/170E/171E/174Q/192V, 60R/170E/174Q, 141C/170E/171E, 69L/71K/114N/184M, 38D/40E, 40E/71K/98R/184M/259V, 32R/60R/141A/170E/171E/174Q/196K/320K, 32R/170E/207T/320K, or 196K, wherein the amino acid positions are relative to the reference sequence corresponding to residues 12 to 343 of SEQ ID NO: 106, or relative to the reference sequence corresponding to SEQ ID NO: 106.

In some embodiments, the amino acid sequence of the engineered RNA ligases comprises a substitution or substitution set or amino acid residue(s) T38D/L71K/G114N/A184M/S191K/E196K, N141A/D170E/S171E/M174Q, G114R, E57T/N60R/D170E/S171E/M174Q/E192V/E196K/D320K, N60R/T92A/D170E/S171E, N141A/M174Q/E196K, E57T/D170E/S171E/M174Q, N141T/A184P, E57T/N60R/N141A/D170E/S171E/M174Q/E192V, N60R/D170E/M174Q, N141C/D170E/S171E, P69L/L71K/G114N/A184M, T38D/V40E, V40E/L71K/Y98R/A184M/1259V, Y32R/N60R/N141A/D170E/S171E/M174Q/E196K/D320K, Y32R/D170E/1207T/D320K, or E196K, wherein the amino acid positions are relative to the reference sequence corresponding to residues 12 to 343 of SEQ ID NO: 106, or relative to the reference sequence corresponding to SEQ ID NO: 106.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of SEQ ID NO: 218, or to a reference sequence corresponding to SEQ ID NO: 218, wherein the amino acid sequence comprises one or more substitutions relative to the reference sequence corresponding to residues 12 to 343 of SEQ ID NO: 218, or relative to the reference sequence corresponding to SEQ ID NO: 218.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of an even-numbered SEQ ID NO of SEQ ID NOs: 270-312, or to a reference sequence corresponding to an even-numbered SEQ ID NO of SEQ ID NOs: 270-312, wherein the amino acid sequence comprises one or more substitutions relative to the reference sequence corresponding to residues 12 to 343 of SEQ ID NO: 218, or relative to the reference sequence corresponding to SEQ ID NO: 218.

In some embodiments, the amino acid sequence of the engineered RNA ligases comprises a substitution or substitution set at amino acid positions(s) 17, 190, 16, 68, 23, 270, 117/229, or 117, wherein the amino acid positions are relative to the reference sequence corresponding to residues 12 to 343 of SEQ ID NO: 218, or relative to the reference sequence corresponding to SEQ ID NO: 218.

In some embodiments, the amino acid sequence of the engineered RNA ligases comprises a substitution or substitution set or amino acid residue(s) 102T, 17P, 190F, 16R, 68W, 23M, 270A, 117K/229A, 117W, 117T, 117H, 117A, 117M, 117G, 117I, 117K, 117R, 117S, 117V, 117F, 117C, or 117L, wherein the amino acid positions are relative to the reference sequence corresponding to residues 12 to 343 of SEQ ID NO: 218, or relative to the reference sequence corresponding to SEQ ID NO: 218.

In some embodiments, the amino acid sequence of the engineered RNA ligases comprises a substitution or substitution set or amino acid residue(s) A102T, S17P, A190F, Y16R, G68W, Y23M, Y270A, Q117K/T229A, Q117W, Q117T, Q117H, Q117A, Q117M, Q117G, Q117I, Q117K, Q117R, Q117S, Q117V, Q117F, Q117C, or Q117L, wherein the amino acid positions are relative to the reference sequence corresponding to residues 12 to 343 of SEQ ID NO: 218, or relative to the reference sequence corresponding to SEQ ID NO: 218.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of SEQ ID NO: 286, or to a reference sequence corresponding to SEQ ID NO: 286, wherein the amino acid sequence comprises one or more substitutions relative to the reference sequence corresponding to residues 12 to 343 of SEQ ID NO: 286, or relative to the reference sequence corresponding to SEQ ID NO: 286.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of an even-numbered SEQ ID NO of SEQ ID NOs: 314-426, or to a reference sequence corresponding to an even-numbered SEQ ID NO of SEQ ID NOs: 314-426, wherein the amino acid sequence comprises one or more substitutions relative to the reference sequence corresponding to residues 12 to 343 of SEQ ID NO: 286, or relative to the reference sequence corresponding to SEQ ID NO: 286.

In some embodiments, the amino acid sequence of the engineered RNA ligases comprises a substitution or substitution set at amino acid positions(s) 296, 114/117, 26, 30, 117/118, 117/119, 14, 183, 117, 185, 307, 12, 93/135, 135, 92, 288, 117/120, 79, 100, 287, 292, 25, 75, 327, or 111, wherein the amino acid positions are relative to the reference sequence corresponding to residues 12 to 343 of SEQ ID NO: 286, or relative to the reference sequence corresponding to SEQ ID NO: 286.

In some embodiments, the amino acid sequence of the engineered RNA ligases comprises a substitution or substitution set or amino acid residue(s) 296E, 114V/117K, 26S, 30C, 117K/118G, 114P/117K, 114S/117K, 296L, 117K/119S, 26P, 14F, 114G/117K, 114F/117K, 117K/119A, 183V, 26R, 117K, 296R, 185S, 26F, 117K/118L, 307A, 30R, 12S, 93A/135T, 117K/118Q, 185H, 135Q, 92M, 288A, 92N, 14W, 117K/119T, 92S, 92G, 117K/120P, 79W, 100R, 287A, 117K/119E, 296V, 292E, 26H, 296W, 30H, 26G, 30A, 25W, 25L, 26V, 26L, 92E, 14T, 75T, 25G, 327W, or 111L, wherein the amino acid positions are relative to the reference sequence corresponding to residues 12 to 343 of SEQ ID NO: 286, or relative to the reference sequence corresponding to SEQ ID NO: 286.

In some embodiments, the amino acid sequence of the engineered RNA ligases comprises a substitution or substitution set or amino acid residue(s) G296E, N114V/W117K, K26S, K30C, W117K/K118G, N114P/W117K, N114S/W117K, G296L, W117K/G119S, K26P, K14F, N114G/W117K, N114F/W117K, W117K/G119A, L183V, K26R, W117K, G296R, A185S, K26F, W117K/K118L, S307A, K30R, M12S, V93A/N135T, W117K/K118Q, A185H, N135Q, T92M, P288A, T92N, K14W, W117K/G119T, T92S, T92G, W117K/V120P, Y79W, A100R, T287A, W117K/G119E, G296V, G292E, K26H, G296W, K30H, K26G, K30A, S25W, S25L, K26V, K26L, T92E, K14T, D75T, S25G, V327W, or F111L, wherein the amino acid positions are relative to the reference sequence corresponding to residues 12 to 343 of SEQ ID NO: 286, or relative to the reference sequence corresponding to SEQ ID NO: 286.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of SEQ ID NO: 396, or to a reference sequence corresponding to SEQ ID NO: 396, wherein the amino acid sequence comprises one or more substitutions relative to the reference sequence corresponding to residues 12 to 343 of SEQ ID NO: 396, or relative to the reference sequence corresponding to SEQ ID NO: 396.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of an even-numbered SEQ ID NO of SEQ ID NOs: 428-488, or to a reference sequence corresponding to an even-numbered SEQ ID NO of SEQ ID NOs: 428-488, wherein the amino acid sequence comprises one or more substitutions relative to the reference sequence corresponding to residues 12 to 343 of SEQ ID NO: 396, or relative to the reference sequence corresponding to SEQ ID NO: 396.

In some embodiments, the amino acid sequence of the engineered RNA ligases comprises a substitution or substitution set at amino acid positions(s) 102/119, 75/102, 75/102/117, 75/119, 75/136/296, 75/327, 75/117/119, 119, 119/296, 14/102/119, 14/102/296, 14/75, 14/75/119, 14/75/119/327, 14/75/117/119, 14/75/117/119/296, 14/119, 14/296, 14/30/270, 14/25, 14/117, 14/117/119, 14/117/119/296, 14/117/296/327, 26/75/327, 25/75, 25/30/102, 117, 117/119, or 117/119/296, wherein the amino acid positions are relative to the reference sequence corresponding to residues 12 to 343 of SEQ ID NO: 396, or relative to the reference sequence corresponding to SEQ ID NO: 396.

In some embodiments, the amino acid sequence of the engineered RNA ligases comprises a substitution or substitution set or amino acid residue(s) 102T/119E, 75T/102T, 75T/102T/117M, 75T/119E, 75T/136A/296W, 75T/327W, 75T/117K/119E, 119E, 119E/296W, 14T/102T/119E, 14T/102T/296W, 14T/75T, 14T/75T/119E, 14T/75T/119E/327W, 14T/75T/117M/119E, 14T/75T/117M/119E/296W, 14T/119E, 14T/296W, 14T/30H/270A, 14T/25G, 14T/117K, 14T/117K/119E, 14T/117M/119E, 14T/117M/119E/296W, 14T/117M/296W/327W, 26V/75T/327W, 25L/75T, 25W/30H/102T, 117M, 117M/119E, or 117M/119E/296W, wherein the amino acid positions are relative to the reference sequence corresponding to residues 12 to 343 of SEQ ID NO: 396, or relative to the reference sequence corresponding to SEQ ID NO: 396.

In some embodiments, the amino acid sequence of the engineered RNA ligases comprises a substitution or substitution set or amino acid residue(s) A102T/G119E, D75T/A102T, D75T/A102T/W117M, D75T/G119E, D75T/T136A/G296W, D75T/V327W, D75T/W117K/G119E, G119E, G119E/G296W, K14T/A102T/G119E, K14T/A102T/G296W, K14T/D75T, K14T/D75T/G119E, K14T/D75T/G119E/V327W, K14T/D75T/W117M/G119E, K14T/D75T/W117M/G119E/G296W, K14T/G119E, K14T/G296W, K14T/K30H/Y270A, K14T/S25G, K14T/W117K, K14T/W117K/G119E, K14T/W117M/G119E, K14T/W117M/G119E/G296W, K14T/W117M/G296W/V327W, K26V/D75T/V327W, S25L/D75T, S25W/K30H/A102T, W117M, W117M/G119E, or W117M/G119E/G296W, wherein the amino acid positions are relative to the reference sequence corresponding to residues 12 to 343 of SEQ ID NO: 396, or relative to the reference sequence corresponding to SEQ ID NO: 396.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of SEQ ID NO: 436, or to a reference sequence corresponding to SEQ ID NO: 436, wherein the amino acid sequence comprises one or more substitutions relative to the reference sequence corresponding to residues 12 to 343 of SEQ ID NO: 436, or relative to the reference sequence corresponding to SEQ ID NO: 436.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of an even-numbered SEQ ID NO of SEQ ID NOs: 490-534, or to a reference sequence corresponding to an even-numbered SEQ ID NO of SEQ ID NOs: 490-534, wherein the amino acid sequence comprises one or more substitutions relative to the reference sequence corresponding to residues 12 to 343 of SEQ ID NO: 436, or relative to the reference sequence corresponding to SEQ ID NO: 436.

In some embodiments, the amino acid sequence of the engineered RNA ligases comprises a substitution or substitution set at amino acid position 332, 156, 177, 333, 96, 11, 189, 327, 25, 73, 98, 335, 95, 188, or 52, or combinations thereof, wherein the amino acid positions are relative to the reference sequence corresponding to residues 12 to 343 of SEQ ID NO: 436, or relative to the reference sequence corresponding to SEQ ID NO: 436.

In some embodiments, the amino acid sequence of the engineered RNA ligases comprises a substitution or amino acid residue 332R, 156C, 177L, 333A, 96A, 11P, 156Y, 189L, 177I, 327I, 333T, 189V, 25K, 73T, 333S, 98S, 335L, 327R, 95E, 188S, 98E, 52L, or 335K, or combinations thereof, wherein the amino acid positions are relative to the reference sequence corresponding to residues 12 to 343 of SEQ ID NO: 436, or relative to the reference sequence corresponding to SEQ ID NO: 436.

In some embodiments, the amino acid sequence of the engineered RNA ligases comprises a substitution or amino acid residue D332R, F156C, N177L, V333A, F96A, G11P, F156Y, T189L, N177I, V327I, V333T, T189V, S25K, A73T, V333S, Y98S, E335L, V327R, K95E, A188S, Y98E, F52L, or E335K, or combinations thereof, wherein the amino acid positions are relative to the reference sequence corresponding to residues 12 to 343 of SEQ ID NO: 436, or relative to the reference sequence corresponding to SEQ ID NO: 436.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of SEQ ID NO: 520, or to a reference sequence corresponding to SEQ ID NO: 520, wherein the amino acid sequence comprises one or more substitutions relative to the reference sequence corresponding to residues 12 to 343 of SEQ ID NO: 520, or relative to the reference sequence corresponding to SEQ ID NO: 520.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of an even-numbered SEQ ID NO of SEQ ID NOs: 536-554, or to a reference sequence corresponding to an even-numbered SEQ ID NO of SEQ ID NOs: 536-554, wherein the amino acid sequence comprises one or more substitutions relative to the reference sequence corresponding to residues 12 to 343 of SEQ ID NO: 520, or relative to the reference sequence corresponding to SEQ ID NO: 520.

In some embodiments, the amino acid sequence of the engineered RNA ligases comprises at least a substitution set at amino acid positions 96/98/156/189/335, 73/96/98/189/335, 73/177/189, 73/98/177/189/335, 25/73/95/96/98/189/327, 25/98/189, 73/95/96/156/177, 73/335, 73/96/98/156/335, or 73/177/189/333, wherein the amino acid positions are relative to the reference sequence corresponding to residues 12 to 343 of SEQ ID NO: 520, or relative to the reference sequence corresponding to SEQ ID NO: 520.

In some embodiments, the amino acid sequence of the engineered RNA ligases comprises at least a substitution set or amino acid residues 96S/98Y/156C/189V/335K, 73T/96S/98E/189L/335K, 73T/177L/189V, 73T/98E/177L/189V/335K, 25A/73T/95E/96A/98E/189V/327I, 25K/98Y/189V, 73T/95E/96S/156Y/177L, 73T/335K, 73T/96S/98Y/156C/335K, or 73T/177L/189V/333T, wherein the amino acid positions are relative to the reference sequence corresponding to residues 12 to 343 of SEQ ID NO: 520, or relative to the reference sequence corresponding to SEQ ID NO: 520.

In some embodiments, the amino acid sequence of the engineered RNA ligases comprises at least a substitution set or amino acid residues F96S/S98Y/F156C/T189V/E335K, A73T/F96S/S98E/T189L/E335K, A73T/N177L/T189V, A73T/S98E/N177L/T189V/E335K, S25A/A73T/K95E/F96A/S98E/T189V/V327I, S25K/S98Y/T189V, A73T/K95E/F96S/F156Y/N177L, A73T/E335K, A73T/F96S/S98Y/F156C/E335K, or A73T/N177L/T189V/V333T, wherein the amino acid positions are relative to the reference sequence corresponding to residues 12 to 343 of SEQ ID NO: 520, or relative to the reference sequence corresponding to SEQ ID NO: 520.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of SEQ ID NO: 552, or to a reference sequence corresponding to SEQ ID NO: 552, wherein the amino acid sequence comprises one or more substitutions relative to the reference sequence corresponding to residues 12 to 343 of SEQ ID NO: 552, or relative to the reference sequence corresponding to SEQ ID NO: 552.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of an even-numbered SEQ ID NO of SEQ ID NOs: 556-584, or to a reference sequence corresponding to an even-numbered SEQ ID NO of SEQ ID NOs: 556-584, wherein the amino acid sequence comprises one or more substitutions relative to the reference sequence corresponding to residues 12 to 343 of SEQ ID NO: 552, or relative to the reference sequence corresponding to SEQ ID NO: 552.

In some embodiments, the amino acid sequence of the engineered RNA ligases comprises at least a substitution at amino acid position 292, 147, 255, 295, 75, 300, 333, 200, 289, 95, or 168, or combinations thereof, wherein the amino acid positions are relative to the reference sequence corresponding to residues 12 to 343 of SEQ ID NO: 552, or relative to the reference sequence corresponding to SEQ ID NO: 552.

In some embodiments, the amino acid sequence of the engineered RNA ligases comprises at least a substitution or amino acid residue 292Y, 147L, 255S, 295R, 75L, 300E, 333E, 200S, 289S, 95V, 289A, 289T, 168A, 289H, or 200T, wherein the amino acid positions are relative to the reference sequence corresponding to residues 12 to 343 of SEQ ID NO: 552, or relative to the reference sequence corresponding to SEQ ID NO: 552.

In some embodiments, the amino acid sequence of the engineered RNA ligases comprises at least a substitution or amino acid residue E292Y, Y147L, P255S, M295R, T75L, Q300E, V333E, C200S, K289S, K95V, K289A, K289T, T168A, K289H, or C200T, wherein the amino acid positions are relative to the reference sequence corresponding to residues 12 to 343 of SEQ ID NO: 552, or relative to the reference sequence corresponding to SEQ ID NO: 552.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of SEQ ID NO: 582, or to a reference sequence corresponding to SEQ ID NO: 582, wherein the amino acid sequence comprises one or more substitutions relative to the reference sequence corresponding to residues 12 to 343 of SEQ ID NO: 582, or relative to the reference sequence corresponding to SEQ ID NO: 582.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of an even-numbered SEQ ID NO of SEQ ID NOs: 586-958, or to a reference sequence corresponding to an even-numbered SEQ ID NO of SEQ ID NOs: 586-958, wherein the amino acid sequence comprises one or more substitutions relative to the reference sequence corresponding to residues 12 to 343 of SEQ ID NO: 582, or relative to the reference sequence corresponding to SEQ ID NO: 582.

In some embodiments, the amino acid sequence of the engineered RNA ligases comprises at least a substitution set at amino acid position(s) 95/156/337, 189/200/271, 200/271/289/337, 200/271/289, 189/200, 156/200/289, 156/168/189/271/300, 156/189/200/289, 156/168/289, 95/156/271/289, 156/333, 95/200/289/337, 168/200/289, 189/200/289, 333/337, 103/156/271, 95/156/200/300/333, 156/271/289/333, 95/156/271, 95/189/289, 95/156/168/189/271/289/300, 189/271/289, 95/168/271/300, 156/189/289/337, 200/271, 156/189/333, 189/289, 156/189/200/300/333/337, 156/189/200/271/289/337, 156/200/271/289/333/337, 168/271/300, 289/337, 156/168/337, 156/168/189/289, 156/271/289, 189/300, 156/289/300/337, 168/189/289, 95/156/168/300, 156/200/300/337, 289/333/337, 95/189/200/300/337, 95/156/189/200/271/333, 200/271/289/300, 289/333, 189/271/300, 95/300, 95/189/333/337, 95/189/271/300, 95/189/200/289, 168, 156/200/271/289/337, 95/168/289, 156/289, 95/156/189/271/289, 156/168/189/200/289/333, 156/300/333/337, 95/156/189/200/271/337, 271, 156/271, 156/271/300/333, 95/168/200, 168/271, 95/200/333, 189/200/271/289/333, 95/168/189/333/337, 189, 189/333/337, 156/189/271/289/333, 289, 95/156/200/271/289, 289/300/333, 95/189/289/337, 95/156/168/189/289, 271/289, 333, 168/337, 156/189/271/289, 95/156/189/337, 156/189/271/300, 156/168/289/333, 168/189/289/333, 95/333, 333/343, 156/189/289, 95/289/300, 95/156/189/200/300/333/337, 189/200/271/289/337, 95/156/168/189/289/337, 95/289, 156/289/333/337, 156/289/337, 337, 168/289, 95/156/300, 95/156/289, 95/189/271/333/337, 95/168/300, 95/156/168/271/333/337, 95/189/289/333/337, 156/168/200/289/334, 95/156/168/189/271/289, 156/189/200/289/337, 200/289, 200/289/333/337, 95/168/189/200/289, 168/200, 156/168/271/289, 189/206/289, 156/200/289/333/335, 156/200/289/333, 189/200/289/333, 189/289/335, 189/254/289/333, 167/189/206/289/333/335, 156/200/333, 156/200/289/335, 156/189/289/335, 335, 156/167/189/289, 156/189/200/289/335, 200/289/333/335, 156/189/200/289/333, 254/333/335, 189/254/333/335, 189/335, 156/189/254/289, 156/289/333, 289/335, 189/200/254/289/333, 156/189/289/333, 25/156/189/289, 156/189/206/289/333/335, 289/333/335, 156/254/289/333, 156/289/333/335, 254/289, 200/289/333, 156/189/200, 156/254/333/335, 156, 254/289/335, 156/206/289, 156/289/335, 206/289/333/335, 156/189/254/289/333, 156/254/289, 200/289/335, 156/189/206/289, 156/189/200/206/254/289/333, 25/254/333, 189/289/333/335, 156/189/200/289/333/335, 254/289/333, or 156/200/333/335, wherein the amino acid positions are relative to the reference sequence corresponding to residues 12 to 343 of SEQ ID NO: 582, or relative to the reference sequence corresponding to SEQ ID NO: 582.

In some embodiments, the amino acid sequence of the engineered RNA ligases comprises at least a substitution or substitution set or amino acid residue(s) 95V/156Y/337G, 189V/200T/271I, 200S/271I/289A/337G, 200T/271I/289A, 189V/200S, 156Y/200S/289T, 156F/168A/189V/271I/300E, 156Y/189V/200T/289A, 156F/168A/289K, 95V/156Y/271I/289K, 156F/333E, 95V/200T/289K/337G, 168A/200S/289A, 189V/200T/289K, 333E/337G, 103I/156F/271I, 95V/156F/200S/300E/333E, 156F/271I/289T/333E, 95V/156F/271I, 95V/189V/289K, 95V/156Y/168A/189V/271I/289T/300E, 189V/271I/289A, 95V/168A/271I/300E, 156Y/189V/289A/337G, 200S/271I, 156F/189V/333E, 189V/289T, 156Y/189V/200T/300E/333E/337G, 156Y/200T/289A, 156F/189V/200S/271I/289K/337G, 156Y/200S/271I/289A/333E/337G, 168A/271I/300E, 289T/337G, 156Y/168A/337G, 156F/168A/189V/289T, 156Y/271I/289T, 189V/300E, 156Y/289T/300E/337G, 168A/189V/289T, 95V/156F/168A/300E, 156Y/200S/300E/337G, 289A/333E/337G, 95V/189V/200T/300E/337G, 95V/156F/189V/200T/271I/333E, 200S/271I/289A/300E, 289A/333E, 189V/271I/300E, 95V/300E, 95V/189V/333E/337G, 95V/189V/271I/300E, 95V/189V/200S/289A, 168A, 156Y/200S/271I/289K/337G, 95V/168A/289T, 156F/289A, 95V/156Y/189V/271I/289K, 156F/168A/189V/200S/289A/333E, 156F/300E/333E/337G, 95V/156F/189V/200S/271I/337G, 156F/189V/200T/289K, 156Y/289T, 271I, 156Y/271I, 156F/271I/300E/333E, 95V/168A/200S, 189V/200S/289A, 168A/271I, 95V/200T/333E, 189V/200S/271I/289A/333E, 95V/168A/189V/333E/337G, 189V, 189V/333E/337G, 156F/189V/271I/289A/333E, 289A, 95V/156F/200T/271I/289K, 289A/300E/333E, 95V/189V/289A/337G, 95V/156F/168A/189V/289A, 271I/289T, 333E, 168A/337G, 156Y/289A, 156Y/189V/271I/289K, 95V/156F/189V/337G, 189V/289K, 189V/289A, 156Y/189V/271I/300E, 156F/168A/289A/333E, 168A/189V/289K/333E, 95V/333E, 333E/343P, 156F/189V/289A, 95V/289A/300E, 95V/156Y/189V/200S/300E/333E/337G, 189V/200T/271I/289K/337G, 95V/156Y/168A/189V/289K/337G, 156F/289K, 95V/289A, 156Y/168A/289A, 156F/289K/333E/337G, 156F/289K/337G, 289A/337G, 337G, 168A/289A, 95V/156F/300E, 95V/156Y/289K, 95V/189V/271I/333E/337G, 95V/168A/300E, 95V/156Y/168A/271I/333E/337G, 95V/189V/289A/333E/337G, 156F/168A/200S/289A/334S, 95V/156F/168A/189V/271I/289T, 156Y/189V/200T/289T/337G, 156F/271I, 289K/300E/333E, 95V/156Y/271I, 200S/289A, 200S/289K/333E/337G, 95V/168A/189V/200T/289A, 156Y/271I/289T/333E, 168A/200S, 156F/168A/271I/289A, 189L/206R/289S, 156F/200S/289S/333E/335E, 156F/200S/289S/333E, 189V/200S/289K/333E, 189L/289K/335E, 189V/254G/289S/333E, 189L/289S, 167A/189L/206R/289S/333E/335E, 156F/200S/333E, 189L/200S/289S, 156Y/200S/289K/335E, 156F/189L/200S/289K, 156Y/189L/289K/335E, 156Y/200S/289K/333E, 335E, 156F/200S/289K/333E, 156F/167A/189V/289T, 156Y/189L/200S/289S/335E, 200S/289S/333E/335E, 156Y/189V/200S/289T/333E, 254G/333E/335E, 189L/254G/333E/335E, 289S, 189V/289S, 189V/335E, 156F/289T, 156F/189V/254G/289S, 156Y/289K/333E, 289S/335E, 189L/200S/254G/289S/333E, 156F/189L/289S/333E, 156Y/189V/289S/335E, 25A/156Y/189L/289S, 156Y/189V/206R/289K/333E/335E, 289S/333E/335E, 189V/200S/289S, 156F/254G/289K/333E, 156Y/289T/333E/335E, 254G/289T, 200S/289T/333E, 156Y/189V/200S, 156Y/254G/333E/335E, 156F, 254G/289K/335E, 200S/289T, 200S/289S, 156Y/206R/289S, 156F/289S/335E, 206R/289T/333E/335E, 156F/189V/254G/289K/333E, 156F/254G/289T, 200S/289T/335E, 156Y/189V/206R/289S, 156Y/189V/200S/206R/254G/289T/333E, 156Y/333E, 156Y/189L/254G/289T/333E, 25A/254G/333E, 189L/289S/333E/335E, 200S/289K/335E, 156Y/189V/289S, 156F/189L/200S/289K/333E/335E, 289T, 156F/200S/289T/335E, 254G/289S/333E, or 156F/200S/333E/335E, wherein the amino acid positions are relative to the reference sequence corresponding to residues 12 to 343 of SEQ ID NO: 582, or relative to the reference sequence corresponding to SEQ ID NO: 582.

In some embodiments, the amino acid sequence of the engineered RNA ligases comprises at least a substitution or substitution set or amino acid residue(s) K95V/C156Y/A337G, T189V/C200T/V271I, C200S/V271I/H289A/A337G, C200T/V271I/H289A, T189V/C200S, C156Y/C200S/H289T, C156F/T168A/T189V/V271I/Q300E, C156Y/T189V/C200T/H289A, C156F/T168A/H289K, K95V/C156Y/V271I/H289K, C156F/V333E, K95V/C200T/H289K/A337G, T168A/C200S/H289A, T189V/C200T/H289K, V333E/A337G, V103I/C156F/V271I, K95V/C156F/C200S/Q300E/V333E, C156F/V271I/H289T/V333E, K95V/C156F/V271I, K95V/T189V/H289K, K95V/C156Y/T168A/T189V/V271I/H289T/Q300E, T189V/V271I/H289A, K95V/T168A/V271I/Q300E, C156Y/T189V/H289A/A337G, C200S/V271I, C156F/T189V/V333E, T189V/H289T, C156Y/T189V/C200T/Q300E/V333E/A337G, C156Y/C200T/H289A, C156F/T189V/C200S/V271I/H289K/A337G, C156Y/C200S/V271I/H289A/V333E/A337G, T168A/V271I/Q300E, H289T/A337G, C156Y/T168A/A337G, C156F/T168A/T189V/H289T, C156Y/V271I/H289T, T189V/Q300E, C156Y/H289T/Q300E/A337G, T168A/T189V/H289T, K95V/C156F/T168A/Q300E, C156Y/C200S/Q300E/A337G, H289A/V333E/A337G, K95V/T189V/C200T/Q300E/A337G, K95V/C156F/T189V/C200T/V271I/V333E, C200S/V271I/H289A/Q300E, H289A/V333E, T189V/V271I/Q300E, K95V/Q300E, K95V/T189V/V333E/A337G, K95V/T189V/V271I/Q300E, K95V/T189V/C200S/H289A, T168A, C156Y/C200S/V271I/H289K/A337G, K95V/T168A/H289T, C156F/H289A, K95V/C156Y/T189V/V271I/H289K, C156F/T168A/T189V/C200S/H289A/V333E, C156F/Q300E/V333E/A337G, K95V/C156F/T189V/C200S/V271I/A337G, C156F/T189V/C200T/H289K, C156Y/H289T, V271I, C156Y/V271I, C156F/V271I/Q300E/V333E, K95V/T168A/C200S, T189V/C200S/H289A, T168A/V271I, K95V/C200T/V333E, T189V/C200S/V271I/H289A/V333E, K95V/T168A/T189V/V333E/A337G, T189V, T189V/V333E/A337G, C156F/T189V/V271I/H289A/V333E, H289A, K95V/C156F/C200T/V271I/H289K, H289A/Q300E/V333E, K95V/T189V/H289A/A337G, K95V/C156F/T168A/T189V/H289A, V271I/H289T, V333E, T168A/A337G, C156Y/H289A, C156Y/T189V/V271I/H289K, K95V/C156F/T189V/A337G, T189V/H289K, T189V/H289A, C156Y/T189V/V271I/Q300E, C156F/T168A/H289A/V333E, T168A/T189V/H289K/V333E, K95V/V333E, V333E/S343P, C156F/T189V/H289A, K95V/H289A/Q300E, K95V/C156Y/T189V/C200S/Q300E/V333E/A337G, T189V/C200T/V271I/H289K/A337G, K95V/C156Y/T168A/T189V/H289K/A337G, C156F/H289K, K95V/H289A, C156Y/T168A/H289A, C156F/H289K/V333E/A337G, C156F/H289K/A337G, H289A/A337G, A337G, T168A/H289A, K95V/C156F/Q300E, K95V/C156Y/H289K, K95V/T189V/V271I/V333E/A337G, K95V/T168A/Q300E, K95V/C156Y/T168A/V271I/V333E/A337G, K95V/T189V/H289A/V333E/A337G, C156F/T168A/C200S/H289A/L334S, K95V/C156F/T168A/T189V/V271I/H289T, C156Y/T189V/C200T/H289T/A337G, C156F/V271I, H289K/Q300E/V333E, K95V/C156Y/V271I, C200S/H289A, C200S/H289K/V333E/A337G, K95V/T168A/T189V/C200T/H289A, C156Y/V271I/H289T/V333E, T168A/C200S, C156F/T168A/V271I/H289A, T189L/V206R/H289S, C156F/C200S/H289S/V333E/K335E, C156F/C200S/H289S/V333E, T189V/C200S/H289K/V333E, T189L/H289K/K335E, T189V/V254G/H289S/V333E, T189L/H289S, G167A/T189L/V206R/H289S/V333E/K335E, C156F/C200S/V333E, T189L/C200S/H289S, C156Y/C200S/H289K/K335E, C156F/T189L/C200S/H289K, C156Y/T189L/H289K/K335E, C156Y/C200S/H289K/V333E, K335E, C156F/C200S/H289K/V333E, C156F/G167A/T189V/H289T, C156Y/T189L/C200S/H289S/K335E, C200S/H289S/V333E/K335E, C156Y/T189V/C200S/H289T/V333E, V254G/V333E/K335E, T189L/V254G/V333E/K335E, H289S, T189V/H289S, T189V/K335E, C156F/H289T, C156F/T189V/V254G/H289S, C156Y/H289K/V333E, H289S/K335E, T189L/C200S/V254G/H289S/V333E, C156F/T189L/H289S/V333E, C156Y/T189V/H289S/K335E, S25A/C156Y/T189L/H289S, C156Y/T189V/V206R/H289K/V333E/K335E, H289S/V333E/K335E, T189V/C200S/H289S, C156F/V254G/H289K/V333E, C156Y/H289T/V333E/K335E, V254G/H289T, C200S/H289T/V333E, C156Y/T189V/C200S, C156Y/V254G/V333E/K335E, C156F, V254G/H289K/K335E, C200S/H289T, C200S/H289S, C156Y/V206R/H289S, C156F/H289S/K335E, V206R/H289T/V333E/K335E, C156F/T189V/V254G/H289K/V333E, C156F/V254G/H289T, C200S/H289T/K335E, C156Y/T189V/V206R/H289S, C156Y/T189V/C200S/V206R/V254G/H289T/V333E, C156Y/V333E, C156Y/T189L/V254G/H289T/V333E, S25A/V254G/V333E, T189L/H289S/V333E/K335E, C200S/H289K/K335E, C156Y/T189V/H289S, C156F/T189L/C200S/H289K/V333E/K335E, H289T, C156F/C200S/H289T/K335E, V254G/H289S/V333E, or C156F/C200S/V333E/K335E, wherein the amino acid positions are relative to the reference sequence corresponding to residues 12 to 343 of SEQ ID NO: 582, or relative to the reference sequence corresponding to SEQ ID NO: 582.

In some embodiments, the amino acid sequence of the engineered RNA ligase comprises at least a substitution at an amino acid position set forth in Tables 3, 4, 5, 6, 7.1, 7.2, 8, 9, 10, 11.3, 11.4, and 12, wherein the amino acid positions are relative to the reference sequence corresponding to residues 12 to 343 of SEQ ID NO: 2, 4, 106, 218, 286, 396, 436, 520, 552, or 582, or relative to the reference sequence corresponding to SEQ ID NO: 2, 4, 106, 218, 286, 396, 436, 520, 552, or 582.

In some embodiments, the amino acid sequence of the engineered RNA ligase comprises at least one substitution set forth in Tables 3, 4, 5, 6, 7.1, 7.2, 8, 9, 10, 11.3, 11.4, and 12, wherein the amino acid positions are relative to the reference sequence corresponding to residues 12 to 343 of SEQ ID NO: 2, 4, 106, 218, 286, 396, 436, 520, 552, or 582, or relative to the reference sequence corresponding to SEQ ID NO: 2, 4, 106, 218, 286, 396, 436, 520, 552, or 582.

In some embodiments, the amino acid sequence of the engineered RNA ligase comprises at least a substitution or substitution set at amino acid position(s) set forth in Tables 3, 4, 5, 6, 7.1, 7.2, 8, 9, 10, 11.3, 11.4, and 12, wherein the amino acid positions are relative to the reference sequence corresponding to residues 12 to 343 of SEQ ID NO: 2, 4, 106, 218, 286, 396, 436, 520, 552, or 582, or relative to the reference sequence corresponding to SEQ ID NO: 2, 4, 106, 218, 286, 396, 436, 520, 552, or 582.

In some embodiments, the amino acid sequence of the engineered RNA ligase comprises at least a substitution or substitution set of an RNA ligase variant set forth in Tables 3, 4, 5, 6, 7.1, 7.2, 8, 9, 10, 11.3, 11.4, and 12, wherein the amino acid positions are relative to the reference sequence corresponding to residues 12 to 343 of SEQ ID NO: 2, 4, 106, 218, 286, 396, 436, 520, 552, or 582, or relative to the reference sequence corresponding to SEQ ID NO: 2, 4, 106, 218, 286, 396, 436, 520, 552, or 582.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference sequence comprising a substitution or substitution set of an RNA ligase variant set forth in Tables 3, 4, 5, 6, 7.1, 7.2, 8, 9, 10, 11.3, 11.4, and 12, wherein the amino acid positions are relative to the reference sequence corresponding to residues 12 to 343 of SEQ ID NO: 2, 4, 106, 218, 286, 396, 436, 520, 552, or 582, or relative to the reference sequence corresponding to SEQ ID NO: 2, 4, 106, 218, 286, 396, 436, 520, 552, or 582.

In some embodiments, the engineered RNA ligase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference sequence corresponding to residues 12 to 343 of SEQ ID NO: 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 224, 226, 228, 230, 232, 234, 236, 238, 240, 242, 244, 246, 248, 250, 252, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 506, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, 726, 728, 730, 732, 734, 736, 738, 740, 742, 744, 746, 748, 750, 752, 754, 756, 758, 760, 762, 764, 766, 768, 770, 772, 774, 776, 778, 780, 782, 784, 786, 788, 790, 792, 794, 796, 798, 800, 802, 804, 806, 808, 810, 812, 814, 816, 818, 820, 822, 824, 826, 828, 830, 832, 834, 836, 838, 840, 842, 844, 846, 848, 850, 852, 854, 856, 858, 860, 862, 864, 866, 868, 870, 872, 874, 876, 878, 880, 882, 884, 886, 888, 890, 892, 894, 896, 898, 900, 902, 904, 906, 908, 910, 912, 914, 916, 918, 920, 922, 924, 926, 928, 930, 932, 934, 936, 938, 940, 942, 944, 946, 948, 950, 952, 954, 956, or 958.

In some embodiments, the engineered RNA ligase comprises an amino acid sequence having at least 70%, 75%, 80%, 85%, 86%, 87%, 88%, 89%, 90%, 91%, 92%, 93%, 94%, 95%, 96%, 97%, 98%, 99% or more sequence identity to a reference sequence corresponding to SEQ ID NO: 4, 6, 8, 10, 12, 14, 16, 18, 20, 22, 24, 26, 28, 30, 32, 34, 336, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 224, 226, 228, 230, 232, 234, 236, 238, 240, 242, 244, 246, 248, 250, 252, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 506, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, 726, 728, 730, 732, 734, 736, 738, 740, 742, 744, 746, 748, 750, 752, 754, 756, 758, 760, 762, 764, 766, 768, 770, 772, 774, 776, 778, 780, 782, 784, 786, 788, 790, 792, 794, 796, 798, 800, 802, 804, 806, 808, 810, 812, 814, 816, 818, 820, 822, 824, 826, 828, 830, 832, 834, 836, 838, 840, 842, 844, 846, 848, 850, 852, 854, 856, 858, 860, 862, 864, 866, 868, 870, 872, 874, 876, 878, 880, 882, 884, 886, 888, 890, 892, 894, 896, 898, 900, 902, 904, 906, 908, 910, 912, 914, 916, 918, 920, 922, 924, 926, 928, 930, 932, 934, 936, 938, 940, 942, 944, 946, 948, 950, 952, 954, 956, or 958.

In some embodiments, the engineered RNA ligase comprises an amino acid sequence comprising residues 12 to 343 of an even numbered SEQ ID NO. of SEQ ID NOs: 4-220, 224-252, and 270-958, or an amino acid sequence comprising an even numbered SEQ ID NO. of SEQ ID NOs: 4-220, 224-252, and 270-958. In some embodiments, the amino acid sequence of the engineered RNA ligase optionally includes 1, 2, 3, 4, 5, 6, 7, 8, 9, or up to 10 substitutions, insertions, and/or deletions. In some embodiments, the amino acid sequence of the engineered RNA ligase optionally includes 1, 2, 3, 4, 5, 6, 7, 8, 9, or up to 10 substitutions. In some embodiments, the amino acid sequence of the engineered RNA ligase optionally includes 1, 2, 3, 4, or 5 substitutions, insertions, and/or deletions. In some embodiments, the amino acid sequence of the engineered RNA ligase optionally includes 1, 2, 3, 4, or 5 substitutions.

In some embodiments, the engineered RNA ligase comprises an amino acid sequence comprising residues 12 to 343 of SEQ ID NO: 4, 6, 8, 10, 12, 14, 16, 18, 20, 22, 24, 26, 28, 30, 32, 34, 336, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 224, 226, 228, 230, 232, 234, 236, 238, 240, 242, 244, 246, 248, 250, 252, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 506, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, 726, 728, 730, 732, 734, 736, 738, 740, 742, 744, 746, 748, 750, 752, 754, 756, 758, 760, 762, 764, 766, 768, 770, 772, 774, 776, 778, 780, 782, 784, 786, 788, 790, 792, 794, 796, 798, 800, 802, 804, 806, 808, 810, 812, 814, 816, 818, 820, 822, 824, 826, 828, 830, 832, 834, 836, 838, 840, 842, 844, 846, 848, 850, 852, 854, 856, 858, 860, 862, 864, 866, 868, 870, 872, 874, 876, 878, 880, 882, 884, 886, 888, 890, 892, 894, 896, 898, 900, 902, 904, 906, 908, 910, 912, 914, 916, 918, 920, 922, 924, 926, 928, 930, 932, 934, 936, 938, 940, 942, 944, 946, 948, 950, 952, 954, 956, or 958. In some embodiments, the amino acid sequence of the engineered RNA ligase optionally includes 1, 2, 3, 4, 5, 6, 7, 8, 9, or up to 10 substitutions, insertions, and/or deletions. In some embodiments, the amino acid sequence of the engineered RNA ligase optionally includes 1, 2, 3, 4, 5, 6, 7, 8, 9, or up to 10 substitutions. In some embodiments, the amino acid sequence of the engineered RNA ligase optionally includes 1, 2, 3, 4, or 5 substitutions, insertions, and/or deletions. In some embodiments, the amino acid sequence of the engineered RNA ligase optionally includes 1, 2, 3, 4, or 5 substitutions.

In some embodiments, the engineered RNA ligase comprises an amino acid sequence comprising SEQ ID NO: 4, 6, 8, 10, 12, 14, 16, 18, 20, 22, 24, 26, 28, 30, 32, 34, 336, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 150, 152, 154, 156, 158, 160, 162, 164, 166, 168, 170, 172, 174, 176, 178, 180, 182, 184, 186, 188, 190, 192, 194, 196, 198, 200, 202, 204, 206, 208, 210, 212, 214, 216, 218, 220, 224, 226, 228, 230, 232, 234, 236, 238, 240, 242, 244, 246, 248, 250, 252, 270, 272, 274, 276, 278, 280, 282, 284, 286, 288, 290, 292, 294, 296, 298, 300, 302, 304, 306, 308, 310, 312, 314, 316, 318, 320, 322, 324, 326, 328, 330, 332, 334, 336, 338, 340, 342, 344, 346, 348, 350, 352, 354, 356, 358, 360, 362, 364, 366, 368, 370, 372, 374, 376, 378, 380, 382, 384, 386, 388, 390, 392, 394, 396, 398, 400, 402, 404, 406, 408, 410, 412, 414, 416, 418, 420, 422, 424, 426, 428, 430, 432, 434, 436, 438, 440, 442, 444, 446, 448, 450, 452, 454, 456, 458, 460, 462, 464, 466, 468, 470, 472, 474, 476, 478, 480, 482, 484, 486, 488, 490, 492, 494, 496, 498, 500, 502, 504, 506, 508, 510, 512, 514, 516, 518, 520, 522, 524, 526, 528, 530, 532, 534, 536, 538, 540, 542, 544, 546, 548, 550, 552, 554, 556, 558, 560, 562, 564, 566, 568, 570, 572, 574, 576, 578, 580, 582, 584, 586, 588, 590, 592, 594, 596, 598, 600, 602, 604, 506, 608, 610, 612, 614, 616, 618, 620, 622, 624, 626, 628, 630, 632, 634, 636, 638, 640, 642, 644, 646, 648, 650, 652, 654, 656, 658, 660, 662, 664, 666, 668, 670, 672, 674, 676, 678, 680, 682, 684, 686, 688, 690, 692, 694, 696, 698, 700, 702, 704, 706, 708, 710, 712, 714, 716, 718, 720, 722, 724, 726, 728, 730, 732, 734, 736, 738, 740, 742, 744, 746, 748, 750, 752, 754, 756, 758, 760, 762, 764, 766, 768, 770, 772, 774, 776, 778, 780, 782, 784, 786, 788, 790, 792, 794, 796, 798, 800, 802, 804, 806, 808, 810, 812, 814, 816, 818, 820, 822, 824, 826, 828, 830, 832, 834, 836, 838, 840, 842, 844, 846, 848, 850, 852, 854, 856, 858, 860, 862, 864, 866, 868, 870, 872, 874, 876, 878, 880, 882, 884, 886, 888, 890, 892, 894, 896, 898, 900, 902, 904, 906, 908, 910, 912, 914, 916, 918, 920, 922, 924, 926, 928, 930, 932, 934, 936, 938, 940, 942, 944, 946, 948, 950, 952, 954, 956, or 958. In some embodiments, the amino acid sequence of the engineered RNA ligase optionally includes 1, 2, 3, 4, 5, 6, 7, 8, 9, or up to 10 substitutions, insertions, and/or deletions. In some embodiments, the amino acid sequence of the engineered RNA ligase optionally includes 1, 2, 3, 4, 5, 6, 7, 8, 9, or up to 10 substitutions. In some embodiments, the amino acid sequence of the engineered RNA ligase optionally includes 1, 2, 3, 4, or 5 substitutions, insertions, and/or deletions. In some embodiments, the amino acid sequence of the engineered RNA ligase optionally includes 1, 2, 3, 4, or 5 substitutions.

In some embodiments, the engineered RNA ligase comprises an amino acid sequence comprising residues 12 to 343 of SEQ ID NO: 2, 106, 218, 286, 396, 436, 520, 552, or 582, or an amino acid sequence comprising SEQ ID NO: 2, 106, 218, 286, 396, 436, 520, 552, or 582. In some embodiments, the amino acid sequence of the engineered RNA ligase optionally includes 1, 2, 3, 4, 5, 6, 7, 8, 9, or up to 10 substitutions, insertions, and/or deletions. In some embodiments, the amino acid sequence of the engineered RNA ligase optionally includes 1, 2, 3, 4, 5, 6, 7, 8, 9, or up to 10 substitutions. In some embodiments, the amino acid sequence of the engineered RNA ligase optionally includes 1, 2, 3, 4, or 5 substitutions, insertions, and/or deletions. In some embodiments, the amino acid sequence of the engineered RNA ligase optionally includes 1, 2, 3, 4, or 5 substitutions.

In some embodiments, the engineered RNA ligase of the present disclosure has RNA ligase activity on single stranded and/or double stranded polynucleotide substrates. In some embodiments, the engineered RNA ligase has RNA ligase activity and one or more improved or enhanced property as compared to a reference RNA ligase. Exemplary improved properties are provided in the Examples.

In some embodiments, the engineered RNA ligase has increased activity as compared to the reference RNA ligase. In some embodiments, the engineered RNA ligase has 1.1, 1.2, 1.3, 1.4, 1.5, 2, 2.5, 3, 3.5, 4, 4.5, 5, 6, 7, 8, 9, 10 or more fold activity compared to the reference RNA ligase. In some embodiments, the assay conditions for measuring ligase activity is described in the Examples. In some embodiments, the increase in activity is on a double stranded nick formed by double stranded fragments that have complementary ends and base pair to form the double stranded nicks, particularly where at least one or both strands of the double stranded fragments comprise modified nucleotides.

In some embodiments, the engineered RNA ligase has increased product yield as compared to the reference RNA ligase. In some embodiments, the engineered RNA ligase has increased product yield on polynucleotides substrates with phosphorothioate internucleoside linkages as compared to the reference RNA ligase. In some embodiments, the engineered RNA ligase has increased product yield on polynucleotides substrates with 2′-modifications (e.g., 2′-O-methyl and/or 2′-fluoro) as compared to the reference RNA ligase.

In particular, the engineered RNA ligase has increased activity on polynucleotide substrates having a 2′-modified sugar moiety on the 3′-terminal nucleotide and/or 5′-terminal nucleotide, where the 3′-terminal nucleotide on the polynucleotide acceptor strand is being ligated to the 5′-terminal nucleotide of the polynucleotide donor strand substrates.

In some embodiments, the engineered RNA ligase exhibits for double stranded ligase substrates a efficiency of at least 60%, 70%, 80%, 90%, 91%, 92%, 93%, 94%, 95%, 96%, 97%, 98%, 99%, or greater on polynucleotide acceptor substrate comprising a modified nucleoside at the 3′-terminal nucleoside of the polynucleotide acceptor strand. In some embodiments, the engineered RNA ligase exhibits a ligation efficiency of at least 60%, 70%, 80%, 90%, 91%, 92%, 93%, 94%, 95%, 96%, 97%, 98%, 99%, or greater for a polynucleotide acceptor strand having a modified 3′-terminal nucleoside of 2′-fluoro-adenosine, 2′-fluoro-guanosine, 2′-fluoro-cytidine, 2′-fluoro-uridine, 2′-fluoro-uridine, 2′-O-methyl-adenosine, 2′-O-methyl-guanosine, 2′-O-methyl-cytidine, or 2′-O-methyl-uridine.

In some embodiments, the engineered RNA ligase exhibits for double stranded ligase substrates a efficiency of at least 60%, 70%, 80%, 90%, 91%, 92%, 93%, 94%, 95%, 96%, 97%, 98%, 99%, or greater on polynucleotide donor substrate comprising a modified nucleoside at the 5′-terminal nucleoside of the polynucleotide acceptor strand. In some embodiments, the engineered RNA ligase exhibits a ligation efficiency of at least 60%, 70%, 80%, 90%, 91%, 92%, 93%, 94%, 95%, 96%, 97%, 98%, 99%, or greater for a polynucleotide donor strand having a modified 5′-terminal nucleoside of 2′-fluoro-adenosine, 2′-fluoro-guanosine, 2′-fluoro-cytidine, 2′-fluoro-uridine, 2′-fluoro-uridine, 2′-O-methyl-adenosine, 2′-O-methyl-guanosine, 2′-O-methyl-cytidine, or 2′-O-methyl-uridine.

In some embodiments, the engineered RNA ligase exhibits for double stranded ligase substrates a efficiency of at least 60%, 70%, 80%, 90%, 91%, 92%, 93%, 94%, 95%, 96%, 97%, 98%, 99%, or greater on polynucleotide substrates comprising a modified nucleoside at the 3′-terminal nucleoside of the polynucleotide acceptor strand. In some embodiments, the engineered RNA ligase exhibits a ligation efficiency of at least 60%, 70%, 80%, 90%, 91%, 92%, 93%, 94%, 95%, 96%, 97%, 98%, 99%, or greater for ligation of a ligase substrate comprising:
a polynucleotide acceptor strand having a modified 3′-terminal nucleoside of 2′-fluoro-adenosine, 2′-fluoro-guanosine, 2′-fluoro-cytidine, 2′-fluoro-uridine, 2′-fluoro-uridine, 2′-O-methyl-adenosine, 2′-O-methyl-guanosine, 2′-O-methyl-cytidine, or 2′-O-methyl-uridine;
a polynucleotide donor strand having a modified 5′-terminal nucleoside of 2′-fluoro-adenosine, 2′-fluoro-guanosine, 2′-fluoro-cytidine, 2′-fluoro-uridine, 2′-fluoro-uridine, 2′-O-methyl-adenosine, 2′-O-methyl-guanosine, 2′-O-methyl-cytidine, or 2′-O-methyl-uridine; and
a polynucleotide complementary to the polynucleotide acceptor strand and polynucleotide donor strand, wherein the polynucleotide complementary to the polynucleotide acceptor strand and polynucleotide donor strand comprises a complementary 2′-fluoro or 2′-O-methyl nucleoside at the position complementary to the 3′-terminal nucleoside of the polynucleotide acceptor strand, and/or a complementary 2-fluoro or 2-O-methyl nucleoside at the position complementary to the 5′-terminal nucleoside of the polynucleotide donor strand.

In some embodiments, the reference RNA ligase for comparing an RNA ligase property has the sequence corresponding to residues 12 to 343 of SEQ ID NO: 2, 4, 106, 218, 286, 396, 436, 520, 552, or 582, or the sequence corresponding to SEQ ID NO: 2, 4, 106, 218, 286, 396, 436, 520, 552, or 582. In some embodiments, the reference RNA ligase has the sequence corresponding to residues 12 to 343 of SEQ ID NO: 2, 4, 106, 218, 286, 396, 436, 520, 552, or 582, or the sequence corresponding to SEQ ID NO: 2, 4, 106, 218, 286, 396, 436, 520, 552, or 582.

In some embodiments, the engineered RNA ligase has one or more improved property selected from i) increased activity, ii) increased product yield, iii) increased product yield on polynucleotides with phosphorothioate internucleoside linkages, iv) increased product yield on oligonucleotides with 2′-modifications, and v) increased expression, or any combination of i), ii), iii), iv), and v) compared to a reference RNA ligase. In some embodiments, the reference RNA ligase has the sequence corresponding to residues 12 to 343 of SEQ ID NO: 2, 4, 106, 218, 286, 396, 436, 520, 552, or 582, or the sequence corresponding to SEQ ID NO: 2, 4, 106, 218, 286, 396, 436, 520, 552, or 582. In some embodiments, the reference RNA ligase has the sequence corresponding to residues 12 to 343 of SEQ ID NO: 2, or the sequence corresponding to SEQ ID NO: 2.

In some embodiments, the engineered RNA ligase is expressed as a fusion protein. In some embodiments, the engineered RNA ligase described herein can be fused to a variety of polypeptide sequences, such as, by way of example and not limitation, polypeptide tags that can be used for detection and/or purification. In some embodiments, the fusion protein of the engineered RNA ligase comprises a glycine-histidine or histidine-tag (His-tag). In some embodiments, the fusion protein of the engineered RNA ligase comprises a polylysine, for example, 2-12 contiguous lysine residues. In some embodiments, the fusion protein of the engineered RNA ligase comprises an epitope tag, such as c-myc, FLAG, V5, or hemagglutinin (HA). In some embodiments, the fusion protein of the engineered RNA ligase comprises a GST, SUMO, Strep, MBP, or GFP tag. In some embodiments, the fusion is to the amino (N-) terminus of engineered RNA ligase polypeptide. In some embodiments, the fusion is to the carboxy (C-) terminus of the engineered RNA ligase polypeptide.

In some embodiments, the present disclosure further provides functional fragments or biologically active fragments of engineered RNA ligase polypeptides described herein. Thus, for each and every embodiment herein of an engineered RNA ligase, a functional fragment or biologically active fragment of the engineered RNA ligase is provided herewith. In some embodiments, a functional fragment or biologically active fragments of an engineered RNA ligase comprises at least about 90%, 91%, 92%, 93%, 94%, 95%, 96%, 97%, 98%, or 99% of the activity of the RNA ligase polypeptide from which it was derived (i.e., the parent RNA ligase). In some embodiments, functional fragments or biologically active fragments comprise at least 90%, 91%, 92%, 93%, 94%, 95%, 96%, 97%, 98%, or 99% of the parent sequence of the RNA ligase. In some embodiments, the functional fragment will be truncated by less than 5, less than 10, less than 15, less than 10, less than 25, less than 30, or less than 35 amino acids.

In some embodiments, the functional fragments or biologically active fragments of the engineered RNA ligase polypeptide described herein include at least a mutation or mutation set in the amino acid sequence of the engineered RNA ligase described herein. Accordingly, in some embodiments, the functional fragments or biologically active fragments of the engineered RNA ligase displays the enhanced or improved property associated with the mutation or mutation set in the parent RNA ligase.

In some embodiments, the engineered RNA ligase is purified, as described herein. In some embodiments, the purified preparation has the engineered RNA ligase at least 60%, 70%, 80%, 85%, 90%, or 95% greater of the protein content of the preparation.

In some embodiments, an engineered RNA ligase described herein is provided immobilized on a substrate or support medium, such as a solid substrate, a porous substrate, a membrane, or particles. The polypeptide can be entrapped in matrixes or membranes. In some embodiments, matrices include polymeric materials such as calcium-alginate, agar, k-carrageenin, polyacrylamide, and collagen. In some embodiments, the solid matrices, includes, among others, activated carbon, porous ceramic, and diatomaceous earth. In some embodiments, the matrix is a particle, a membrane, or a fiber. Types of membranes include, among others, nylon, cellulose, polysulfone, or polyacrylate.

In some embodiments, the engineered RNA ligase is immobilized on the surface of a support material. In some embodiments, the polypeptide is adsorbed on the support material. In some embodiments, the polypeptide is immobilized on the support material by covalent attachment. Support materials include, among others, inorganic materials, such as alumina, silica, porous glass, ceramics, diatomaceous earth, clay, and bentonite, or organic materials, such as cellulose (CMC, DEAE-cellulose), starch, activated carbon, polyacrylamide, polystyrene, and ion-exchange resins, such as Amberlite, Sephadex, and Dowex.

### Polynucleotides Encoding Engineered Polypeptides, Expression Vectors and Host Cells

In another aspect, the present disclosure provides a recombinant polynucleotide comprising a polynucleotide sequence encoding the engineered RNA ligase described herein. In some embodiments, the recombinant polynucleotide is operably linked to one or more control sequences to create a recombinant polynucleotide construct capable of expressing the engineered RNA ligase. In some embodiments, the expression constructs encoding an engineered RNA ligase polypeptide is introduced into appropriate host cells to express the encoded RNA ligase polypeptide.

As will be apparent to the skilled artisan, availability of a protein sequence and the knowledge of the codons corresponding to the various amino acids provide a description of all the polynucleotides capable of encoding the subject RNA ligase polypeptides. The degeneracy of the genetic code, where the same amino acids are encoded by alternative or synonymous codons, allows an extremely large number of nucleic acids to be made, all of which encode an engineered RNA ligase of the present disclosure. Thus, the present disclosure provides methods and compositions for the production of each and every possible variation of polynucleotides that could be made that encode the engineered RNA ligase polypeptides described herein by selecting combinations based on the possible codon choices, and all such variations are to be considered specifically disclosed for any polypeptide described herein, including the amino acid sequences presented in the Examples (e.g., Tables 3, 4, 5, 6, 7.1, 7.2, 8, 9, 10, 11.3, 11.4, and 12) and in the Sequence Listing.

In some embodiments, the codons are preferably optimized for utilization by the chosen host cell for protein production. In some embodiments, preferred codons in bacteria are used for expression in bacteria. In some embodiments, preferred codons in fungal cells are used for expression in fungal cells. In some embodiments, preferred codons in mammalian cells are used for expression in mammalian cells. In some embodiments, codon optimized polynucleotides encoding an engineered RNA ligase polypeptide described herein contain preferred codons at about 40%, 50%, 60%, 70%, 80%, 90%, or greater than 90% of the codon positions in the full length coding region.

Accordingly, by way of example and not limitations, in some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of an even numbered SEQ ID NO. of SEQ ID NOs: 2-220, 224-252, and 270-958, or to a reference sequence corresponding to an even numbered SEQ ID NO. of SEQ ID NOs: 2-220, 224-252, and 270-958, wherein the amino acid sequence comprises one or more substitutions relative to the reference sequence corresponding to residues 12 to 343 of SEQ ID NO: 2, 4, 106, 218, 286, 396, 436, 520, 552, or 582, or relative to the reference sequence corresponding to SEQ ID NO: 2, 4, 106, 218, 286, 396, 436, 520, 552, or 582.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of SEQ ID NO: 2, 4, 106, 218, 286, 396, 436, 520, 552, or 582, or to a reference sequence corresponding to SEQ ID NO: 2, 4, 106, 218, 286, 396, 436, 520, 552, or 582, wherein the amino acid sequence comprises one or more substitutions relative to the reference sequence corresponding to residues 12 to 343 of SEQ ID NO: 2, 4, 106, 218, 286, 396, 436, 520, 552, or 582, or relative to the reference sequence corresponding to SEQ ID NO: 2, 4, 106, 218, 286, 396, 436, 520, 552, or 582.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 343 of SEQ ID NO: 2, or to the reference sequence corresponding to SEQ ID NO: 2, wherein the amino acid sequence comprises one or more substitutions relative to the reference sequence corresponding to residues 12 to 343 of SEQ ID NO: 2, or relative to the reference sequence corresponding to SEQ ID NO: 2.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 343 of SEQ ID NO: 2, or to the reference sequence corresponding to SEQ ID NO: 2, wherein the amino acid sequence comprises one or more substitutions relative to the reference sequence corresponding to residues 12 to 343 of SEQ ID NO: 2, or relative to the reference sequence corresponding to SEQ ID NO: 2.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 343 of SEQ ID NO: 4, 106, 218, 286, 396, 436, 520, 552, or 582, or to the reference sequence corresponding to SEQ ID NO: 4, 106, 218, 286, 396, 436, 520, 552, or 582, wherein the amino acid sequence comprises one or more substitutions relative to the reference sequence corresponding to residues 12 to 343 of SEQ ID NO: 2, or relative to the reference sequence corresponding to SEQ ID NO: 2.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 343 of an even-numbered SEQ ID NO. of SEQ ID NOs: 4-220, 224-252, and 270-958, or to a reference sequence corresponding to an even-numbered SEQ ID NO. of SEQ ID NOs: 4-220, 224-252, and 270-958, wherein the amino acid sequence comprises one or more substitutions relative to the reference sequence corresponding to residues 12 to 343 of SEQ ID NO: 2, or relative to the reference sequence corresponding to SEQ ID NO: 2.

In some embodiments, the recombinant polynucleotide comprises a polynucleotide sequence encoding an engineered RNA ligase comprising an amino acid sequence comprising at least a substitution at amino acid position 11, 12, 14, 16, 17, 23, 25, 26, 30, 32, 33, 38, 40, 52, 57, 60, 68, 69, 71, 73, 75, 79, 82, 83, 92, 93, 95, 96, 98, 100, 101, 102, 103, 111, 114, 117, 118, 119, 120, 135, 136, 141, 142, 145, 147, 156, 167, 168, 170, 171, 174, 177, 179, 183, 184, 185, 188, 189, 190, 191, 192, 193, 196, 200, 202, 205, 206, 207, 209, 221, 229, 254, 255, 259, 270, 271, 287, 288, 289, 292, 295, 296, 300, 307, 320, 327, 332, 333, 334, 335, 336, 337, 339, 342, or 343, or combinations thereof, wherein the amino acid positions are relative to the reference sequence corresponding to residues 12 to 343 of SEQ ID NO: 2, or relative to the reference sequence corresponding to SEQ ID NO: 2.

In some embodiments, the recombinant polynucleotide comprises a polynucleotide sequence encoding an engineered RNA ligase comprising an amino acid sequence comprising at least a substitution at amino acid position 33, 38, 71, 73, 75, 96, 98, 101, 114, 117, 136, 156, 179, 184, 191, 196, 221, 289, 292, 296, 320, 335, 336, or 339, or combinations thereof, wherein the amino acid positions are relative to the reference sequence corresponding to residues 12 to 343 of SEQ ID NO: 2, or relative to the reference sequence corresponding to SEQ ID NO: 2.

In some embodiments, the recombinant polynucleotide comprises a polynucleotide sequence encoding an engineered RNA ligase comprising an amino acid sequence comprising at least a substitution at amino acid position 14, 32, 33, 38, 40, 57, 60, 71, 75, 82, 92, 96, 98, 101, 114, 141, 142, 145, 170, 171, 174, 179, 184, 191, 193, 196, 202, 205, 207, 209, 221, 255, 320, 327, 339, or 342, or combinations thereof, wherein the amino acid positions are relative to the reference sequence corresponding to residues 12 to 343 of SEQ ID NO: 2, or relative to the reference sequence corresponding to SEQ ID NO: 2.

In some embodiments, the recombinant polynucleotide comprises a polynucleotide sequence encoding an engineered RNA ligase comprising an amino acid sequence comprising at least a substitution at an amino acid position set forth in Tables 3, 4, 5, 6, 7.1, 7.2, 8, 9, 10, 11.3, 11.4, and 12, wherein the amino acid positions are relative to the reference sequence corresponding to residues 12 to 343 of SEQ ID NO: 2, or relative to the reference sequence corresponding to SEQ ID NO: 2.

In some embodiments, the recombinant polynucleotide comprises a polynucleotide sequence encoding an engineered RNA ligase comprising an amino acid sequence comprising at least one substitution set forth in Tables 3, 4, 5, 6, 7.1, 7.2, 8, 9, 10, 11.3, 11.4, and 12, wherein the amino acid positions are relative to the reference sequence corresponding to residues 12 to 343 of SEQ ID NO: 2, or relative to the reference sequence corresponding to SEQ ID NO: 2.

In some embodiments, the recombinant polynucleotide comprises a polynucleotide sequence encoding an engineered RNA ligase comprising an amino acid sequence comprising at least a substitution or substitution set at amino acid position(s) set forth in Tables 3, 4, 5, 6, 7.1, 7.2, 8, 9, 10, 11.3, 11.4, and 12, wherein the amino acid positions are relative to the reference sequence corresponding to residues 12 to 343 of SEQ ID NO: 2, or relative to the reference sequence corresponding to SEQ ID NO: 2.

In some embodiments, the recombinant polynucleotide comprises a polynucleotide sequence encoding an engineered RNA ligase comprising an amino acid sequence comprising at least a substitution or substitution set of a RNA ligase variant forth in Tables 3, 4, 5, 6, 7.1, 7.2, 8, 9, 10, 11.3, 11.4, and 12, wherein the amino acid positions are relative to the reference sequence corresponding to residues 12 to 343 of SEQ ID NO: 2, or relative to the reference sequence corresponding to SEQ ID NO: 2.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of SEQ ID NO: 4, 106, 218, 286, 396, 436, 520, 552, or 582, or to a reference sequence corresponding to SEQ ID NO: 4, 106, 218, 286, 396, 436, 520, 552, or 582.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of an even-numbered SEQ ID NO of SEQ ID NOs: 4-220, 224-252, and 270-958, or to a reference sequence corresponding to an even-numbered SEQ ID NO of SEQ ID NOs: 4-220, 224-252, and 270-958.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of SEQ ID NO: 4, 106, 218, 286, 396, 436, 520, 552, or 582, or to a reference sequence corresponding to SEQ ID NO: 4, 106, 218, 286, 396, 436, 520, 552, or 582, wherein the amino acid sequence comprises one or more substitutions relative to the reference sequence corresponding to residues 12 to 343 of SEQ ID NO: 4, 106, 218, 286, 396, 436, 520, 552, or 582, or relative to the reference sequence corresponding to SEQ ID NO: 4, 106, 218, 286, 396, 436, 520, 552, or 582.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of an even-numbered SEQ ID NO of SEQ ID NOs: 4-220, 224-252, and 270-958, or to a reference sequence corresponding to an even-numbered SEQ ID NO of SEQ ID NOs: 4-220, 224-252, and 270-958, wherein the amino acid sequence comprises one or more substitutions relative to the reference sequence corresponding to residues 12 to 343 of SEQ ID NO: 4, 106, 218, 286, 396, 436, 520, 552, or 582, or relative to the reference sequence corresponding to SEQ ID NO: 4, 106, 218, 286, 396, 436, 520, 552, or 582.

In some embodiments, the recombinant polynucleotide comprises a polynucleotide sequence encoding an engineered RNA ligase comprising an amino acid sequence comprising at least a substitution at amino acid position 11, 12, 14, 16, 17, 23, 25, 26, 30, 32, 33, 38, 40, 52, 57, 60, 68, 69, 71, 73, 75, 79, 82, 83, 92, 93, 95, 96, 98, 100, 101, 102, 103, 111, 114, 117, 118, 119, 120, 135, 136, 141, 142, 145, 147, 156, 167, 168, 170, 171, 174, 177, 179, 183, 184, 185, 188, 189, 190, 191, 192, 193, 196, 200, 202, 205, 206, 207, 209, 221, 229, 254, 255, 259, 270, 271, 287, 288, 289, 292, 295, 296, 300, 307, 320, 327, 332, 333, 334, 335, 336, 337, 339, 342, or 343, or combinations thereof, wherein the amino acid positions are relative to the reference sequence corresponding to residues 12 to 343 of SEQ ID NO: 4, 106, 218, 286, 396, 436, 520, 552, or 582, or relative to the reference sequence corresponding to SEQ ID NO: 4, 106, 218, 286, 396, 436, 520, 552, or 582.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of SEQ ID NO: 4, or to a reference sequence corresponding to SEQ ID NO: 4, wherein the amino acid sequence comprises one or more substitutions relative to the reference sequence corresponding to residues 12 to 343 of SEQ ID NO: 4, or relative to the reference sequence corresponding to SEQ ID NO: 4.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of an even-numbered SEQ ID NO. of SEQ ID NOs: 4-104, or to a reference sequence corresponding to an even-numbered SEQ ID NO. of SEQ ID NOs: 4-104, wherein the amino acid sequence comprises one or more substitutions relative to the reference sequence corresponding to residues 12 to 343 of SEQ ID NO: 4, or relative to the reference sequence corresponding to SEQ ID NO: 4.

In some embodiments, the recombinant polynucleotide comprises a polynucleotide sequence encoding an engineered RNA ligase comprising an amino acid sequence comprising at least a substitution set at amino acid position(s) 33/101/221/320/335/336/339, 71/92/171/184/202, 40/92/171/202/327, 196/300/335/339, 33/184/221/300/320, 335/336/339, 184/196/221/336, 33/184/320/335/336, 207/209/221/300/320/336/339, 101/196/221/300/335, 207/209/300/320, 40/170/171/202/327, 33/101/184/196/209/336/339, 40/71/92/171/184/327, 207/335/336/339, 33/101/196/300/320/335/336/339, 57/171/179/184/202/327, 57/92/202/327, 33/101/207/221/300/335/336/339, 33/221/336, 196/221/300/335/336/339, 33/184/221/336/339, 33/184/196/221/300, 101/196/207/209/221/300/336/339, 101/184/335/336/339, 33/184/196/320/336/339, 33/184/196/300/320/335/336/339, 184/320/335/336/339, 184/196/207/209/300/335/336/339, 184/196/320/335/336/339, 33/196/209/221/339, 184/320/335/336/339, 101/209/335/336/339, 101/196/209/339, 40/71/82/170/171/179/202/327, 320/335/336/339, 101/184/300/335/336/339, 33/101/196/209/335/336/339, 33/196/300/320, 221/335, 33/184/196/207/221, 221/335/336/339, 196/209/300/335/336/339, 184/196/300/320, 57/171/202/327, 40/71/171/184, 184/196/221, 33/196/207/335/336, 32/57/71/202/327/342, 33/196/221/336, 101/221/300/320/335/336/339, 33/101/207/221, 101/184/196/209/221, 33/207/209/300/335/336/339, or 33/83/184/196/300/336, wherein the amino acid positions are relative to the reference sequence corresponding to residues 12 to 343 of SEQ ID NO: 4, or relative to the reference sequence corresponding to SEQ ID NO: 4.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of SEQ ID NO: 106, or to a reference sequence corresponding to SEQ ID NO: 106, wherein the amino acid sequence comprises one or more substitutions relative to the reference sequence corresponding to residues 12 to 343 of SEQ ID NO: 106, or relative to the reference sequence corresponding to SEQ ID NO: 106.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of an even-numbered SEQ ID NO of SEQ ID NOs: 218-220 and 224-252, or to a reference sequence corresponding to an even-numbered SEQ ID NO of SEQ ID NOs: 218-220 and 224-252, wherein the amino acid sequence comprises one or more substitutions relative to the reference sequence corresponding to residues 12 to 343 of SEQ ID NO: 106, or relative to the reference sequence corresponding to SEQ ID NO: 106.

In some embodiments, the recombinant polynucleotide comprises a polynucleotide sequence encoding an engineered RNA ligase comprising an amino acid sequence comprising at least a substitution or substitution set at amino acid position(s) 38/71/114/184/191/196, 141/170/171/174, 114, 57/60/170/171/174/192/196/320, 60/92/170/171, 141/174/196, 57/170/171/174, 141/184, 57/60/141/170/171/174/192, 60/170/174, 141/170/171, 69/71/114/184, 38/40, 40/71/98/184/259, 32/60/141/170/171/174/196/320, 32/170/207/320, or 196, wherein the amino acid positions are relative to the reference sequence corresponding to residues 12 to 343 of SEQ ID NO: 106, or relative to the reference sequence corresponding to SEQ ID NO: 106.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of SEQ ID NO: 218, or to a reference sequence corresponding to SEQ ID NO: 218, wherein the amino acid sequence comprises one or more substitutions relative to the reference sequence corresponding to residues 12 to 343 of SEQ ID NO: 218, or relative to the reference sequence corresponding to SEQ ID NO: 218.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of an even-numbered SEQ ID NO of SEQ ID NOs: 270-312, or to a reference sequence corresponding to an even-numbered SEQ ID NO of SEQ ID NOs: 270-312, wherein the amino acid sequence comprises one or more substitutions relative to the reference sequence corresponding to residues 12 to 343 of SEQ ID NO: 218, or relative to the reference sequence corresponding to SEQ ID NO: 218.

In some embodiments, the recombinant polynucleotide comprises a polynucleotide sequence encoding an engineered RNA ligase comprising an amino acid sequence comprising at least a substitution or substitution set at amino acid position(s) 102, 17, 190, 16, 68, 23, 270, 117/229, or 117, wherein the amino acid positions are relative to the reference sequence corresponding to residues 12 to 343 of SEQ ID NO: 218, or relative to the reference sequence corresponding to SEQ ID NO: 218.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of SEQ ID NO: 286, or to a reference sequence corresponding to SEQ ID NO: 286, wherein the amino acid sequence comprises one or more substitutions relative to the reference sequence corresponding to residues 12 to 343 of SEQ ID NO: 286, or relative to the reference sequence corresponding to SEQ ID NO: 286.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of an even-numbered SEQ ID NO of SEQ ID NOs: 314-426, or to a reference sequence corresponding to an even-numbered SEQ ID NO of SEQ ID NOs: 314-426, wherein the amino acid sequence comprises one or more substitutions relative to the reference sequence corresponding to residues 12 to 343 of SEQ ID NO: 286, or relative to the reference sequence corresponding to SEQ ID NO: 286.

In some embodiments, the recombinant polynucleotide comprises a polynucleotide sequence encoding an engineered RNA ligase comprising an amino acid sequence comprising at least a substitution or substitution set at amino acid position(s) 296, 114/117, 26, 30, 117/118, 117/119, 14, 183, 117, 185, 307, 12, 93/135, 135, 92, 288, 117/120, 79, 100, 287, 292, 25, 75, 327, or 111, wherein the amino acid positions are relative to the reference sequence corresponding to residues 12 to 343 of SEQ ID NO: 286, or relative to the reference sequence corresponding to SEQ ID NO: 286.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of SEQ ID NO: 396, or to a reference sequence corresponding to SEQ ID NO: 396, wherein the amino acid sequence comprises one or more substitutions relative to the reference sequence corresponding to residues 12 to 343 of SEQ ID NO: 396, or relative to the reference sequence corresponding to SEQ ID NO: 396.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of an even-numbered SEQ ID NO of SEQ ID NOs: 428-488, or to a reference sequence corresponding to an even-numbered SEQ ID NO of SEQ ID NOs: 428-488, wherein the amino acid sequence comprises one or more substitutions relative to the reference sequence corresponding to residues 12 to 343 of SEQ ID NO: 396, or relative to the reference sequence corresponding to SEQ ID NO: 396.

In some embodiments, the recombinant polynucleotide comprises a polynucleotide sequence encoding an engineered RNA ligase comprising an amino acid sequence comprising at least a substitution or substitution set at amino acid position(s) 102/119, 75/102, 75/102/117, 75/119, 75/136/296, 75/327, 75/117/119, 119, 119/296, 14/102/119, 14/102/296, 14/75, 14/75/119, 14/75/119/327, 14/75/117/119, 14/75/117/119/296, 14/119, 14/296, 14/30/270, 14/25, 14/117, 14/117/119, 14/117/119/296, 14/117/296/327, 26/75/327, 25/75, 25/30/102, 117, 117/119, or 117/119/296, wherein the amino acid positions are relative to the reference sequence corresponding to residues 12 to 343 of SEQ ID NO: 396, or relative to the reference sequence corresponding to SEQ ID NO: 396.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of SEQ ID NO: 436, or to a reference sequence corresponding to SEQ ID NO: 436, wherein the amino acid sequence comprises one or more substitutions relative to the reference sequence corresponding to residues 12 to 343 of SEQ ID NO: 436, or relative to the reference sequence corresponding to SEQ ID NO: 436.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of an even-numbered SEQ ID NO of SEQ ID NOs: 490-534, or to a reference sequence corresponding to an even-numbered SEQ ID NO of SEQ ID NOs: 490-534, wherein the amino acid sequence comprises one or more substitutions relative to the reference sequence corresponding to residues 12 to 343 of SEQ ID NO: 436, or relative to the reference sequence corresponding to SEQ ID NO: 436.

In some embodiments, the recombinant polynucleotide comprises a polynucleotide sequence encoding an engineered RNA ligase comprising an amino acid sequence comprising at least a substitution at amino acid position 332, 156, 177, 333, 96, 11, 189, 327, 25, 73, 98, 335, 95, 188, or 52, or combinations thereof, wherein the amino acid positions are relative to the reference sequence corresponding to residues 12 to 343 of SEQ ID NO: 436, or relative to the reference sequence corresponding to SEQ ID NO: 436.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of SEQ ID NO: 520, or to a reference sequence corresponding to SEQ ID NO: 520, wherein the amino acid sequence comprises one or more substitutions relative to the reference sequence corresponding to residues 12 to 343 of SEQ ID NO: 520, or relative to the reference sequence corresponding to SEQ ID NO: 520.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of an even-numbered SEQ ID NO of SEQ ID NOs: 536-554, or to a reference sequence corresponding to an even-numbered SEQ ID NO of SEQ ID NOs: 536-554, wherein the amino acid sequence comprises one or more substitutions relative to the reference sequence corresponding to residues 12 to 343 of SEQ ID NO: 520, or relative to the reference sequence corresponding to SEQ ID NO: 520.

In some embodiments, the recombinant polynucleotide comprises a polynucleotide sequence encoding an engineered RNA ligase comprising an amino acid sequence comprising at least a substitution set at amino acid positions 96/98/156/189/335, 73/96/98/189/335, 73/177/189, 73/98/177/189/335, 25/73/95/96/98/189/327, 25/98/189, 73/95/96/156/177, 73/335, 73/96/98/156/335, or 73/177/189/333, wherein the amino acid positions are relative to the reference sequence corresponding to residues 12 to 343 of SEQ ID NO: 520, or relative to the reference sequence corresponding to SEQ ID NO: 520.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of SEQ ID NO: 552, or to a reference sequence corresponding to SEQ ID NO: 552, wherein the amino acid sequence comprises one or more substitutions relative to the reference sequence corresponding to residues 12 to 343 of SEQ ID NO: 552, or relative to the reference sequence corresponding to SEQ ID NO: 552.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of an even-numbered SEQ ID NO of SEQ ID NOs: 556-584, or to a reference sequence corresponding to an even-numbered SEQ ID NO of SEQ ID NOs: 556-584, wherein the amino acid sequence comprises one or more substitutions relative to the reference sequence corresponding to residues 12 to 343 of SEQ ID NO: 552, or relative to the reference sequence corresponding to SEQ ID NO: 552.

In some embodiments, the recombinant polynucleotide comprises a polynucleotide sequence encoding an engineered RNA ligase comprising an amino acid sequence comprising at least a substitution at amino acid position 292, 147, 255, 295, 75, 300, 333, 200, 289, 95, or 168, or combinations thereof, wherein the amino acid positions are relative to the reference sequence corresponding to residues 12 to 343 of SEQ ID NO: 552, or relative to the reference sequence corresponding to SEQ ID NO: 552.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of SEQ ID NO: 582, or to a reference sequence corresponding to SEQ ID NO: 582, wherein the amino acid sequence comprises one or more substitutions relative to the reference sequence corresponding to residues 12 to 343 of SEQ ID NO: 582, or relative to the reference sequence corresponding to SEQ ID NO: 582.

In some embodiments, the recombinant polynucleotide comprises a polynucleotide sequence encoding an engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of an even-numbered SEQ ID NO of SEQ ID NOs: 586-958, or to a reference sequence corresponding to an even-numbered SEQ ID NO of SEQ ID NOs: 586-958, wherein the amino acid sequence comprises one or more substitutions relative to the reference sequence corresponding to residues 12 to 343 of SEQ ID NO: 582, or relative to the reference sequence corresponding to SEQ ID NO: 582.

In some embodiments, the recombinant polynucleotide comprises a polynucleotide sequence encoding an engineered RNA ligase comprising an amino acid sequence comprising at least a substitution or substitution set at amino acid position(s) 95/156/337, 189/200/271, 200/271/289/337, 200/271/289, 189/200, 156/200/289, 156/168/189/271/300, 156/189/200/289, 156/168/289, 95/156/271/289, 156/333, 95/200/289/337, 168/200/289, 189/200/289, 333/337, 103/156/271, 95/156/200/300/333, 156/271/289/333, 95/156/271, 95/189/289, 95/156/168/189/271/289/300, 189/271/289, 95/168/271/300, 156/189/289/337, 200/271, 156/189/333, 189/289, 156/189/200/300/333/337, 156/189/200/271/289/337, 156/200/271/289/333/337, 168/271/300, 289/337, 156/168/337, 156/168/189/289, 156/271/289, 189/300, 156/289/300/337, 168/189/289, 95/156/168/300, 156/200/300/337, 289/333/337, 95/189/200/300/337, 95/156/189/200/271/333, 200/271/289/300, 289/333, 189/271/300, 95/300, 95/189/333/337, 95/189/271/300, 95/189/200/289, 168, 156/200/271/289/337, 95/168/289, 156/289, 95/156/189/271/289, 156/168/189/200/289/333, 156/300/333/337, 95/156/189/200/271/337, 271, 156/271, 156/271/300/333, 95/168/200, 168/271, 95/200/333, 189/200/271/289/333, 95/168/189/333/337, 189, 189/333/337, 156/189/271/289/333, 289, 95/156/200/271/289, 289/300/333, 95/189/289/337, 95/156/168/189/289, 271/289, 333, 168/337, 156/189/271/289, 95/156/189/337, 156/189/271/300, 156/168/289/333, 168/189/289/333, 95/333, 333/343, 156/189/289, 95/289/300, 95/156/189/200/300/333/337, 189/200/271/289/337, 95/156/168/189/289/337, 95/289, 156/289/333/337, 156/289/337, 337, 168/289, 95/156/300, 95/156/289, 95/189/271/333/337, 95/168/300, 95/156/168/271/333/337, 95/189/289/333/337, 156/168/200/289/334, 95/156/168/189/271/289, 156/189/200/289/337, 200/289, 200/289/333/337, 95/168/189/200/289, 168/200, 156/168/271/289, 189/206/289, 156/200/289/333/335, 156/200/289/333, 189/200/289/333, 189/289/335, 189/254/289/333, 167/189/206/289/333/335, 156/200/333, 156/200/289/335, 156/189/289/335, 335, 156/167/189/289, 156/189/200/289/335, 200/289/333/335, 156/189/200/289/333, 254/333/335, 189/254/333/335, 189/335, 156/189/254/289, 156/289/333, 289/335, 189/200/254/289/333, 156/189/289/333, 25/156/189/289, 156/189/206/289/333/335, 289/333/335, 156/254/289/333, 156/289/333/335, 254/289, 200/289/333, 156/189/200, 156/254/333/335, 156, 254/289/335, 156/206/289, 156/289/335, 206/289/333/335, 156/189/254/289/333, 156/254/289, 200/289/335, 156/189/206/289, 156/189/200/206/254/289/333, 25/254/333, 189/289/333/335, 156/189/200/289/333/335, 254/289/333, or 156/200/333/335, wherein the amino acid positions are relative to the reference sequence corresponding to residues 12 to 343 of SEQ ID NO: 582, or relative to the reference sequence corresponding to SEQ ID NO: 582.

In some embodiments, the recombinant polynucleotide comprises a polynucleotide sequence encoding an engineered RNA ligase comprising an amino acid sequence comprising at least a substitution at an amino acid position set forth in Tables 3, 4, 5, 6, 7.1, 7.2, 8, 9, 10, 11.3, 11.4, and 12, wherein the amino acid positions are relative to the reference sequence corresponding to residues 12 to 343 of SEQ ID NO: 2, 4, 106, 218, 286, 396, 436, 520, 552, or 582, or relative to the reference sequence corresponding to SEQ ID NO: 2, 4, 106, 218, 286, 396, 436, 520, 552, or 582.

In some embodiments, the recombinant polynucleotide comprises a polynucleotide sequence encoding an engineered RNA ligase comprising an amino acid sequence comprising at least one substitution set forth in Tables 3, 4, 5, 6, 7.1, 7.2, 8, 9, 10, 11.3, 11.4, and 12, wherein the amino acid positions are relative to the reference sequence corresponding to residues 12 to 343 of SEQ ID NO: 2, 4, 106, 218, 286, 396, 436, 520, 552, or 582, or relative to the reference sequence corresponding to SEQ ID NO: 2, 4, 106, 218, 286, 396, 436, 520, 552, or 582.

In some embodiments, the recombinant polynucleotide comprises a polynucleotide sequence encoding an engineered RNA ligase comprising an amino acid sequence comprising at least a substitution or substitution set at amino acid position(s) set forth in 3, 4, 5, 6, 7.1, 7.2, 8, 9, 10, 11.3, 11.4, and 12, wherein the amino acid positions are relative to the reference sequence corresponding to residues 12 to 343 of SEQ ID NO: 2, 4, 106, 218, 286, 396, 436, 520, 552, or 582, or relative to the reference sequence corresponding to SEQ ID NO: 2, 4, 106, 218, 286, 396, 436, 520, 552, or 582.

In some embodiments, the recombinant polynucleotide comprises a polynucleotide sequence encoding an engineered RNA ligase comprising an amino acid sequence comprising at least a substitution or substitution set of an RNA ligase variant set forth in Tables 3, 4, 5, 6, 7.1, 7.2, 8, 9, 10, 11.3, 11.4, and 12, wherein the amino acid positions are relative to the reference sequence corresponding to residues 12 to 343 of SEQ ID NO: 2, 4, 106, 218, 286, 396, 436, 520, 552, or 582, or relative to the reference sequence corresponding to SEQ ID NO: 2, 4, 106, 218, 286, 396, 436, 520, 552, or 582.

In some embodiments, the recombinant polynucleotide comprises a polynucleotide sequence encoding an engineered RNA ligase comprising an amino acid sequence at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence comprising a substitution or substitution set of an RNA ligase variant set forth in Tables 3, 4, 5, 6, 7.1, 7.2, 8, 9, 10, 11.3, 11.4, and 12, wherein the amino acid positions are relative to the reference sequence corresponding to residues 12 to 343 of SEQ ID NO: 2, 4, 106, 218, 286, 396, 436, 520, 552, or 582, or relative to the reference sequence corresponding to SEQ ID NO: 2, 4, 106, 218, 286, 396, 436, 520, 552, or 582.

In some embodiments, the recombinant polynucleotide comprises a polynucleotide sequence encoding an engineered RNA ligase comprising an amino acid sequence comprising residues 12 to 343 of an even numbered SEQ ID NO. of SEQ ID NOs: 4-220, 224-252, and 270-958, or an amino acid sequence comprising an even numbered SEQ ID NO. of SEQ ID NOs: 4-220, 224-252, and 270-958.

In some embodiments, the recombinant polynucleotide comprises a polynucleotide sequence encoding an engineered RNA ligase comprising an amino acid sequence comprising residues 12 to 343 of SEQ ID NO: 2, 4, 106, 218, 286, 396, 436, 520, 552, or 582, or an amino acid sequence comprising SEQ ID NO: 2, 4, 106, 218, 286, 396, 436, 520, 552, or 582.

In some embodiments, the recombinant polynucleotide comprises a polynucleotide sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference polynucleotide sequence corresponding to nucleotide residues 34 to 1029 of an odd-numbered SEQ ID NO. of SEQ ID NO: 3, 105, 217, 285, 395, 435, 519, 551, or 581, or to a reference polynucleotide sequence corresponding to SEQ ID NO: 3, 105, 217, 285, 395, 435, 519, 551, or 581, wherein the recombinant polynucleotide encodes an RNA ligase.

In some embodiments, the recombinant polynucleotide comprises a polynucleotide sequence having at least 70%, 75%, 80%, 81%, 82%, 83%, 84%, 85%, 90%, 91%, 92%, 93%, 94%, 95%, 96%, 97%, 98%, 99%, or more sequence identity to a reference polynucleotide sequence corresponding to nucleotide residues 34 to 1029 of an odd numbered SEQ ID NO. of SEQ ID NOs: 3-219, 223-251, and 269-957, or to a reference polynucleotide sequence corresponding an odd numbered SEQ ID NO. of SEQ ID NOs: 3-219, 223-251, and 269-957, wherein the recombinant polynucleotide encodes an RNA ligase.

In some embodiments, the recombinant polynucleotide comprises a polynucleotide sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to nucleotide residues 34 to 1029 of SEQ ID NO: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 725, 727, 729, 731, 733, 735, 737, 739, 741, 743, 745, 747, 749, 751, 753, 755, 757, 759, 761, 763, 765, 767, 769, 771, 773, 775, 777, 779, 781, 783, 785, 787, 789, 791, 793, 795, 797, 799, 801, 803, 805, 807, 809, 811, 813, 815, 817, 819, 821, 823, 825, 827, 829, 831, 833, 835, 837, 839, 841, 843, 845, 847, 849, 851, 853, 855, 857, 859, 861, 863, 865, 867, 869, 871, 873, 875, 877, 879, 881, 883, 885, 887, 889, 891, 893, 895, 897, 899, 901, 903, 905, 907, 909, 911, 913, 915, 917, 919, 921, 923, 925, 927, 929, 931, 933, 935, 937, 939, 941, 943, 945, 947, 949, 951, 953, 955, or 957, wherein the recombinant polynucleotide encodes an RNA ligase.

In some embodiments, the recombinant polynucleotide comprises a polynucleotide sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to the reference sequence corresponding to SEQ ID NO: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 725, 727, 729, 731, 733, 735, 737, 739, 741, 743, 745, 747, 749, 751, 753, 755, 757, 759, 761, 763, 765, 767, 769, 771, 773, 775, 777, 779, 781, 783, 785, 787, 789, 791, 793, 795, 797, 799, 801, 803, 805, 807, 809, 811, 813, 815, 817, 819, 821, 823, 825, 827, 829, 831, 833, 835, 837, 839, 841, 843, 845, 847, 849, 851, 853, 855, 857, 859, 861, 863, 865, 867, 869, 871, 873, 875, 877, 879, 881, 883, 885, 887, 889, 891, 893, 895, 897, 899, 901, 903, 905, 907, 909, 911, 913, 915, 917, 919, 921, 923, 925, 927, 929, 931, 933, 935, 937, 939, 941, 943, 945, 947, 949, 951, 953, 955, or 957, wherein the recombinant polynucleotide encodes an RNA ligase.

In some embodiments, the recombinant polynucleotide comprises a polynucleotide sequence comprising nucleotide residues 34 to 1029 of SEQ ID NO. 3, 105, 217, 285, 395, 435, 519, 551, or 581, or a polynucleotide sequence comprising SEQ ID NOs: 3, 105, 217, 285, 395, 435, 519, 551, or 581.

In some embodiments, the recombinant polynucleotide comprises a polynucleotide sequence comprising nucleotide residues 34 to 1029 of an odd numbered SEQ ID NO. of SEQ ID NOs: 3-219, 223-251, and 269-957, or a polynucleotide sequence comprising an odd numbered SEQ ID NO. of SEQ ID NOs: 3-219, 223-251, and 269-957.

In some embodiments, the recombinant polynucleotide comprises a polynucleotide sequence comprising nucleotide residues 34 to 1029 of SEQ ID NO: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 725, 727, 729, 731, 733, 735, 737, 739, 741, 743, 745, 747, 749, 751, 753, 755, 757, 759, 761, 763, 765, 767, 769, 771, 773, 775, 777, 779, 781, 783, 785, 787, 789, 791, 793, 795, 797, 799, 801, 803, 805, 807, 809, 811, 813, 815, 817, 819, 821, 823, 825, 827, 829, 831, 833, 835, 837, 839, 841, 843, 845, 847, 849, 851, 853, 855, 857, 859, 861, 863, 865, 867, 869, 871, 873, 875, 877, 879, 881, 883, 885, 887, 889, 891, 893, 895, 897, 899, 901, 903, 905, 907, 909, 911, 913, 915, 917, 919, 921, 923, 925, 927, 929, 931, 933, 935, 937, 939, 941, 943, 945, 947, 949, 951, 953, 955, or 957.

In some embodiments, the recombinant polynucleotide comprises a polynucleotide sequence comprising SEQ ID NO: 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163, 165, 167, 169, 171, 173, 175, 177, 179, 181, 183, 185, 187, 189, 191, 193, 195, 197, 199, 201, 203, 205, 207, 209, 211, 213, 215, 217, 219, 221, 223, 225, 227, 229, 231, 233, 235, 237, 239, 241, 243, 245, 247, 249, 251, 269, 271, 273, 275, 277, 279, 281, 283, 285, 287, 289, 291, 293, 295, 297, 299, 301, 303, 305, 307, 309, 311, 313, 315, 317, 319, 321, 323, 325, 327, 329, 331, 333, 335, 337, 339, 341, 343, 345, 347, 349, 351, 353, 355, 357, 359, 361, 363, 365, 367, 369, 371, 373, 375, 377, 379, 381, 383, 385, 387, 389, 391, 393, 395, 397, 399, 401, 403, 405, 407, 409, 411, 413, 415, 417, 419, 421, 423, 425, 427, 429, 431, 433, 435, 437, 439, 441, 443, 445, 447, 449, 451, 453, 455, 457, 459, 461, 463, 465, 467, 469, 471, 473, 475, 477, 479, 481, 483, 485, 487, 489, 491, 493, 495, 497, 499, 501, 503, 505, 507, 509, 511, 513, 515, 517, 519, 521, 523, 525, 527, 529, 531, 533, 535, 537, 539, 541, 543, 545, 547, 549, 551, 553, 555, 557, 559, 561, 563, 565, 567, 569, 571, 573, 575, 577, 579, 581, 583, 585, 587, 589, 591, 593, 595, 597, 599, 601, 603, 605, 607, 609, 611, 613, 615, 617, 619, 621, 623, 625, 627, 629, 631, 633, 635, 637, 639, 641, 643, 645, 647, 649, 651, 653, 655, 657, 659, 661, 663, 665, 667, 669, 671, 673, 675, 677, 679, 681, 683, 685, 687, 689, 691, 693, 695, 697, 699, 701, 703, 705, 707, 709, 711, 713, 715, 717, 719, 721, 723, 725, 727, 729, 731, 733, 735, 737, 739, 741, 743, 745, 747, 749, 751, 753, 755, 757, 759, 761, 763, 765, 767, 769, 771, 773, 775, 777, 779, 781, 783, 785, 787, 789, 791, 793, 795, 797, 799, 801, 803, 805, 807, 809, 811, 813, 815, 817, 819, 821, 823, 825, 827, 829, 831, 833, 835, 837, 839, 841, 843, 845, 847, 849, 851, 853, 855, 857, 859, 861, 863, 865, 867, 869, 871, 873, 875, 877, 879, 881, 883, 885, 887, 889, 891, 893, 895, 897, 899, 901, 903, 905, 907, 909, 911, 913, 915, 917, 919, 921, 923, 925, 927, 929, 931, 933, 935, 937, 939, 941, 943, 945, 947, 949, 951, 953, 955, or 957.

In some embodiments, the present disclosure provides a recombinant polynucleotide capable of hybridizing under highly stringent conditions to a reference polynucleotide encoding an engineered RNA ligase polypeptide described herein, e.g., a recombinant polynucleotide provided in Tables 3, 4, 5, 6, 7.1, 7.2, 8, 9, 10, 11.3, 11.4, and 12, or a reverse complement thereof. In some embodiments, the recombinant polynucleotide hybridizes under highly stringent conditions to a reference polynucleotide corresponding to nucleotide residues 34 to 1029 of SEQ ID NO: 3, 105, 217, 285, 395, 435, 519, 551, or 581, or to a reference polynucleotide sequence corresponding to SEQ ID NO: 3, 105, 217, 285, 395, 435, 519, 551, or 581, or a reverse complement thereof. In some embodiments, the recombinant polynucleotide hybridizes under highly stringent conditions to a reference polynucleotide corresponding to nucleotide residues 34 to 1029 of an odd numbered SEQ ID NO. of SEQ ID NOs: 3-219, 223-251, and 269-957, or a polynucleotide sequence comprising an odd numbered SEQ ID NO. of SEQ ID NOs: 3-219, 223-251, and 269-957, or a reverse complement thereof.

In some embodiments, the present disclosure provides a recombinant polynucleotide capable of hybridizing under highly stringent conditions to a reverse complement of a reference polynucleotide encoding an engineered RNA ligase polypeptide described herein, wherein the recombinant polynucleotide hybridizing under stringent conditions encodes an RNA ligase polypeptide comprising an amino acid sequence having one or more amino acid differences as compared to SEQ ID NO: 2, 4, 106, 218, 286, 396, 436, 520, 552, or 582, at residue positions selected from any positions as set forth in Tables 3, 4, 5, 6, 7.1, 7.2, 8, 9, 10, 11.3, 11.4, and 12. In some embodiments, the recombinant polynucleotide that hybridizes under highly stringent conditions to a reverse complement of a reference polynucleotide encoding an engineered RNA ligase polypeptide described herein encodes an RNA ligase polypeptide having one or more amino acid differences present in an engineered RNA ligase having an amino acid sequence corresponding to residues 12 to 343 of an even numbered SEQ ID NO. of SEQ ID NOs: 4-220, 224-252, and 270-958, or an amino acid sequence comprising an even numbered SEQ ID NO. of SEQ ID NOs: 4-220, 224-252, and 270-958, wherein the amino acid differences are relative to SEQ ID NO: 2, 4, 106, 218, 286, 396, 436, 520, 552, or 582.

In some embodiments, the recombinant polynucleotide that hybridizes under highly stringent conditions comprises a polynucleotide sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference polynucleotide sequence corresponding to nucleotide residues 34 to 1029 of SEQ ID NO: 3, 105, 217, 285, 395, 435, 519, 551, or 581, or to a reference polynucleotide sequence corresponding to SEQ ID NO: 3, 105, 217, 285, 395, 435, 519, 551, or 581, or a reverse complement thereof. In some embodiments, the recombinant polynucleotide that hybridizes under highly stringent conditions comprises a polynucleotide sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference polynucleotide sequence corresponding to nucleotide residues 34-1029 of an odd-numbered SEQ ID NO. of SEQ ID NOs: 3-219, 223-251, and 269-957, or an odd-numbered SEQ ID NO. of SEQ ID NOs: 3-219, 223-251, and 269-957, or a reverse complement thereof.

In some additional embodiments, the polynucleotide hybridizing under highly stringent conditions comprises a polynucleotide sequence having at least 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reverse complement of a polynucleotide reference sequence corresponding to nucleotide residues 34 to 1029 of SEQ ID NO: 3, 105, 217, 285, 395, 435, 519, 551, or 581, or to a reference polynucleotide sequence corresponding to SEQ ID NO: 3, 105, 217, 285, 395, 435, 519, 551, or 581 encodes an engineered RNA ligase polypeptide. In some additional embodiments, the polynucleotide hybridizing under highly stringent conditions comprises a polynucleotide sequence having at least 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reverse complement of a polynucleotide reference sequence corresponding to nucleotide residues 34-1029 of an odd-numbered SEQ ID NO. of SEQ ID NOs: 3-219, 223-251, and 269-957, or an odd-numbered SEQ ID NO. of SEQ ID NOs: 3-219, 223-251, and 269-957 encodes an engineered RNA ligase polypeptide.

In some embodiments, a recombinant polynucleotide encoding any of the RNA ligase provided herein is manipulated in a variety of ways to provide for expression of the polypeptide. In some embodiments, the recombinant polynucleotide encoding the polypeptides are provided as expression vectors where one or more control sequences is operably linked to the recombinant polynucleotide to regulate the expression of the polynucleotide and/or encoded polypeptide. In some embodiments, the control sequences include, among others, promoter sequences, Kozak sequence, leader sequences, polyadenylation sequences, pro-peptide sequences, signal peptide sequences, regulatory elements, and transcription terminators. The techniques for modifying polynucleotides and nucleic acid sequences utilizing recombinant DNA methods are known in the art.

In some embodiments, suitable promoters can be selected based on the host cells used. For bacterial host cells, suitable promoters for directing transcription of the nucleic acid constructs of the present disclosure, include, but are not limited to promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, and prokaryotic beta-lactamase gene (See e.g., Villa-Kamaroff et al., Proc. Natl Acad. Sci. USA, 1978, 75:3727-3731), as well as the tac promoter (See e.g., DeBoer et al., Proc. Natl Acad. Sci. USA, 1983, 80:21-25). Exemplary promoters for fungal host cells, include, but are not limited to promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, and Fusarium oxysporum trypsin-like protease (See e.g., WO 96/00787), as well as the NA2-tpi promoter (a hybrid of the promoters from the genes for Aspergillus niger neutral alpha-amylase and Aspergillus oryzae triose phosphate isomerase), and mutant, truncated, and hybrid promoters thereof. Exemplary yeast cell promoters can be from the genes can be from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH2/GAP), and Saccharomyces cerevisiae 3-phosphoglycerate kinase. Other useful promoters for yeast host cells are known in the art (See e.g., Romanos et al., Yeast, 1992, 8:423-488). Exemplary promoters for use in insect cells include, but are not limited to, polyhedrin, p10, ELT, OpIE2, and hr5/ie1 promoters. Exemplary promoters for use in mammalian cells include, but are not limited to, those from cytomegalovirus (CMV), chicken β-actin promoter fused with the CMV enhancer, Simian vacuolating virus 40 (SV40), from Homo sapiens phosphoglycerate kinase, beta actin, elongation factor-la or glyceraldehyde-3-phosphate dehydrogenase, or from Gallus β-actin.

In some embodiments, the control sequence is also a suitable transcription terminator sequence (i.e., a sequence recognized by a host cell to terminate transcription). In some embodiments, the terminator sequence is operably linked to the 3′ terminus of the nucleic acid sequence encoding the leucine decarboxylase polypeptide. Any suitable terminator which is functional in the host cell of choice finds use in the present invention. For bacterial expression, the transcription terminators can be a Rho-dependent terminators that rely on a Rho transcription factor, or a Rho-independent, or intrinsic terminators, which do not require a transcription factor. Exemplary bacterial transcription terminators are described in Peters et al., J Mol Biol., 2011, 412 (5): 793-813. Exemplary transcription terminators for filamentous fungal host cells can be obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease. Exemplary terminators for yeast host cells can be obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are known in the art (See e.g., Romanos et al., supra). Exemplary terminators for mammalian cells include, but are not limited to those from cytomegalovirus (CMV), Simian virus 40 (SV40), from Homo sapiens growth hormone hGH, from bovine growth hormone BGH, and from human or rabbit beta globulin.

In some embodiments, the control sequence is also a suitable leader sequence (i.e., a non-translated region of an mRNA that is important for translation by the host cell). In some embodiments, the leader sequence is operably linked to the 5′ terminus of the nucleic acid sequence encoding the RNA ligase polypeptide. Any suitable leader sequence that is functional in the host cell of choice find use in the present invention. Exemplary leaders for filamentous fungal host cells are obtained from the genes for Aspergillus oryzae TAKA amylase, and Aspergillus nidulans triose phosphate isomerase. Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP). Suitable leaders for mammalian host cells include but are not limited to the 5′-UTR element present in orthopoxvirus mRNA.

In some embodiments, the control sequence comprises a 3′ untranslated nucleic acid region and polyadenylation tail nucleic acid sequence, sequences operably linked to the 3′ terminus of the protein coding nucleic acid sequence which mediate binding to proteins involved in mRNA trafficking and translation and mRNA half-life. Any polyadenylation sequence and 3′ UTR which is functional in the host cell of choice may be used in the present invention. Exemplary polyadenylation sequences for filamentous fungal host cells include, but are not limited to those from the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase. Useful polyadenylation sequences for yeast host cells are also known in the art (See e.g., Guo and Sherman, Mol. Cell. Biol., 1995, 15:5983-5990). Useful polyadenylation and 3′ UTR sequences for mammalian host cells include, but are not limited to, the 3′-UTRs of α- and β-globin mRNAs that harbor several sequence elements that increase the stability and translation of mRNA.

In some embodiments, the control sequence is also a signal peptide (i.e., a coding region that codes for an amino acid sequence linked to the amino terminus of a polypeptide and directs the encoded polypeptide into the cell's secretory pathway). In some embodiments, the 5′ end of the coding sequence of the nucleic acid sequence inherently contains a signal peptide coding region naturally linked in translation reading frame with the segment of the coding region that encodes the secreted polypeptide. Alternatively, in some embodiments, the 5′ end of the coding sequence contains a signal peptide coding region that is foreign to the coding sequence. Any suitable signal peptide coding region which directs the expressed polypeptide into the secretory pathway of a host cell of choice finds use for expression of the engineered polypeptide(s). Effective signal peptide coding regions for bacterial host cells are the signal peptide coding regions include, but are not limited to those obtained from the genes for Bacillus NCIB 11837 maltogenic amylase, Bacillus stearothermophilus alpha-amylase, Bacillus licheniformis subtilisin, Bacillus licheniformis beta-lactamase, Bacillus stearothermophilus neutral proteases (nprT, nprS, nprM), and Bacillus subtilis prsA. Further signal peptides are known in the art (See e.g., Simonen and Palva, Microbiol. Rev., 1993, 57:109-137). In some embodiments, effective signal peptide coding regions for filamentous fungal host cells include, but are not limited to the signal peptide coding regions obtained from the genes for Aspergillus oryzae TAKA amylase, Aspergillus niger neutral amylase, Aspergillus niger glucoamylase, Rhizomucor miehei aspartic proteinase, Humicola insolens cellulase, and Humicola lanuginosa lipase. Useful signal peptides for yeast host cells include, but are not limited to those from the genes for Saccharomyces cerevisiae alpha-factor and Saccharomyces cerevisiae invertase. Useful signal peptides for mammalian host cells include but are not limited to, those from the genes for immunoglobulin gamma (IgG) and the signal peptide in a human secreted protein, such as human beta-galactosidase polypeptide.

In some embodiments, the control sequence is a regulatory sequence that facilitates the regulation of the expression of the recombinant polynucleotide and/or encoded polypeptide. Examples of regulatory systems are those that cause the expression of the gene to be turned on or off in response to a chemical or physical stimulus, including the presence of a regulatory compound. In prokaryotic host cells, suitable regulatory sequences include, but are not limited to the lac, tac, and trp operator systems. In yeast host cells, suitable regulatory systems include, but are not limited to the ADH2 system or GAL1 system. In filamentous fungi, suitable regulatory sequences include, but are not limited to the TAKA alpha-amylase promoter, Aspergillus niger glucoamylase promoter, and Aspergillus oryzae glucoamylase promoter. Exemplary inducible promoters regulated by exogenous agents include the zinc-inducible sheep metallothionine (MT) promoter, dexamethasone (Dex)-inducible promoter, mouse mammary tumor virus (MMTV) promoter; ecdysone insect promoter, tetracycline-inducible promoter system, RU486-inducible promoter system, and the rapamycin-inducible promoter system.

In another aspect, the present disclosure provides an expression vector comprising a recombinant polynucleotide encoding an RNA ligase polypeptide, where the recombinant polynucleotide is operably or operatively linked to a control sequence, such as a promoter and a terminator, a replication origin, etc., depending on the type of hosts into which they are to be introduced. The recombinant expression vector may be any suitable vector (e.g., a plasmid or virus), that can be conveniently subjected to recombinant DNA procedures and bring about the expression of the RNA ligase polynucleotide sequence. The choice of the vector typically depends on the compatibility of the vector with the host cell into which the vector is to be introduced. The vectors may be linear or closed circular plasmids.

In some embodiments, the expression vector is an autonomously replicating vector (i.e., a vector that exists as an extra-chromosomal entity, the replication of which is independent of chromosomal replication, such as a plasmid, an extra-chromosomal element, a minichromosome, or an artificial chromosome). The vector may contain any means for assuring self-replication. In some alternative embodiments, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used.

In some embodiment, recombinant polynucleotides may be provided on a non-replicating expression vector or plasmid. In some embodiments, the non-replicating expression vector or plasmid can be based on viral vectors defective in replication (see, e.g., Travieso et al., npj Vaccines, 2022, Vol. 7, Article 75).

In some embodiments, the expression vector contains one or more selectable markers, which permit easy selection of transformed cells. A “selectable marker” is a gene, the product of which provides for biocide or viral resistance, resistance to heavy metals, prototrophy to auxotrophs, and the like. Examples of bacterial selectable markers include, but are not limited to the dal genes from Bacillus subtilis or Bacillus licheniformis, or markers, which confer antibiotic resistance such as ampicillin, kanamycin, chloramphenicol or tetracycline resistance. Suitable markers for yeast host cells include, but are not limited to ADE2, HIS3, LEU2, LYS2, MET3, TRP1, and URA3. Selectable markers for use in filamentous fungal host cells include, but are not limited to, amdS (acetamidase; e.g., from A. nidulans or A. orzyae), argB (ornithine carbamoyltransferases), bar (phosphinothricin acetyltransferase; e.g., from S. hygroscopicus), hph (hygromycin phosphotransferase), niaD (nitrate reductase), pyrG (orotidine-5′-phosphate decarboxylase; e.g., from A. nidulans or A. orzyae), sC (sulfate adenyltransferase), and trpC (anthranilate synthase), as well as equivalents thereof.

In another aspect, the present disclosure provides a host cell comprising at least one recombinant polynucleotide encoding an RNA ligase polypeptide of the present disclosure, the recombinant polynucleotide(s) being operatively linked to one or more control sequences for expression of the RNA ligase polypeptide. In some embodiments, the host cells suitable for use in expressing the polypeptides encoded by the expression vectors is a prokaryotic cell or eukaryotic cell known in the art and include but are not limited to, bacterial cells, such as E. coli, Vibrio fluvialis, Streptomyces and Salmonella typhimurium cells; fungal cells, such as yeast cells (e.g., Saccharomyces cerevisiae or Pichia pastoris (ATCC Accession No. 201178)); insect cells such as Drosophila S2 and Spodoptera Sf9 cells; animal (e.g., mammalian) cells such as CHO, COS, BHK, 293, and Bowes melanoma cells; and plant cells. Exemplary host cells also include various Escherichia coli strains (e.g., W3110 (ΔfhuA) and BL21).

In another aspect, the present disclosure provides a method of producing the RNA ligase polypeptides, where the method comprises culturing a host cell comprising an expression vector capable of expressing or producing the RNA ligase polypeptide under suitable culture conditions such that the RNA ligase polypeptide is expressed or produced. In some embodiments, the method comprises isolating the RNA ligase from the culture medium and/or host cell, as described herein. In some further embodiments, the method further comprises purifying the expressed RNA ligase polypeptide.

In some embodiments, the RNA ligase polypeptide expressed in a host cell is recovered from the cells and/or the culture medium using any one or more of the known techniques for protein purification, including, among others, lysozyme or detergent treatment, sonication, filtration, salting-out, ultra-centrifugation, and chromatography, such as described herein.

Chromatographic techniques for isolation/purification of the RNA ligase polypeptides include, among others, reverse phase chromatography, high-performance liquid chromatography, ion-exchange chromatography, hydrophobic-interaction chromatography, size-exclusion chromatography, gel electrophoresis, and affinity chromatography. Conditions for purifying the RNA ligase depends, in part, on factors such as net charge, hydrophobicity, hydrophilicity, molecular weight, molecular shape, etc., and will be apparent to those having skill in the art. In some embodiments, affinity techniques may be used to isolate the RNA ligase. For affinity chromatography purification, an antibody that specifically binds RNA ligase polypeptide may be used. In some embodiments, an affinity tag, e.g., His-tag, can be introduced into the RNA ligase polypeptide for purposes of isolation/purification.

Appropriate culture media and growth conditions for the above-described host cells are well known in the art. Polynucleotides for expression of the RNA ligases may be introduced into cells by various methods known in the art. Techniques include, among others, electroporation, biolistic particle bombardment, liposome mediated transfection, calcium chloride transfection, and protoplast fusion.

In some embodiments, the polynucleotides encoding the RNA ligase polypeptide can be prepared by standard solid-phase methods, according to known synthetic methods. In some embodiments, polynucleotide fragments can be individually synthesized, then joined (e.g., by enzymatic or chemical litigation methods, or polymerase mediated methods) to form any desired continuous sequence (see, e.g., Hughes et al., Cold Spring Harb Perspect Biol. 2017 January; 9 (1): a023812). For example, polynucleotides and oligonucleotides disclosed herein can be prepared by chemical synthesis using the classical phosphoramidite method (See e.g., Beaucage et al., Tetra. Lett., 1981, 22:1859-69; and Matthes et al., EMBO J., 1984, 3:801-05), as it is typically practiced in automated synthetic methods.

In some embodiments, a method for preparing the RNA ligase can comprise: (a) synthesizing a polynucleotide encoding a polypeptide comprising an amino acid sequence selected from the amino acid sequence of an RNA ligase, such as described in the Tables of the Examples, and (b) expressing the engineered RNA ligase encoded by the polynucleotide. In some embodiments of the method, the amino acid sequence encoded by the polynucleotide can optionally have one or several (e.g., up to 3, 4, 5, or up to 10) amino acid residue deletions, insertions and/or substitutions. In some embodiments, the amino acid sequence has optionally 1-2, 1-3, 1-4, 1-5, 1-6, 1-7, 1-8, 1-9, 1-10, 1-15, 1-20, 1-21, 1-22, 1-23, 1-24, 1-25, 1-30, 1-35, 1-40, 1-45, or 1-50 amino acid residue deletions, insertions and/or substitutions. In some embodiments, the amino acid sequence has optionally 1, 2, 3, 4, 5, 6, 7, 8, 9, 10, 11, 12, 13, 14, 15, 16, 17, 18, 19, 20, 21, 22, 23, 24, 25, 30, 30, 35, 40, 45, or 50 amino acid residue deletions, insertions and/or substitutions. In some embodiments, the amino acid sequence has optionally 1, 2, 3, 4, 5, 6, 7, 8, 9, 10, 11, 12, 13, 14, 15, 16, 18, 20, 21, 22, 23, 24, or 25 amino acid residue deletions, insertions and/or substitutions. In some embodiments, the substitutions can be conservative or non-conservative substitutions. The expressed polypeptide can be assessed for the desired property, e.g., RNA ligase activity on one or more oligonucleotide substrates, such as described in the Examples.

### Composition of RNA Ligases

In a further aspect, the present disclosure provides compositions of the engineered RNA ligases disclosed herein. In some embodiments, the composition comprises at least one engineered RNA ligase polypeptide described herein, e.g., an engineered RNA ligase provided in Tables 3, 4, 5, 6, 7.1, 7.2, 8, 9, 10, 11.3, 11.4, and 12, or in the Sequence Listing. In some embodiments, a composition comprises an RNA ligase comprising an amino acid sequence comprising residues 12 to 343 of SEQ ID NO: 2, an amino acid sequence comprising SEQ ID NO: 2, or an amino acid sequence comprising residues 12 to 343 of an even numbered SEQ ID NO. of SEQ ID NOs: 4-220, 224-252, and 270-958, or an amino acid sequence comprising an even numbered SEQ ID NO. of SEQ ID NOs: 4-220, 224-252, and 270-958. In some embodiments, the composition comprises an engineered RNA ligase comprising an amino acid sequence comprising residues 12 to 343 of SEQ ID NO: 4, 106, 218, 286, 396, 436, 520, 552, or 582, or comprising SEQ ID NO: 4, 106, 218, 286, 396, 436, 520, 552, or 582. In some embodiments, the engineered RNA ligase polypeptide in the compositions is isolated or purified. In some embodiments, the RNA ligase is combined with other components and compounds to provide compositions and formulations comprising the engineered RNA ligase polypeptide as appropriate for different applications and uses.

In some embodiments, the composition further comprises one or more of a buffer, a nucleotide substrate (e.g., ATP or dATP), and/or at least one or more polynucleotide ligase substrates. In some embodiments, the polynucleotides substrates are single stranded or double stranded, or comprises single stranded and double stranded polynucleotide substrates. In some embodiments, the polynucleotide substrate comprises at least two oligonucleotide fragments, where an oligonucleotide fragment comprises double stranded oligonucleotide fragment and the at least two double stranded oligonucleotide fragments can base pair to serve as substrates for the RNA ligase. In some embodiments, the composition further comprises at least 2, 3, 4, 5, 6 or more double stranded oligonucleotide fragments, each of which can serve as substrates for the RNA ligase. In some embodiments, the polynucleotide substrate is a modified polynucleotide. In some embodiments, the modified polynucleotide comprises a modification on the sugar residue, e.g., 2′-position, a modified phosphate group, e.g., phosphorothioate; or modified nucleobase, or any combination of modifications, as further described herein.

In some embodiments, the RNA ligase substrate concentration in the composition is at about 0.01-1 mM, 0.05-0.9 mM, 0.1-0.8 mM, 0.2-0.7 mM, or 0.3 mM-0.6 mM. In some embodiments, the RNA ligase substrate is at about 0.01 mM, 0.05 mM, 0.1 mM, 0.2 mM, 0.3 mM, 0.4 mM, 0.5 mM, 0.6 mM, 0.7 mM, 0.8 mM, 0.9 mM or 1 mM. In some embodiments, the RNA ligase substrate concentration is at about 1-10 mM, 2-8 mM, or 4-6 mM. In some embodiments, the engineered RNA ligase exhibits increased product yield at about 1 mM, 2 mM, 3 mM, 4 mM, 5 mM, 6 mM, 7 mM, 8 mM, 9 mM, or 10 mM.

In some embodiments, the composition further comprises a nucleotide cofactor or substrate used by the RNA ligase to catalyze the joining reaction. In some embodiments, the nucleotide cofactor or substrate is ATP and/or dATP. Preferably, the nucleotide cofactor or substrate is ATP. In some embodiments, the nucleotide cofactor or substrate (e.g., ATP) is present at a concentration of about 0.05-25 mM, 1-20 mM, 2-18 mM, or 5-15 mM. In some embodiments, the nucleotide cofactor or substrate is present at a concentration of about 0.5 mM, 1 mM, 2 mM, 3 mM 4 mM, 5 mM, 6 mM, 7 mM, 8 mM, 9 mM, 10 mM, 12 mM, 15 mM, 20 mM, or 25 mM.

In some embodiments, the composition further comprises a buffer, In some embodiments, the buffer comprises, among others, borate, phosphate, 2-(N-morpholino) ethanesulfonic acid (MES), 3-(N-morpholino) propanesulfonic acid (MOPS), acetate, triethanolamine (TEoA), and 2-amino-2-hydroxymethyl-propane-1,3-diol (Tris), and the like. In some embodiments, the buffer concentration is from 1 to 200 mM, 5 to 200 mM, 1 to 150 mM, 5 to 150 mM, 1 to 100 mM, 5 to 100 mM, 1 to 50 mM, 5 to 50 mM, 1 to 20 mM, 5 to 20 mM, 1 to 10 mM, or 5 to 10 mM.

In some embodiments, the composition further comprises an additive or ligation enhancing agent, including, among others, one or more of DMSO, betaine, polyethylene glycol (e.g., PEG 6000, PEG 8000, etc.), bovine serum albumin, Ficoll, and dextran (e.g., Dextran 6000). In some embodiments, the composition comprises 1% to 40% v/v of DMSO. In some embodiments, the composition comprises 0.1 M to 3 M betaine. In some embodiments, the composition comprises 0.5% to 20% w/v of PEG (e.g., PEG6000 or PEG8000).

In some embodiments, the composition further comprises an RNase inhibitor, including, among others, porcine RNase inhibitor, human placental RNase inhibitor, human liver RNase inhibitor, mouse RNase inhibitor, rat RNase inhibitor, and recombinantly expressed RNase inhibitors thereof.

In some embodiments, an engineered RNA ligase described herein is provided in solution or is immobilized on a substrate. In some embodiments, the substrate is a solid substrate or a membrane or particles. The enzyme can be entrapped in matrixes or membranes. In some embodiments, matrices include polymeric materials such as calcium-alginate, agar, k-carrageenin, polyacrylamide, and collagen, or solid matrices, such as activated carbon, porous ceramic, and diatomaceous earth. In some embodiments, the matrix is a particle, a membrane, or a fiber. Types of membranes include, among others, nylon, cellulose, polysulfone, or polyacrylate.

In some embodiments, the enzyme is immobilized on the surface of a support material. In some embodiments, the enzyme is adsorbed on the support material. In some embodiments, the enzyme is immobilized on the support material by covalent attachment. Support materials include, among others, inorganic materials, such as alumina, silica, porous glass, ceramics, diatomaceous earth, clay, and bentonite, or organic materials, such as cellulose (CMC, DEAE-cellulose), starch, activated carbon, polyacrylamide, polystyrene, and ion-exchange resins, such as Amberlite, Sephadex, and Dowex.

### Uses and Methods

In another aspect, the present disclosure provides uses of the engineered RNA ligases for polynucleotide synthesis, RNA repair, or other molecular biological uses.

In some embodiments, the engineered RNA ligase is used for ligating polynucleotides and oligonucleotides. In some embodiments, the engineered RNA ligase is used for synthesizing polynucleotides from shorter oligonucleotides. In some embodiments, a method of ligating at least a first polynucleotide strand and a second polynucleotide strand comprises contacting a first polynucleotide strand and a second polynucleotide strand with an engineered RNA ligase described herein in presence of a nucleotide substrate under conditions suitable for ligation of the first polynucleotide strand to the second polynucleotide strand, wherein the first polynucleotide strand comprises a ligatable 5′-end and the second polynucleotide strand comprises a 3′-end ligatable to the 5′-end of the first polynucleotide strand.

In some embodiments, the second polynucleotide strand comprising a ligatable 3′-end has at least 2, 3, 4, 5 or 6 ribonucleotides at the 3′ terminal end. In some embodiments of the method, the 3′-end of the second polynucleotide strand is a 3′-OH. In some embodiments of the method, the 5′-end of the first polynucleotide strand is a 5′-phosphate. In some embodiments of the method, the internucleoside linkage contained in the first polynucleotide strand and/or second polynucleotide strand comprises a phosphate linkage.

In some embodiments, the first polynucleotide strand has at least a 5′-terminal nucleotide with a 2′-modified sugar moiety. In some embodiments, at least 2, 3, 4, or more of nucleotides at the 5′-terminal region of the first polynucleotide strand has 2′-modified sugar moiety. In some embodiments, the first polynucleotide strand has at least a 5′-terminal nucleotide with a non-natural internucleoside linkage to the adjacent 3′-nucleotide. In some embodiments, at least 2, 3, 4 or more nucleotides at the 5′-terminal region of the first polynucleotide strand has a non-natural internucleoside linkage. In some embodiments, the non-natural linkage is a phosphorothioate internucleoside linkage.

In some embodiments, the second polynucleotide strand has at least the 3′-terminal nucleotide with a 2′-modified sugar moiety. In some embodiments, at least 2, 3, 4, or more of nucleotides at the 3′-terminal region of the second polynucleotide strand have a 2′-modified sugar moiety. In some embodiments, the second polynucleotide strand has at least a 3′-terminal nucleotide with a non-natural internucleoside linkage to the adjacent 5′-nucleotide. In some embodiments, at least 2, 3, 4 or more nucleotides at the 3′-terminal region of the second polynucleotide strand has a non-natural internucleoside linkage. In some embodiments, the non-natural linkage is a phosphorothioate internucleoside linkage.

In some embodiments, the first polynucleotide strand has at least a 5′-terminal nucleotide with a 2′-modified sugar moiety, and the second polynucleotide strand has at least a 3′-terminal nucleotide with a 2′-modified sugar moiety. In some embodiments, at least 2, 3, 4, or more of nucleotides at the 5′-terminal region of the first polynucleotide strand have 2′-modified sugar moieties, and at least 2, 3, 4, or more of nucleotides at the 3′-terminal region of the second polynucleotide strand have 2′-modified sugar moieties. As further discussed below, in some embodiments, the 2′-modified sugar moiety is a 2′-O-methyl, 2′-O-ethyl, or 2′-fluoro. In some embodiments, the first polynucleotide strand has at least a 5′-terminal nucleotide with a non-natural internucleoside linkage to the adjacent 3′-nucleotide, and the second polynucleotide strand has at least a 3′-terminal nucleotide with a non-natural internucleoside linkage to the adjacent 5′-nucleotide. In some embodiments, at least 2, 3, 4 or more nucleotides at the 5′-terminal region of the first polynucleotide strand and at least 2, 3, 4 or more nucleotides at the 3′-terminal region of the second polynucleotide strand have a non-natural internucleoside linkage. In some embodiments, the non-natural internucleoside linkage is a phosphorothioate internucleoside linkage.

In some embodiments, the method further comprising a third polynucleotide strand, wherein the first polynucleotide strand and second polynucleotide strand hybridize adjacent to one another on the third polynucleotide strand to position the 5′-end of the first polynucleotide strand adjacent to the 3′-end of the second polynucleotide strand. In some embodiments of the method, the third polynucleotide strand is continuous with the first polynucleotide strand or second polynucleotide strand. In some embodiments of the method, the third polynucleotide strand is continuous with the first polynucleotide strand and second polynucleotide strand to form a single continuous polynucleotide ligase substrate. In some embodiments, the third polynucleotide strand is RNA, DNA, or a mixture of RNA and DNA.

In some embodiments of the method, the third polynucleotide comprises a splint or bridging polynucleotide, wherein the 5′-terminal sequence of the first polynucleotide strand and the 3′-terminal sequence of the second polynucleotide strand hybridize adjacent to one another on the splint or bridging polynucleotide to position the 5′-end of the first polynucleotide strand adjacent to the 3′-end of the second polynucleotide strand. In some embodiments, the splint or bridging polynucleotide is RNA, DNA, or a mixture of RNA and DNA.

In some embodiments, the second polynucleotide strand comprising a ligatable 3′-end has at least 4, 5 or 6 ribonucleotides at the 3′ terminal end. In some embodiments of the method, the 3′-end of the second polynucleotide strand is a 3′-OH. In some embodiments of the method, the 5′-end of the first polynucleotide strand is a 5′-phosphate. In some embodiments of the method, the internucleoside linkage contained in the first polynucleotide strand and/or second polynucleotide strand comprises a phosphate linkage.

In some embodiments of the method, the third polynucleotide strand comprises a splint or bridging polynucleotide, wherein the 5′-terminal sequence of the first polynucleotide strand and the 3′-terminal sequence of the second polynucleotide strand hybridize adjacent to one another on the splint or bridging polynucleotide to position the 5′-end of the first polynucleotide strand adjacent to the 3′-end of the second polynucleotide strand.

In some embodiments, one or more additional polynucleotide strands can be hybridized to the third polynucleotide strand, where an additional polynucleotide strand hybridizes adjacent to the 3′-end of the first polynucleotide strand, or adjacent to the 5′-end of the second polynucleotide strand, or two additional polynucleotide strands hybridize adjacent to the 3′-end of the first polynucleotide strand and adjacent to the 5′-end of the second polynucleotide strand to form nick(s) that can be ligated with an engineered RNA ligase. Additional polynucleotide strands can be hybridized in like manner and ligated to prepare longer polynucleotide strands.

In some embodiments, the first polynucleotide strand and/or second polynucleotide strand can have a single strand region (e.g., overhang or unpaired regions) when hybridized to the third polynucleotide strand, and a fourth polynucleotide strand added that hybridizes to the single stranded region of the first polynucleotide strand or second polynucleotide stranded, where the fourth polynucleotide strand hybridizes adjacent to the 5′-end or 3′-end of the third polynucleotide strand to form a nick, which can be then ligated with an engineered RNA ligase. In some embodiments, the fourth polynucleotide strand can have a single stranded region (e.g., overhang or unpaired regions) when hybridized to the first polynucleotide strand or second polynucleotide strand on the third polynucleotide strand, and a fifth polynucleotide strand added that hybridizes to the single stranded region of the fourth polynucleotide strand, where the fifth polynucleotide strand hybridizes adjacent to the 3′-end of the first polynucleotide strand or the 5′-end of the second polynucleotide strand to form a nick, which can be ligated with an engineered RNA ligase. Such successive additions of polynucleotide strands can be used to generate longer polynucleotide products (see, e.g., Paul et al., ACS Chem. Biol. 2023, 18, 2183-2187, incorporated by reference herein).

In some embodiments, the first polynucleotide strand is hybridized to the third polynucleotide strand to form a first double stranded fragment, and the second polynucleotide strand is hybridized to a fourth polynucleotide strand to form a second double stranded fragment, where the first and second double stranded fragments can base pair to form a substrate for the engineered RNA ligase. In some embodiments, the first double stranded fragment and the second double stranded fragment have complementary overhangs or complementary single stranded ends, also referred to as cohesive or sticky ends, that can base pair and form double stranded nick(s) between the first and second double stranded fragments that serve as a substrate for the engineered RNA ligase.

In some embodiments, the complementary ends (sticky or cohesive ends) are of sufficient length for the double stranded fragments to base pair and form suitable substrates for the engineered RNA ligase. In some embodiments, the complementary single stranded end comprises at least 1-50, 2-40, 3-35, 4-30, 5-25, 6-20, or 8-15 or more nucleotides in length. In some embodiments, the complementary single stranded end is from 1-10, 2-8, or 4-6 nucleotides in length. In some embodiments, the complementary single stranded end is 1, 2, 3, 4, 5, 6, 8, 10, 12, 14, 16, 18, 20, or more nucleotides in length.

In some embodiments, the double stranded region or complementary portion of each of the double stranded fragment is from 4-50, 6-45, 8-40, 10-35, 12-30, or 14-25 nucleotides in length. In some embodiments, the double stranded region is at least 4, 5, 6, 7, 8, 9, 10, 12, 14, 16, 18, 20, 22, 24, 26, 28, 30, 35, 40, 45, or 50 nucleotides in length.

In some embodiments of the method, the ligase substrates comprise at least 2, 3, 4, 5, or 6 or more double stranded fragments, where each double stranded fragment has a complementary end that can base pair with at least one other double stranded fragment with a complementary end to form substrates for the RNA ligase. In some embodiments, the double stranded fragments have complementary or cohesive ends, e.g., at the 5′ and 3′ ends, such that the double stranded fragments can be ligated to form concatemers. In some embodiments, where the number of double stranded fragments is 3, at least one of the double stranded fragments has complementary ends at the 5′ and 3′ ends of the fragment, where double stranded fragment base pairs with two other different double stranded fragments to form a substrate that can be ligated to a product containing the three double stranded fragments. It is to be understood that where 3 or more double stranded fragments are ligated, the complementary ends of the double stranded fragments can be designed for ligation of all of the different double stranded fragments.

In some embodiments, the polynucleotide substrate comprises one or more modified nucleotides. In some embodiments, the modification comprises a modified sugar residue, a modified nucleobase, and/or modified phosphate group. In some embodiments, the modified sugar residue is modified at the 2-position of the sugar moiety. In some embodiments, the modified 2-position is a 2′-halo or 2′-O-alkyl, preferably a lower C₁-C₄alkyl, e.g., methyl or ethyl. In some embodiments, the modified nucleotide is a modified phosphate group, for example a phosphorothioate group. In some embodiments, the modified phosphate group is at the 5′-end of a polynucleotide substrate. In some embodiments, the modified phosphate group is at an internucleoside linkage of the polynucleotide substrate. In some embodiments, the modified nucleotide has a modified nucleobase. In some embodiments, the polynucleotide substrate includes an inverted nucleotide or an abasic nucleotide (e.g., 5′-5′ linkage or a 3′-3′ linkage), including 3′-3′ inverted dA, dT, dG, dC, dU, or abasic deoxyribonucleotide; and/or 5′-5′ inverted dA, dT, dG, dC, dU, or abasic deoxyribonucleotide.

In some embodiments of the method, the first polynucleotide strand and/or second polynucleotide strand comprise one or more modified nucleotides or nucleotide analogs, wherein the modified nucleotide or nucleotide analog comprises a nucleobase analog, modified nucleoside sugar residue, modified internucleoside linkage, modified 5′-end phosphate group, and/or modified 3′-end hydroxyl group.

In some embodiments, the polynucleotide ligase substrate comprises a modified 5′-end phosphate group, wherein the modified phosphate group is a phosphate analog. In some embodiments of the method, the first polynucleotide comprises a 5′-phosphate analog. In some embodiments, the 5′-phosphate analog is a phosphorothioate, phosphoramidate, monomethylphosphate, methylphosphonate, vinylphosphonate, or phosphonocarboxylate.

In some embodiments of the method, the polynucleotide ligase substrate comprises one or more modified nucleoside sugar residues. In some embodiments of the method, the first polynucleotide and/or second polynucleotide comprises one or more modified nucleoside sugar residues. In some embodiments, the modified nucleoside sugar residue is a 2′-O-alkyl, a 2′-halo, a β-D-ribo LNA, or a α-L-ribo-LNA (locked nucleic acids). In some embodiments, the modified nucleoside sugar residue is, among others, a 2′-O-methyl, 2′-O-ethyl, or 2′-O-propyl group. In some embodiments, the modified nucleoside sugar residue is 2′-fluoro, 2′-bromo, or 2′-chloro, preferably 2′-fluoro. In some embodiments, the sugar residue is modified with a ligand, such as a cell targeting ligand, or a cell penetrating moiety, for example, GalNAc and lipid moieties. In some embodiments, the sugar residue is modified with a linker moiety.

In some embodiments of the method, the polynucleotide ligase substrate comprises one or more modified nucleotide residues having a modified nucleobase or nucleobase analog. In some embodiments of the method, the first polynucleotide and/or second polynucleotide comprises one or more modified nucleotide residues having a modified nucleobase or nucleobase analog. In some embodiments, the nucleobase analog is, among others, xanthine, hypoxanthine, inosine, 6-methyladenine, 7-methylguanine, 2,6-diaminopurine, 5-methylcytosine, 5-hydroxycytosine, 5-bromocytosine, 5-iodocytosine, 2-thiothymine, 5-fluorouracil, 5-bromouracil, 8-bromoguanine, 8-aminoguanine, or 8-aza-7-deazaguanine. In some embodiments, the nucleobase is modified with a ligand, such as a chemical label, a cell targeting ligand, or a cell penetrating moiety. In some embodiments, the nucleobase is modified with linker moiety, for example for conjugating a ligand.

In some embodiments of the method, the polynucleotide ligase substrate comprises one or more modified or non-standard internucleoside linkages, i.e., internucleoside linkages other than phosphate linkages. In some embodiments, the first polynucleotide and/or second polynucleotide comprises one or more modified or non-standard internucleoside linkages. In some embodiments, the internucleoside linkage is a phosphorothioate, phosphoacetate, phosphoramidate, methylphosphonate, or phosphonocarboxylate. In some embodiments, at least 1%, 2%, 5%, 10%, 20%, 30%, 40%, 50%, 60%, 70%, 80%, 90%, or more of the internucleoside linkages are non-standard internucleoside linkages. In some embodiments, the non-standard internucleoside linkages are present 1, 2, 3, 4 or 5 nucleotides of the 5′ and/or 3′-terminus of the polynucleotide substrates. In some embodiments, the non-standard internucleoside linkage, such as phosphorothioate, is diastereomeric, e.g., S or P configuration.

In some embodiments of the method, the polynucleotide ligase substrate comprises modified 3′-hydroxyl group. In some embodiments, 3′-end of a polynucleotide ligase substrate is attached to a matrix or surface, such as a solid matrix. In such instances, the polynucleotide ligase substrate attached to a matrix has a ligatable 5′-end. In some embodiments, the 3′-end of a ligase substrate is modified with an amine, halo, phosphate, phosphate analog, —O-alkyl, lipid moiety, or a detectable label. In some embodiments, the 5′-end of the polynucleotide substrate is modified with a cell-targeting, cell penetrating, or linker moiety. In some embodiments, the cell targeting moiety is a GalNAc moiety.

In some embodiments, the polynucleotide ligase substrate comprises C-4′ modifications, including among others, 4′-thio-C2′ modifications, 4′/5′ aminoalkyl/C2′ modifications, C4′-Guanidino-C2′-modifications, and C4′-O-Me/C2′-modifications (see, e.g., Gangopadhyay et al., RNA Biology, 2022, 19:1, 452-467).

In some embodiments, the engineered RNA ligase is used in a method to synthesize RNA or DNA/RNA polynucleotides by ligation of shorter RNA or DNA/RNA oligonucleotides. In some embodiments, the engineered RNA ligase is used to ligate the 3′ OH of RNA to the 5′ phosphate of DNA or RNA. In some embodiments, the engineered RNA ligase is used to ligate the 3′ OH of RNA to the 5′ phosphate of DNA in a double-stranded-format NGS RNA library construction. In some embodiments, the engineered RNA ligase is used in a method of preparing RNA rings. In some embodiments, the engineered RNA ligase is used to repair nicks in dsRNA or dsRNA/DNA. In some embodiments, the engineered RNA ligase is used to synthesize modified oligonucleotides, such as shRNA and antisense oligonucleotides.

In some embodiments, the amount of engineered RNA ligase used is sufficient to ligate the oligonucleotide substrates or ligate a nick formed by the oligonucleotide substrates to the desired level. The amount of enzyme used can also be adjusted based the activity of the ligase, and type of substrate being ligated. In some embodiments, the amount of enzyme used is about 0.1 mg/mL to about 5 mg/mL. about 0.5 mg/mL to about 4 mg/mL, 1 mg/mL to about 3 mg/mL, or about 1.5 to about 2.5 mg/mL. In some embodiments, the engineered RNA ligase is provided at concentrations from about 0.01 g/L to about 50 g/L; about 0.01 to about 0.1 g/L; about 0.05 g/L to about 50 g/L; about 0.1 g/L to about 40 g/L; about 1 g/L to about 40 g/L; about 2 g/L to about 40 g/L; about 5 g/L to about 40 g/L; about 5 g/L to about 30 g/L; about 0.1 g/L to about 10 g/L; about 0.5 g/L to about 10 g/L; about 1 g/L to about 10 g/L; about 0.1 g/L to about 5 g/L; about 0.5 g/L to about 5 g/L; or about 0.1 g/L to about 2 g/L.

In some embodiments, the method includes a nucleotide substrate used by the RNA ligase to catalyze the joining reaction. In some embodiments, the nucleotide substrate is ATP and/or dATP. Preferably, the nucleotide substrate is ATP. In some embodiments, the reaction conditions for the ligation include additional components, such as Mg⁺², buffer and/or salts.

In some embodiments, the nucleotide cofactor or substrate (e.g., ATP) is present at a concentration of about 0.05-25 mM, 1-20 mM, 2-18 mM, or 5-15 mM. In some embodiments, the nucleotide cofactor or substrate is present at a concentration of about 0.5 mM, 1 mM, 2 mM, 3 mM 4 mM, 5 mM, 6 mM, 7 mM, 8 mM, 9 mM, 10 mM, 12 mM, 15 mM, 20 mM, or 25 mM.

In some embodiments, the buffer comprises, among others, borate, phosphate, 2-(N-morpholino) ethanesulfonic acid (MES), 3-(N-morpholino) propanesulfonic acid (MOPS), acetate, triethanolamine (TEoA), and 2-amino-2-hydroxymethyl-propane-1,3-diol (Tris), and the like. In some embodiments, the buffer concentration is from 1 to 200 mM, 5 to 200 mM, 1 to 150 mM, 5 to 150 mM, 1 to 100 mM, 5 to 100 mM, 1 to 50 mM, 5 to 50 mM, 1 to 20 mM, 5 to 20 mM, 1 to 10 mM, or 5 to 10 mM.

In some embodiments, the reaction conditions also include a ligation enhancing reagent, including, among others, polyethylene glycol (e.g., PEG 6000 and PEG 8000) or other molecular crowding reagents, for example bovine serum albumin (BSA), dextran, and Ficoll.

In some embodiments, ligation reaction is carried out at a suitable temperature and reaction time period. In some embodiments, the ligation reaction temperature is from about 2° C. to about 60° C. In some embodiments, the ligation reaction temperature is from 4° C. to 55° C., 4° C. to 50° C., 4° C. to 45° C., or 10° C. to 40° C. In some embodiments, the ligation reaction temperature is 2° C., 5° C., 10° C., 15° C., 20° C., 25° C., 30° C., 37° C., 40° C., 45° C., 50° C., 55° C., or 60° C. In some embodiments, the ligation reaction temperature can use different temperatures, for example a temperature at which stable hybrids are formed between polynucleotide substrate(s) followed by a higher temperature to promote completion of ligation reaction.

In some embodiments, the ligation reaction time can be a sufficient time for ligation of polynucleotide substrate(s). In some embodiments, the ligation reaction time is from 0.5-72 hr or longer. In some embodiments, the ligation reaction time is 1-72 hr, 2-48 hr, or 2-24 hr. In some embodiments, the ligation reaction time is 0.5, 1, 2, 4, 5, 12, 24, 48, or 72 hr or longer.

In some embodiments, the ligase reaction is carried out at a suitable pH. In some embodiments, the pH of the ligase reaction is about pH 5-9, about pH 5.5-8.5, about pH 6-8, or about pH 6.5-7.5. In some embodiments, the pH of the ligase reaction is about pH 5, about pH 5.5, about pH 6, about pH 6.5, about pH 7, about pH 7.5, about pH 8, about pH 8.5 or about pH 9.

In some embodiments, the RNA ligase substrate concentration is provided at a concentration of about 0.05 to about 25 mM, 0.1-20 mM, 0.1-15 mM, 1-10 mM, 2-8 mM, or 4-6 mM. In some embodiments, substrate concentration is at about 0.01-1 mM, 0.05-0.9 mM, 0.1-0.8 mM, 0.2-0.7 mM, or 0.3 mM-0.6 mM. In some embodiments, the substrate concentration is at about 0.01 mM, 0.05 mM, 0.1 mM, 0.2 mM, 0.3 mM, 0.4 mM, 0.5 mM, 0.6 mM, 0.7 mM, 0.8 mM, 0.9 mM or 1 mM, 1.5 mM, 2 mM, 2.5 mM, 3 mM, 3.5 mM, 4 mM, 4.5 mM, or 5 mM, or any appropriate concentration for efficient ligation of the substrate.

In some embodiments, the polynucleotide substrate is bound to a support, and the ligation reaction carried out with the engineered RNA ligase to ligate a polynucleotide substrate to the support bound polynucleotide, or ligate one or more polynucleotide substrates hybridized to the support-bound polynucleotide substrate.

In some embodiments, the ligase reaction further includes a nucleotide substrate regeneration system to regenerate co-factor substrate ATP (or dATP). In some embodiments, the ATP recycling or regeneration system includes a nucleoside monophosphate kinase for converting AMP to ADP in presence of a phosphate donor. Various nucleoside monophosphate kinase can be used for the conversion of AMP to ADP, including homologs of nucleoside monophosphate kinases. In some embodiments, more than one nucleoside monophosphate kinase can be used in the regeneration system. In some embodiments, the nucleoside monophosphate kinase is an adenosine monophosphate kinase (e.g., adenylate kinase), cytidine monophosphate (CMP) kinase, uridine monophosphate (UMP) kinase, or guanylate-monophosphate (GMP) kinase.

In some embodiments, a nucleoside monophosphate kinase useful in the enzymatic reactions is a cytidine monophosphate kinases. Various suitable cytidine monophosphate kinases are known in the art. These include homologs of cytidine monophosphate kinases. In some embodiments, a cytidine monophosphate kinase useful in the enzymatic reactions includes, among others, the cytidine monophosphate kinase of Thermus thermophilus (Q5SL35), Pyrococcus furiosus (Q8U2L4), Pseudomonas putida (AFO48857.1), Escherichia coli K-12 MG1655 (POA610), Clostridium acetobutylicum (Q97108), Halobacterium salinarum (Q9HPA5) Bacillus acidicola (WP_066270173), Acetobacter aceti (WP_010667744), Acidithiobacillus thiooxidans (WP_024892761.1), Acidithiobacillus ferrooxidans (WP_064220349.1), Metallosphaera sedula (WP_011921264.1), Amphibacillus xylanus (WP_015009966.1) Thioalkalivibrio denitrificans (WP_077278466.1), Vibrio psychroerythus (Q482G4), Pseudoalteromonas haloplanktis (Q3ILA1), Psychrobacter arcticus (Q4FRL5), Psychromonas ingrahamii (A1SZ01), Pseudomonas syringae (xQ4ZQ97) and Halobacterium salinarum (Q9HPA5).

In some embodiments, a nucleoside monophosphate kinase useful in the enzymatic reactions is a uridine monophosphate kinase. Various suitable uridine monophosphate kinases are known in the art. These include homologs of uridine monophosphate kinases. In some embodiments, a uridine monophosphate kinase useful in the enzymatic reactions includes, among others, the uridine monophosphate kinase of Pyrococcus furiosus (Q8U122), Thermus thermophilus (P43891), Pseudomonas putida (I7BW46), Escherichia coli K-12 MG1655 (P0ATE9), Aspergillus niger (A2R195), Saccharomyces cerevisiae (P15700), Clostridium acetobutylicum (Q97164) ATCC 824 PyrH Halobacterium salinarum (Q9HNN8), Picrophilus torridus (WP_048059653), Metallosphaera sedula (WP_012021705), Thermoplasma acidophilum (WP_010900913), Sulfolobus solfataricus (WP_009992427), Acetobacter aceti (WP_042788648), Thioalkalivibrio sp. HK1 (WP_081759172.1), Amphibacillus xylanus (WP_015010200.1), Vibrio psychroerythus (Q485G8), Pseudoalteromonas haloplanktis (Q3IIX6), Psychrobacter arcticus (Q4FRH5), Psychromonas ingrahamii (ABM04676.1), Pseudomonas syringae (Q4ZWS6), and Halobacterium salinarum (Q9HNN8).

In some embodiments, the nucleoside monophosphate kinase useful in the enzymatic reactions is a guanosine monophosphate kinase (guanylate kinase). Various suitable guanylate kinases are known in the art. These include homologs of guanylate kinases. In some embodiments, a guanylate kinase useful in the enzymatic reactions includes, among others, the guanylate kinase of Thermotoga maritima (Q9X215), Thermus thermophilus (Q5SI18), Pseudomonas putida (17C087), Escherichia coli K-12 (P60546), Aspergillus niger (A2QPV2), Saccharomyces cerevisiae (P15454), Clostridium acetobutylicum (Q97IDO), Acidithiobacillus ferrooxidans (WP_064219869.1), Acidithiobacillus thiooxidans (WP_010637919.1), Bacillus acidicola (WP_066264774.1), Acetobacter aceti (WP_018308252.1), Amphibacillus xylanus (WP_015010280.1), Thioalkalivibrio sulfidiphilus (WP_018953989.1), Vibrio psychroerythus (Q47UB3), Pseudoalteromonas haloplanktis (Q3IJH8), Psychrobacter arcticus (Q4FQY7), Psychromonas ingrahamii (A1T0P1), and Pseudomonas syringae (Q4ZZY8).

In some embodiments, the nucleoside monophosphate kinase useful in the enzymatic reactions is an adenosine monophosphate kinase (adenylate kinase). Various suitable adenylate kinases are known in the art. These include homologs of adenylate kinases. In some embodiments, the adenylate kinase is a bacterial, fungal, plant, or animal adenylate kinase. In some embodiments, an adenylate kinase useful in the enzymatic reactions includes, among others, adenylate kinases of Thermus thermophilus (Q72125), Pyrococcus furiosus (Q8U207), Pseudomonas putida (17CAA9), Escherichia coli K-12 W3110 (P69441), Aspergillus niger CBS 513.88 (A2QPN9), Saccharomyces cerevisiae (P07170), Clostridium acetobutylicum (Q97E39), Halobacterium salinarum (Q9HPAT), Acidithiobacillus thiooxidans (WP_024894015.1), Acidithiobacillus ferrooxidans (WP_064218420.1), Bacillus acidicola (WP_066267988.1), Sulfolobus solfataricus (WP_009991241.1), Saccharomyces cerevisiae (P07170), Thermotoga neapolitana (Q8GGL2), Escherichia coli (P69441) and Geobacillus stearothermophilus (WP_049624206.1). In some embodiments, the adenylate kinase is an engineered adenylate kinase described in International patent application No. PCT/US2024/051084, filed Oct. 11, 2024, incorporated herein by reference.

In some embodiments, the ATP regeneration system includes at least an enzyme for the conversion of ADP to ATP in presence of a phosphate donor. In some embodiments, the ATP regeneration system includes, among others, an acetate kinase, pyruvate kinase, creatine kinase, or polyphosphate kinase (see, e.g., Endo et al., Adv. Synth. Catal., 2002, 343:521-526; Andexer et al., Chem Bio Chem., 2015, 16:380-386). In the ATP regeneration system, the phosphate donor for the conversion of ADP to ATP is selected based on the ATP regenerating enzyme employed. By way of example and not limitation, if acetate kinase enzyme is used for conversion of ADP to ATP, the phosphate donor is acetyl-phosphate. If pyruvate kinase is used for the conversion of ADP to ATP, the phosphate donor is phosphoenolpyruvate. If creatine kinase is used for the conversion of ADP to ATP, the phosphate donor is creatine phosphate. If polyphosphate kinase is used for the conversion of ADP to ATP, the phosphate donor is inorganic polyphosphate.

Accordingly, in some embodiments, the ATP regenerating system includes pyruvate kinase and phosphoenolpyruvate. In some embodiments, the ATP regenerating system includes creatine kinase and creatine phosphate. In some embodiments, the ATP regenerating system includes polyphosphate kinase and inorganic polyphosphate. In some embodiments, the ATP regenerating system includes acetate kinase and acetyl phosphate. In some embodiments, the acetate kinase is an acetate kinase of Escherichia coli str. K-12 substr. MG1655 (NP_416799.1), Corynebacterium jeikeium K411 (WP_011272972.1), Lactococcus cremoris subsp. cremoris KW2 (WP_011835968.1), Lactococcus lactis (WP_004254593.1), Marinitoga sp. 38H-ov (WP_165147355.1), Thermotoga sp. KOL6 (WP_101510533.1), Thermosipho melaniensis (WP_012057479.1), Thermotoga sp. RQ7 (WP_041844042.1), and Thermosipho africanus (WP_004102380.1). In some embodiments, the acetate kinase is an engineered acetate kinase described in International patent application No. PCT/US2024/051118, filed Oct. 11, 2024, incorporated by reference herein.

In some embodiments, the engineered RNA ligase is used to ligate nicks or related nick structures (see, e.g., Cheng et al., Royal Soc Chem Adv., 2019, 9:8620-8627). In some embodiments, the engineered RNA ligase is used to synthesize RNAs from shorter oligonucleotides, for example by use of splint nucleic acids (see, e.g., Stark et al., RNA, 2006, 12:2014-2019). In some embodiments, the engineered RNA ligase is used to ligate modified oligonucleotides, such as provided in the Examples. Other examples of modified oligonucleotide products that can be synthesized using short oligonucleotide substrates, include, among others, shRNAs or siRNAs described in patent publications WO22104366, WO22029209, WO22031847, WO20226960, US2022072024, US2021238606, U.S. Pat. No. 11,286,488, US2017305956, WO22212153, WO22192519, WO22125490, WO22072447, WO21257568, WO21102373, WO21072395, WO21022108, US2022079971, U.S. Pat. No. 11,034,957, US2021332365, U.S. Pat. Nos. 11,015,201, 10,995,336, 11,091,759, 10,889,813, 10,130,651, 10,513,703, WO19232255, WO21108640, WO22147304, and WO21138537.

In a further aspect, the present disclosure provides a kit comprising an RNA ligase described herein. In some embodiments, the kit further comprises a buffer, a nucleotide substrate (e.g., ATP or dATP), and/or one or more polynucleotide ligase substrates. In some embodiments, the kit further comprises an additive or ligation enhancing agent, including one or more of polyethylene glycol (e.g., PEG 6000, PEG 8000, etc.) or other molecular crowding reagents, bovine serum albumin, Ficoll, dextran (e.g., Dextran 6000).

The following Examples, including experiments and results achieved, are provided for illustrative purposes only and are not to be construed as limiting the present invention.

### EXAMPLES

In the experimental disclosure below, the following abbreviations where relevant apply: ppm (parts per million); M (molar); mM (millimolar), uM and μM (micromolar); nM (nanomolar); mol (moles); gm and g (gram); mg (milligrams); ug and μg (micrograms); L and 1 (liter); ml and mL (milliliter); ul, μl, uL, μL (microliter); cm (centimeters); mm (millimeters); um and μm (micrometers); sec. (seconds); min(s) (minute(s)); h(s) and hr(s) (hour(s)); U (units); OD (optical density); MW (molecular weight); rpm (rotations per minute); rcf (relative centrifugal force); psi and PSI (pounds per square inch); ° C. (degrees Celsius); RT and rt (room temperature); NGS (next-generation sequencing); ds (double stranded); ss (single stranded); CDS (coding sequence); DNA (deoxyribonucleic acid); RNA (ribonucleic acid); E. coli W3110 (commonly used laboratory E. coli strain, available from the Coli Genetic Stock Center [CGSC], New Haven, CT); HTP (high throughput); HPLC (high pressure liquid chromatography); FPLC (fast protein liquid chromatography); PBS (phosphate buffered saline); BSA (bovine serum albumin); DTT (dithiothreitol); CAM (chloramphenicol); CAT (chloramphenicol acetyltransferase); IPTG (isopropyl β-D-1-thiogalactopyranoside); FIOPC (fold improvements over positive control); FIOP (fold improvements over parent); LB (Luria-Bertani); TB (Terrific-Broth).

### Example 1

E. coli Expression Hosts Containing Recombinant RNA Ligase 2 Genes

To produce enzyme, RNA ligase 2 genes were cloned in the pJV110900 vector system (see U.S. Pat. No. 10,184,117B2) operatively linked to the lac promoter under control of the lacI repressor. The expression vector also contains the P15a origin of replication and the chloramphenicol (CAM) resistance gene. The resulting plasmid was transformed into E. coli W3110, using standard methods known in the art. The transformants were isolated by subjecting the cells to CAM selection, as known in the art (See e.g., U.S. Pat. No. 8,383,346 and WO2010/144103).

### Example 2

### Preparation of HTP RNA Ligase 2 Containing Wet Cell Pellets

E. coli cells containing recombinant RNA ligase 2-encoding genes from monoclonal colonies were inoculated into 180 μL LB growth media containing 1% glucose and 30 μg/mL CAM in the wells of 96-well, shallow-well microtiter plates. The plates were sealed with air permeable seals, and cultures were grown overnight at 30° C., 200 rpm, and 85% humidity. Then, 10 μL of each of the cell cultures were transferred into the wells of 96-well, deep-well plates containing 390 mL TB growth media and 30 μg/mL CAM. The deep-well plates were sealed with air-permeable seals and incubated at 30° C., 250 rpm, and 85% humidity until an OD₆₀₀ of 0.6-0.8 was reached. The cell cultures were then induced by IPTG at a final concentration of 1 mM and incubated overnight under the same conditions prior to induction. The cells were then pelleted using centrifugation at 4,000 rpm for 10 min. The supernatants were discarded, and the pellets were frozen at −80° C. prior to lysis.

### Example 3

E. coli Shake Flake Expression and Purification of Recombinant RNA Ligase 2

To characterize improved enzyme variants after each round of evolution, shake flask production and purification was performed. HTP cultures grown, as described above, were thawed and 30 μL from each HTP culture was used to inoculate 1 L shake flasks containing: 160 mL Terrific Broth (TB) media (Teknova), 30 μg/mL chloramphenicol, 0.03% (v/v) lactose and 0.075% (v/v) glucose. The shake flasks were grown at 32° C. for 18 hours at 250 rpm. Following this incubation period, the cultures were centrifuged at 4,000 rpm for 10 min. The culture supernatant was discarded, and the pellets were resuspended in 30 mL of 50 mM Tris-HCl, pH 7.5. The cell suspension was chilled in an ice bath and lysed using a Microfluidizer cell disruptor (Microfluidics M-110L). The crude lysate was pelleted by centrifugation (10,000 rpm for 90 min at 4° C.), and the supernatant was then filtered through a 0.2 μm PES membrane to further clarify the lysate. The clarified lysates were then supplemented with 20 mM imidazole and 500 mM NaCl. The lysates were then purified using an AKTA Pure purification system and a 5 mL HisTrap FF column (Cytiva) using the run parameters provided below (Table 1). The wash buffer comprised of 50 mM Tris-HCl pH 8.0, 500 mM NaCl, 20 mM imidazole, 0.02% Triton X-100, and the elution buffer comprised of 50 mM Tris-HCl pH 8.0, 500 mM NaCl, 250 mM imidazole and 0.02% Triton X-100 in the following:

TABLE 1
FPLC run parameters

| Parameter | Volume | Units |
|---|---|---|
| Column volume | 5 | mL |
| Flow rate | 12 | mL/min |
| Pressure limit pre-column | 0.5 | MPa |
| Pressure limit delta-column | 0.3 | MPa |
| Sample volume | 50 | mL |
| Equilibration volume | 5 column volumes | |
| | (CV) = 25 mL | |
| Wash Unbound volume | 20 CV = 100 mL | |
| Elution | Isocratic (step) | |
| Elution volume | 5 CV = 25 mL | |
| Fraction volume | 1.5 | mL |
| RE-equilibration volume | 5 CV = 25 mL | |

The most concentrated fractions were identified by UV absorption (280 nm) and pooled; 3 ml of eluate was dialyzed overnight in 1× ligase storage buffer (40 mM Tris-HCl pH 7.5, 100 mM KCl, 0.1 mM EDTA, and 50% glycerol) in a 3K Slide-A-Lyzer™ dialysis cassette (Thermo Fisher) for buffer exchange. RNA ligase 2 concentrations from the preparations were measured by reading the absorption at 280 nm.

The purified RNA ligase 2 proteins were then screened by observing antisense strand (AS) conversion, sense strand (SS) conversion, and total conversion using the analytical method described in Example 15 and the results are shown in Tables 2.2 to 2.3.

### Example 4

### Measurement of Activity of Wild Type RNA Ligase 2 Samples

To determine the round one backbone, a set of nine enzymes were selected based on their homology to SEQ ID NO: 254. To test their activity, 0.27 g/L, 0.13 g/L, 0.07 g/L, 0.03 g/L, and 0.0033 g/L of each tested protein was assayed in reactions containing 20 g/L of the shortmer mix A, where each oligonucleotide is at 1.2 mM (Table 2.1) and 26.6% (v/v) reaction buffer. The reactions were incubated for 20 hours at 33° C. in a Multitron incubator with shaking at 700 rpm. Afterwards, EDTA was added to each reaction to a final concentration of 10 mM and the samples analyzed via HPLC to quantify the remaining fragment concentrations and product yields.

TABLE 2.1
Shortmer mix A components
Shortmer description

| | |
|---|---|
| 1 | U*-Af*-C*-Uf-G-Af-U |
| 2 | 5′-p-Cf-A-Af-A-Uf-A-Uf-G-Uf-U-Gf-A-Gf*-C |
| 3 | Tri-NAG*-invAb*-G-C-U-C-A-A-C-A |
| 4 | 5′-p-Uf-Af-Uf-U-U-G-A-U-C-A-G-U-A*-invAb |

In the above description of oligonucleotides, “-” represents a phosphate linkage; “*-” represents a phosphorothioate linkage; nucleotide N (e.g., A, G, U, C) represents a 2′-methoxy (2′-O-methyl) modified nucleotide; Nf represents a 2′-fluoro modified nucleotide; Tri-NAG represents a tri-antennary N-Acetyl galactosamine (GalNAc) ligand; invAb represents an inverted abasic residue (inverted abasic deoxyribonucleotide); and 5′-p represents 5′-phosphate. Further information regarding the full oligonucleotide construct can be found, for example, in U.S. Pat. No. 10,995,335 to Arrowhead Pharmaceuticals, Inc.

Oligonucleotide 1 base pairs with oligonucleotide 4, and oligonucleotide 2 base pairs with oligonucleotides 3 and 4. Oligonucleotides 1-4 when hybridized form a nick between the 3′-end of oligonucleotide 1 and 5-end of oligonucleotide 2, and a nick between the 3′-end of oligonucleotide 3 and the 5′-end of oligonucleotide 4. Ligation of oligonucleotides 1 and 2 results in formation of the anti-sense strand, and ligation of oligonucleotides 3 and 4 results in formation of the sense strand.

Percent conversions were calculated as in the following using analytical method in Example 13 and the results are shown in Tables 2.2 to 2.4:

$$Antisense A strand conversion : \% conversion = \frac{Area under the curve for anti sense strand product SEQ I D 557}{Total area under the curve for all products and substrates} * 100 \%$$

$$Sense strand A conversion : \% conversion = \frac{Area under the curve for sense strand product SEQ I D 556}{Total area under the curve for all products and substrates} * 100 \%$$

$$Total A conversion : Sum of antisense A strand conversion and sense A strand conversion .$$

TABLE 2.2
RNA Ligase sense A strand conversion relative to SEQ ID NO: 254

| | | Pos Control FIOP Relative to SEQ ID NO: 254 | | | | |
|---|---|---|---|---|---|---|
| | | (Sense Strand Conversion) | | | | |
| SEQ ID NO: | | 0.27 | 0.13 | 0.03 | 0.07 | 0.003 |
| (nt/aa) | Organism | g/L | g/L | g/L | g/L | g/L |
| 1/2 | Escherichia phage RB69 | ++ | ++ | ++ | ++ | ++++ |
| 255/256 | Escherichia phage vB_EcoM JS09 | + | + | | | |
| 257/258 | Escherichia phage Bp7 | ++ | ++ | ++ | ++ | |
| 259/260 | Escherichia phage vB_EcoM VR7 | ++ | ++ | ++ | ++ | + |
| 261/262 | Enterobacter phage PG7 | ++ | ++ | ++ | ++ | + |
| 263/264 | Salmonella phage S16 (Salmonella phage | ++ | ++ | ++ | ++ | |
| | vB_SenM-S16) | | | | | |
| 265/266 | Acinetobacter phage ZZ1 | ++ | ++ | ++ | ++ | |
| 267/268 | Klebsiella phage KP27 | ++ | + | + | + | |
| 221/222 | Aeromonas phage CC2 | ++ | ++ | ++ | ++ | |

Levels of increased activity were determined relative to the reference polypeptide of SEQ ID NO: 254 and defined as follows: “+” >0.15 “++” >0.85, “+++” >1.2, “++++” >2.

TABLE 2.3
RNA Ligase antisense A strand conversion relative to SEQ ID NO: 254

| | | Pos Control FIOP Relative to SEQ ID NO: 254 | | | | |
|---|---|---|---|---|---|---|
| | | (Antisense A Strand Conversion) | | | | |
| SEQ ID NO: | | 0.27 | 0.13 | 0.03 | 0.07 | 0.003 |
| (nt/aa) | Organism | g/L | g/L | g/L | g/L | g/L |
| 1/2 | Escherichia phage RB69 | +++ | +++ | +++ | +++ | |
| 255/256 | Escherichia phage vB_EcoM JS09 | + | | | | |
| 257/258 | Escherichia phage Bp7 | +++ | ++ | ++ | ++ | |
| 259/260 | Escherichia phage vB_EcoM_VR7 | +++ | +++ | +++ | +++ | |
| 261/262 | Enterobacter phage PG7 | +++ | +++ | +++ | +++ | |
| 263/264 | Salmonella phage S16 (Salmonella | +++ | +++ | +++ | +++ | |
| | phage vB_SenM-S16) | | | | | |
| 265/266 | Acinetobacter phage ZZ1 | ++ | ++ | + | + | |
| 267/268 | Klebsiella phage KP27 | ++++ | ++++ | ++ + | ++++ | |
| 221/222 | Aeromonas phage CC2 | ++++ | ++++ | ++++ | ++++ | |

Levels of increased activity were determined relative to the reference polypeptide of SEQ ID NO: 254 and defined as follows: “+” >0.15 “++” >0.85, “+++” >1.2, “++++” >2.

TABLE 2.4
RNA Ligase A total conversion relative to SEQ ID NO: 254

| | | Pos Control FIOP Relative to SEQ ID NO: 254 | | | | |
|---|---|---|---|---|---|---|
| | | (Total A Conversion) | | | | |
| SEQ ID NO: | | 0.27 | 0.13 | 0.03 | 0.07 | 0.003 |
| (nt/aa) | Organism | g/L | g/L | g/L | g/L | g/L |
| 1/2 | Escherichia phage RB69 | ++ | ++ | ++ | ++ | ++++ |
| 255/256 | Escherichia phage | + | | | | |
| | vB_EcoM_JS09 | | | | | |
| 257/258 | Escherichia phage Bp7 | ++ | ++ | ++ | ++ | |
| 259/260 | Escherichia phage | ++ | ++ | ++ | ++ | + |
| | vB_EcoM_VR7 | | | | | |
| 261/262 | Enterobacter phage PG7 | ++ | ++ | ++ | ++ | + |
| 263/264 | Salmonella phage S16 | ++ | ++ | ++ | ++ | |
| | (Salmonella phage vB_SenM- | | | | | |
| | S16) | | | | | |
| 265/266 | Acinetobacter phage ZZ1 | ++ | ++ | ++ | ++ | |
| 267/268 | Klebsiella phage KP27 | ++ | ++ | ++ | ++ | |
| 221/222 | Aeromonas phage CC2 | ++ | ++ | ++ | ++ | |

Levels of increased activity were determined relative to the reference polypeptide of SEQ ID NO: 254 and defined as follows: “+” >0.15 “++” >0.85, “+++” >1.2, “++++” >2.

The RNA ligase of SEQ ID NO: 2 was selected as the round one backbone due to it displaying the highest activity at low enzyme load (e.g. 0.0033 g/L).

### Example 5

### Improvements Over SEQ ID NO: 2 in RNA Ligase 2 Activity

The RNA ligase of SEQ ID NO: 2 was selected as the parent enzyme after screening for activity as described in Example 4. Libraries of engineered genes were produced using known techniques (e.g., saturation mutagenesis, recombination of previously identified beneficial mutations, etc.). The polypeptides encoded by each gene were produced in HTP as described in Example 1, and the cell paste was generated as described in Example 2.

To prepare cells for lysis, 300 μL of 0.1 mg/mL lysozyme in 50 mM TRIS-HCl buffer at pH 7.5 was added to the cell paste of each sample. After thorough resuspension, the cells were incubated at 41° C. for 1 hour in a PCR thermocycler. The samples were then centrifuged for 10 minutes at 4,000 rpm and 4° C. and the clarified supernatants were used in subsequent biocatalytic reactions.

To screen the enzyme variants, lysate, 10% (v/v) final concentration, was transferred into 96-well shallow well plates containing 20 g/L of the shortmer mix (Table 2.1) and 26.6% (v/v) reaction buffer. The reactions were incubated for 20 hours at 33° C. in a Multitron (Infors) with shaking at 700 rpm. Afterwards, EDTA was added to each reaction to a final concentration of 10 mM and the samples analyzed via HPLC to quantify the remaining fragment concentrations and product yields.

Using peak areas determined using the analytical method described in Example 13, the percent conversion for each sample was calculated as described in Example 4. The conversion results are shown in Table 3. Activity relative to SEQ ID NO: 2 (FIOP) was calculated as the percent conversion formed by the variant over percent conversion of SEQ ID NO: 2 and shown in Table 3.

TABLE 3
RNA ligase activity for total A conversion,
antisense A strand conversion, and sense A
strand conversion relative to SEQ ID NO: 2

| | | | Pos Control | |
|---|---|---|---|---|
| | Amino Acid | | FIOP Anti | Pos Control |
| SEQ | Differences | Pos Control | Sense | FIOP Sense |
| ID NO: | (Relative to | FIOP Total | A Strand | A Strand |
| (nt/aa) | SEQ ID NO: 2) | AConversion | Conversion | Conversion |
| 3/4 | L179F | ++ | +++ | ++ |
| 5/6 | A184P | ++ | +++ | ++ |
| 7/8 | E196K | ++ | +++ | ++ |
| 9/10 | T92A | ++ | +++ | ++ |
| 11/12 | V40E | ++ | +++ | ++ |
| 13/14 | V82I | ++ | +++ | ++ |
| 15/16 | Y32R | ++ | +++ | ++ |
| 17/18 | I207T | ++ | +++ | ++ |
| 19/20 | T33K | ++ | +++ | ++ |
| 21/22 | G114K | ++ | +++ | ++ |
| 23/24 | S171E | ++ | +++ | ++ |
| 25/26 | T33A | ++ | +++ | ++ |
| 27/28 | F221E | ++ | +++ | ++ |
| 29/30 | I207S | ++ | +++ | ++ |
| 31/32 | F202W | ++ | ++ | ++ |
| 33/34 | R101G | ++ | ++ | ++ |
| 35/36 | A184R | ++ | ++ | ++ |
| 37/38 | N141C | ++ | ++ | ++ |
| 39/40 | N60R | ++ | ++ | ++ |
| 41/42 | A184L | ++ | ++ | ++ |
| 43/44 | Y98R | ++ | ++ | + |
| 45/46 | F96S | + | ++ | + |
| 47/48 | N205V | + | ++ | + |
| 49/50 | S191K | + | ++ | + |
| 51/52 | D193N | + | ++ | + |
| 53/54 | N141A | + | ++ | + |
| 55/56 | N320K | + | ++ | + |
| 57/58 | M174Q | + | ++ | + |
| 59/60 | Y98D | + | ++ | + |
| 61/62 | L71K | + | ++ | + |
| 63/64 | T142R | + | ++ | + |
| 65/66 | E57T | + | ++ | + |
| 67/68 | N320D | + | + | + |
| 69/70 | N205R | + | ++ | + |
| 71/72 | D209L | + | ++ | + |
| 73/74 | T145D | + | ++ | + |
| 75/76 | K14R | + | ++ | + |
| 77/78 | D209E | + | ++ | + |
| 79/80 | V342I | + | ++ | + |
| 81/82 | P255E | + | + | + |
| 83/84 | V327Q | + | ++ | + |
| 85/86 | D170E | + | ++ | + |
| 87/88 | N141E | + | + | + |
| 89/90 | Y98P | + | ++ | + |
| 91/92 | I339K | + | ++ | + |
| 93/94 | T38D | + | + | + |
| 95/96 | T142H | + | + | + |
| 97/98 | D75Q | + | + | + |
| 99/100 | A184M | + | + | + |
| 101/102 | V342T | + | + | |
| 103/104 | I339R | | ++ | |

Levels of increased activity were determined relative to the reference polypeptide of SEQ ID NO: 2 and defined as follows: “+” >1, “++” >1.2, “+++” >2.

### Example 6

### Improvements Over SEQ ID NO: 4 in RNA Ligase 2 Activity

The engineered RNA ligase of SEQ ID NO: 4 was selected as the parent enzyme after screening variants as described in Example 5 above. Libraries of engineered genes were produced using known techniques (e.g., saturation mutagenesis, recombination of previously identified beneficial mutations, etc.). The polypeptides encoded by each gene were produced in HTP as described in Example 1 and the cell paste was generated as described in Example 2.

The variants were prepared and screened as described in Example 5, except 600 μL was used as the lysis volume. The conversion results are shown in Table 4. Activity relative to SEQ ID NO: 4 (FIOP) was calculated as the percent conversion formed by the variant over the percent conversion of SEQ ID NO:

TABLE 4
RNA ligase activity for total A conversion, antisense A strand conversion,
and sense A strand conversion relative to SEQ ID NO: 4

| | | | Pos Control | Pos Control |
|---|---|---|---|---|
| SEQ ID | Amino Acid | Pos Control | FIOP Anti | FIOP Sense |
| NO: | Differences | FIOP Total A | Sense A Strand | A Strand |
| (nt/aa) | (Relative to SEQ ID NO: 4) | Conversion | Conversion | Conversion |
| 105/106 | T33A/R101G/F221E/N320D/R335E/P336S/ | + | +++ | + |
| | 1339R | | | |
| 107/108 | L71K/T92A/S171E/A184P/F202W | + | +++ | + |
| 109/110 | V40E/T92A/S171E/F202W/V327Q | + | ++ | + |
| 111/112 | E196K/Q300K/R335E/I339R | + | ++ | + |
| 113/114 | T33A/A184R/F221E/Q300K/N320D | + | ++ | + |
| 115/116 | R335E/P336S/I339R | + | ++ | + |
| 117/118 | A184P/E196K/F221E/P336S | + | +++ | + |
| 119/120 | T33K/A184R/N320D/R335E/P336S | + | ++ | + |
| 121/122 | I207T/D209L/F221E/Q300K/N320D/P336S/ | + | +++ | + |
| | I339R | | | |
| 123/124 | R101G/E196K/F221E/Q300K/R335E | + | ++ | + |
| 125/126 | I207T/D209L/Q300K/N320D | + | ++ | + |
| 127/128 | V40E/D170E/S171E/F202W/V327Q | + | +++ | + |
| 129/130 | T33K/R101G/A184R/E196K/D209L/P336S/ | + | ++ | + |
| | I339R | | | |
| 131/132 | V40E/L71K/T92A/S171E/A184P/V327Q | + | ++ | + |
| 133/134 | I207T/R335E/P336S/I339R | + | ++ | + |
| 135/136 | T33K/R101G/E196K/Q300K/N320D/R335E/ | + | ++ | + |
| | P336S/I339R | | | |
| 137/138 | E57T/S171E/F179L/A184P/F202W/V327Q | + | ++ | + |
| 139/140 | E57T/T92A/F202W/V327Q | + | ++ | + |
| 141/142 | T33K/R101G/I207T/F221E/Q300K/R335E/ | + | ++ | + |
| | P336S/I339R | | | |
| 143/144 | T33K/F221E/P336S | + | ++ | + |
| 145/146 | E196K/F221E/Q300K/R335E/P336S/I339R | + | ++ | + |
| 147/148 | T33K/A184P/F221E/P336S/I339R | + | +++ | + |
| 149/150 | T33A/A184P/E196K/F221E/Q300K | + | ++ | + |
| 151/152 | R101G/E196K/I207T/D209L/F221E/Q300K/ | + | +++ | + |
| | P336S/I339R | | | |
| 153/154 | R101G/A184R/R335E/P336S/I339R | + | ++ | + |
| 155/156 | T33A/A184L/E196K/N320D/P336S/I339R | + | ++ | + |
| 157/158 | T33A/A184P/E196K/Q300K/N320D/R335H/ | + | +++ | + |
| | P336S/I339R | | | |
| 159/160 | A184L/N320D/R335E/P336S/I339R | + | ++ | + |
| 161/162 | A184L/E196K/I207T/D209L/Q300K/R335E/ | + | ++ | + |
| | P336S/I339R | | | |
| 163/164 | A184R/E196K/N320D/R335E/P336S/I339R | + | ++ | + |
| 165/166 | T33K/E196K/D209L/F221E/I339R | + | ++ | + |
| 167/168 | A184R/N320D/R335E/P336S/I339R | + | ++ | + |
| 169/170 | R101G/D209L/R335E/P336S/I339R | + | ++ | + |
| 171/172 | R101G/E196K/D209L/I339R | + | ++ | + |
| 173/174 | V40E/L71K/V82I/D170E/S171E/F179L/ | + | ++ | + |
| | F202W/V327Q | | | |
| 175/176 | N320D/R335E/P336S/I339R | + | ++ | + |
| 177/178 | R101G/A184L/Q300K/R335E/P336S/I339R | + | ++ | + |
| 179/180 | T33K/R101G/E196K/D209L/R335E/P336S/ | + | ++ | + |
| | I339R | | | |
| 181/182 | T33K/E196K/Q300K/N320D | + | ++ | + |
| 183/184 | F221E/R335E | + | ++ | + |
| 185/186 | T33K/A184R/E196K/I207S/F221E | + | ++ | + |
| 187/188 | F221E/R335E/P336S/I339R | + | ++ | + |
| 189/190 | E196K/D209L/Q300K/R335E/P336S/I339R | + | ++ | + |
| 191/192 | A184P/E196K/Q300K/N320D | + | ++ | + |
| 193/194 | E57T/S171E/F202W/V327Q | + | ++ | + |
| 195/196 | V40E/L71K/S171E/A184P | + | ++ | + |
| 197/198 | A184L/E196K/F221E | + | ++ | + |
| 199/200 | T33A/E196K/I207S/R335E/P336S | + | ++ | + |
| 201/202 | Y32R/E57T/L71K/F202W/V327Q/V342I | + | ++ | + |
| 203/204 | T33A/E196K/F221E/P336S | + | ++ | + |
| 205/206 | R101G/F221E/Q300K/N320D/R335E/ | + | ++ | + |
| | P336S/I339R | | | |
| 207/208 | T33K/A184P/N320D/R335H/P336S | + | ++ | + |
| 209/210 | T33K/R101G/I207S/F221E | + | ++ | + |
| 211/212 | R101G/A184R/E196K/D209L/F221E | + | ++ | + |
| 213/214 | T33A/I207S/D209L/Q300K/R335E/P336S/ | + | ++ | + |
| | I339R | | | |
| 215/216 | T33K/L83P/A184P/E196K/Q300K/P336S | + | ++ | + |

Levels of increased activity were determined relative to the reference polypeptide of SEQ ID NO: 4 and defined as follows: “+” >1, “++” >1.2, “+++” >2.

### Example 7

### Improvements Over SEQ ID NO: 106 in RNA Ligase 2 Activity

The engineered RNA ligase of SEQ ID NO: 106 was selected as the parent enzyme after screening variants as described in Example 6 above. Libraries of engineered genes were produced using known techniques (e.g., saturation mutagenesis, recombination of previously identified beneficial mutations, etc.). The polypeptides encoded by each gene were produced in HTP as described in Example 1 and the cell paste was generated as described in Example 2.

The variants were prepared and screened as described in Example 5, except 1200 μL was used as the lysis volume. The conversion results are shown in Table 5. Activity relative to SEQ ID NO: 106 (FIOP) was calculated as the percent conversion formed by the variant over percent conversion of SEQ ID NO: 106.

TABLE 5
RNA ligase activity for total A conversion, antisense A strand conversion,
and sense A strand conversion relative to SEQ ID NO: 106

| | | | Pos Control | Pos Control |
|---|---|---|---|---|
| | Amino Acid | Pos Control | FIOP Anti Sense | FIOP Sense |
| SEQ ID NO: | Differences | FIOP Total A | A Strand | A Strand |
| (nt/aa) | (Relative to SEQ ID NO: 106) | Conversion | Conversion | Conversion |
| 217/218 | T38D/L71K/G114N/A184M/S191K/ | + | ++ | + |
| | E196K | | | |
| 219/220 | N141A/D170E/S171E/M174Q | + | ++ | + |
| 223/224 | G114R | + | ++ | + |
| 225/226 | E57T/N60R/D170E/S171E/M174Q/ | + | ++ | + |
| | E192V/E196K/D320K | | | |
| 227/228 | N60R/T92A/D170E/S171E | + | ++ | + |
| 229/230 | N141A/M174Q/E196K | + | ++ | + |
| 231/232 | E57T/D170E/S171E/M174Q | + | ++ | + |
| 233/234 | N141T/A184P | + | ++ | + |
| 235/236 | E57T/N60R/N141A/D170E/S171E/ | + | ++ | + |
| | M174Q/E192V | | | |
| 237/238 | N60R/D170E/M174Q | + | ++ | + |
| 239/240 | N141C/D170E/S171E | + | ++ | + |
| 241/242 | P69L/L71K/G114N/A184M | + | ++ | |
| 243/244 | T38D/V40E | + | ++ | + |
| 245/246 | V40E/L71K/Y98R/A184M/I259V | + | ++ | + |
| 247/248 | Y32R/N60R/N141A/D170E/S171E/ | + | ++ | + |
| | M174Q/E196K/D320K | | | |
| 249/250 | Y32R/D170E/I207T/D320K | + | + | + |
| 251/252 | E196K | + | + | + |

Levels of increased activity were determined relative to the reference polypeptide of SEQ ID NO: 106 and defined as follows: “+” >1, “++” >1.2.

### Example 8

### Improvements Over SEQ ID NO: 218 in RNA Ligase 2 Activity

The engineered RNA ligase of SEQ ID NO: 218 was selected as the parent enzyme after screening for activity as described in Example 7. Libraries of engineered genes were produced using known techniques (e.g., saturation mutagenesis, recombination of previously identified beneficial mutations, etc.). The polypeptides encoded by each gene were produced in HTP as described in Example 1, and the cell paste was generated as described in Example 2.

To prepare cells for lysis, 600 μL of 0.1 mg/mL lysozyme in 50 mM TRIS-HCl buffer at pH 7.5 was added to the cell paste of each sample. After thorough resuspension, the cells were incubated at 43° C. for 1 hour in a PCR thermocycler. The samples were then centrifuged for 10 minutes at 4,000 rpm and 4° C. and the clarified supernatants were used in subsequent biocatalytic reactions.

To screen the enzyme variants, lysate, 1.25 or 2.5% (v/v) final concentration, was transferred into 96-well shallow well plates containing 20 g/L of the shortmer mix A (Table 2.1) and 26.6% (v/v) reaction buffer. The reactions were incubated for 4 hours at 33° C. in a Multitron (Infors) with shaking at 700 rpm. Afterwards, EDTA was added to each reaction to a final concentration of 10 mM and the samples analyzed via HPLC to quantify the remaining fragment concentrations and product yields.

Using peak areas determined using the analytical method described in Example 15, the percent conversion for each sample was calculated as described in Example 4. The conversion results are shown in Table 6. Activity relative to SEQ ID NO: 218 (FIOP) was calculated as the percent conversion formed by the variant over percent conversion of SEQ ID NO: 218 and shown in Table 6.

TABLE 6
RNA ligase activity for total A conversion,
antisense A strand conversion, and sense A
strand conversion relative to SEQ ID NO: 218

| | | | Pos Control | |
|---|---|---|---|---|
| | Amino Acid | | FIOP Anti | Pos Control |
| SEQ | Differences | Pos Control | Sense | FIOP Sense |
| ID NO: | (Relative to | FIOP Total A | A Strand | A Strand |
| (nt/aa) | SEQ ID NO: 218) | Conversion | Conversion | Conversion |
| 269/270 | A102T | ++ | ++ | ++ |
| 271/272 | S17P | ++ | +++ | ++ |
| 273/274 | A190F | ++ | ++ | ++ |
| 275/276 | Y16R | ++ | ++ | ++ |
| 277/278 | G68W | | ++ | |
| 279/280 | Y23M | | + | |
| 281/282 | Y270A | | +++ | |
| 283/284 | Q117K/T229A | | +++ | |
| 285/286 | Q117W | | +++ | |
| 287/288 | Q117T | ++ | ++ | ++ |
| 289/290 | Q117H | + | ++ | + |
| 291/292 | Q117A | ++ | ++ | ++ |
| 293/294 | Q117M | | +++ | |
| 295/296 | Q117G | +++ | +++ | +++ |
| 297/298 | Q117I | ++ | + | ++ |
| 299/300 | Q117K | + | +++ | |
| 301/302 | Q117R | | +++ | |
| 303/304 | Q117S | ++ | ++ | ++ |
| 305/306 | Q117V | +++ | | +++ |
| 307/308 | Q117F | | +++ | |
| 309/310 | Q117C | +++ | ++ | +++ |
| 311/312 | Q117L | ++ | ++ | ++ |

Levels of increased activity were determined relative to the reference polypeptide of SEQ ID NO: 218 and defined as follows: “+” >1, “++” >1.2, “+++” >2

### Example 9

### Improvements Over SEQ ID NO: 286 in RNA Ligase 2 Activity

The engineered RNA ligase of SEQ ID NO: 286 was selected as the parent enzyme after screening for activity as described in Example 8. Libraries of engineered genes were produced using known techniques (e.g., saturation mutagenesis, recombination of previously identified beneficial mutations, etc.). The polypeptides encoded by each gene were produced in HTP as described in Example 1, and the cell paste was generated as described in Example 2.

To prepare cells for lysis, 600 μL of 0.1 mg/mL lysozyme in 50 mM TRIS-HCl buffer at pH 7.5 was added to the cell paste of each sample. After thorough resuspension, the cells were incubated at 43° C. for 1 hour in a PCR thermocycler. The samples were then centrifuged for 10 minutes at 4,000 rpm and 4° C. and the clarified supernatants were used in subsequent biocatalytic reactions.

To screen the enzyme variants, lysate, 2.5% (v/v) final concentration, was transferred into 96-well shallow well plates containing 20 g/L of the shortmer mix A (Table 2.1) and 26.6% (v/v) reaction buffer. The reactions were incubated for 4 hours at 33° C. in a Multitron (Infors) with shaking at 700 rpm. Afterwards, EDTA was added to each reaction to a final concentration of 10 mM and the samples analyzed via HPLC to quantify the remaining fragment concentrations and product yields.

Using peak areas determined using the analytical method described in Example 15, the percent conversion for each sample was calculated as described in Example 4. The conversion results are shown in Table 7.1 and 7.2. Activity relative to SEQ ID NO: 286 (FIOP) was calculated as the percent conversion formed by the variant over percent conversion of SEQ ID NO: 286 and shown in Table 7.1 and 7.2.

TABLE 7.1
RNA ligase activity for total A conversion,
antisense A strand conversion, and sense A
strand conversion relative to SEQ ID NO: 286

| | | | Pos Control | |
|---|---|---|---|---|
| | Amino Acid | | FIOP Anti | Pos Control |
| SEQ | Differences | Pos Control | Sense | FIOP Sense |
| ID NO: | (Relative to | FIOP Total A | A Strand | A Strand |
| (nt/aa) | SEQ ID NO: 286) | Conversion | Conversion | Conversion |
| 313/314 | G296E | +++ | +++ | ++ |
| 315/316 | N114V/W117K | +++ | +++ | ++ |
| 317/318 | K26S | +++ | +++ | ++ |
| 319/320 | K30C | +++ | +++ | ++ |
| 321/322 | W117K/K118G | ++ | +++ | ++ |
| 323/324 | N114P/W117K | ++ | +++ | ++ |
| 325/326 | N114S/W117K | ++ | +++ | ++ |
| 327/328 | G296L | ++ | +++ | ++ |
| 329/330 | W117K/G119S | ++ | ++ | +++ |
| 331/332 | K26P | ++ | ++ | ++ |
| 333/334 | K14F | ++ | ++ | ++ |
| 335/336 | N114G/W117K | ++ | +++ | + |
| 337/338 | N114F/W117K | ++ | ++ | ++ |
| 339/340 | W117K/G119A | ++ | ++ | ++ |
| 341/342 | L183V | ++ | ++ | ++ |
| 343/344 | K26R | ++ | ++ | ++ |
| 345/346 | W117K | ++ | ++ | + |
| 347/348 | G296R | ++ | ++ | ++ |
| 349/350 | A185S | ++ | ++ | ++ |
| 351/352 | K26F | ++ | ++ | ++ |
| 353/354 | W117K/K118L | ++ | ++ | ++ |
| 355/356 | S307A | ++ | ++ | ++ |
| 357/358 | K30R | ++ | ++ | ++ |
| 359/360 | M12S | ++ | ++ | ++ |
| 361/362 | V93A/N135T | ++ | ++ | ++ |
| 363/364 | W117K/K118Q | ++ | ++ | + |
| 365/366 | A185H | ++ | ++ | ++ |
| 367/368 | N135Q | ++ | ++ | + |
| 369/370 | T92M | + | ++ | + |
| 371/372 | P288A | + | ++ | + |
| 373/374 | T92N | + | + | + |
| 375/376 | K14W | + | ++ | + |
| 377/378 | W117K/G119T | + | | ++ |
| 379/380 | T92S | + | + | + |
| 381/382 | T92G | + | + | + |
| 383/384 | W117K/V120P | + | | ++ |
| 385/386 | Y79W | + | ++ | + |
| 387/388 | A100R | + | + | + |
| 389/390 | T287A | + | + | + |

Levels of increased activity were determined relative to the reference polypeptide of SEQ ID NO: 286 and defined as follows: “+” >1, “++” >1.2, “+++” >2

TABLE 7.2
RNA ligase activity for total A conversion,
antisense A strand conversion, and sense A
strand conversion relative to SEQ ID NO: 286

| | | | Pos Control | |
|---|---|---|---|---|
| | Amino Acid | | FIOP Anti | Pos Control |
| SEQ | Differences | Pos Control | Sense A | FIOP Sense |
| ID NO: | (Relative to | FIOP Total A | Strand | A Strand |
| (nt/aa) | SEQ ID NO: 286) | Conversion | Conversion | Conversion |
| 391/392 | W117K/G119E | ++ | ++ | ++ |
| 393/394 | G296V | ++ | ++ | + |
| 395/396 | G292E | +++ | +++ | ++ |
| 397/398 | K26H | ++ | ++ | ++ |
| 399/400 | G296W | ++ | ++ | ++ |
| 401/402 | K30H | +++ | +++ | +++ |
| 403/404 | K26G | +++ | +++ | +++ |
| 405/406 | K30A | +++ | +++ | +++ |
| 407/408 | S25W | +++ | +++ | +++ |
| 409/410 | S25L | +++ | +++ | +++ |
| 411/412 | K26V | +++ | +++ | +++ |
| 413/414 | K26L | +++ | +++ | +++ |
| 415/416 | T92E | ++ | ++ | ++ |
| 417/418 | K14T | +++ | +++ | +++ |
| 419/420 | D75T | ++ | ++ | ++ |
| 421/422 | S25G | ++ | ++ | ++ |
| 423/424 | V327W | ++ | ++ | ++ |
| 425/426 | F111L | | | ++ |

Levels of increased activity were determined relative to the reference polypeptide of SEQ ID NO: 286 and defined as follows: “+” >1, “++” >1.2, “+++” >2

### Example 10

### Improvements Over SEQ ID NO: 396 in RNA Ligase 2 Activity

The engineered RNA ligase of SEQ ID NO: 396 was selected as the parent enzyme after screening variants as described in Example 9 above. Libraries of engineered genes were produced using known techniques (e.g., saturation mutagenesis, recombination of previously identified beneficial mutations, etc.). The polypeptides encoded by each gene were produced in HTP as described in Example 1 and the cell paste was generated as described in Example 2.

To prepare cells for lysis, 600 μL of 0.1 mg/mL lysozyme in 50 mM TRIS-HCl buffer at pH 7.5 was added to the cell paste of each sample. After thorough resuspension, the cells were incubated at 43° C. for 1 hour in a PCR thermocycler. The samples were then centrifuged for 10 minutes at 4,000 rpm and 4° C. and the clarified supernatants were used in subsequent biocatalytic reactions.

To screen the enzyme variants, 2.5% (v/v) final concentration was transferred into 96-well shallow well plates containing 20 g/L of the shortmer mix A (Table 2.1) and 26.6% (v/v) reaction buffer. Lysate was transferred into 96-well shallow well plates containing 20 g/L of the shortmer mix (Table 2.1) and 26.6% (v/v) reaction buffer. The reactions were incubated for 4 hours at 33° C. in a Multitron (Infors) with shaking at 700 rpm. Afterwards, EDTA was added to each reaction to a final concentration of 10 mM and the samples analyzed via HPLC to quantify the remaining fragment concentrations and product yields.

The conversion results are shown in Table 8. Activity relative to SEQ ID NO: 396 was calculated as the percent conversion formed by the variant over percent conversion of SEQ ID NO: 396.

TABLE 8
RNA ligase activity for total A conversion, antisense A strand conversion,
and sense A strand conversion relative to SEQ ID NO: 396

| | Amino Acid | | Pos Control | Pos Control |
|---|---|---|---|---|
| SEQ ID | Differences | Pos Control | FIOP Anti | FIOP Sense |
| NO: | (Relative to | FIOP Total A | Sense A Strand | A Strand |
| (nt/aa) | SEQ ID NO: 396) | Conversion | Conversion | Conversion |
| 427/428 | A102T/G119E | ++ | | ++ |
| 429/430 | D75T/A102T | ++ | ++ | ++ |
| 431/432 | D75T/A102T/W117M | ++ | | +++ |
| 433/434 | D75T/G119E | | | ++ |
| 435/436 | D75T/T136A/G296W | +++ | ++ | +++ |
| 437/438 | D75T/V327W | + | + | ++ |
| 439/440 | D75T/W117K/G119E | ++ | ++ | ++ |
| 441/442 | G119E | + | | ++ |
| 443/444 | G119E/G296W | + | | ++ |
| 445/446 | K14T/A102T/G119E | ++ | + | +++ |
| 447/448 | K14T/A102T/G296W | ++ | ++ | ++ |
| 449/450 | K14T/D75T | +++ | ++ | +++ |
| 451/452 | K14T/D75T/G119E | ++ | | ++ |
| 453/454 | K14T/D75T/G119E/V327W | ++ | | +++ |
| 455/456 | K14T/D75T/W117M/G119E | ++ | | +++ |
| 457/458 | K14T/D75T/W117M/G119E/G296W | ++ | | +++ |
| 459/460 | K14T/G119E | ++ | | +++ |
| 461/462 | K14T/G296W | + | | ++ |
| 463/464 | K14T/K30H/Y270A | ++ | + | ++ |
| 465/466 | K14T/S25G | +++ | ++ | +++ |
| 467/468 | K14T/W117K | + | ++ | |
| 469/470 | K14T/W117K/G119E | ++ | ++ | + |
| 471/472 | K14T/W117M/G119E | ++ | | +++ |
| 473/474 | K14T/W117M/G119E/G296W | ++ | | +++ |
| 475/476 | K14T/W117M/G296W/V327W | + | | ++ |
| 477/478 | K26V/D75T/V327W | ++ | ++ | ++ |
| 479/480 | S25L/D75T | ++ | ++ | +++ |
| 481/482 | S25W/K30H/A102T | + | | + |
| 483/484 | W117M | | | + |
| 485/486 | W117M/G119E | ++ | | +++ |
| 487/488 | W117M/G119E/G296W | ++ | | +++ |

Levels of increased activity were determined relative to the reference polypeptide of SEQ ID NO: 396 and defined as follows: “+” >1, “++” >1.2, “+++” >2

### Example 11

### Improvements Over SEQ ID NO: 436 in RNA Ligase 2 Activity

The engineered RNA ligase of SEQ ID NO: 436 was selected as the parent enzyme after screening variants as described in Example 9 above. Libraries of engineered genes were produced using known techniques (e.g., saturation mutagenesis, recombination of previously identified beneficial mutations, etc.). The polypeptides encoded by each gene were produced in HTP as described in Example 1 and the cell paste was generated as described in Example 2.

To prepare cells for lysis, 600 μL of 0.1 mg/mL lysozyme in 50 mM TRIS-HCl buffer at pH 7.5 was added to the cell paste of each sample. After thorough resuspension, the cells were incubated at 43° C. for 1 hour in a PCR thermocycler. The samples were then centrifuged for 10 minutes at 4,000 rpm and 4° C. and the clarified supernatants were used in subsequent biocatalytic reactions.

To screen the enzyme variants, lysate 2% (v/v) final concentration transferred into 96-well shallow well plates containing 20 g/L of the shortmer mix A (Table 2.1) and 26.6% (v/v) reaction buffer. The reactions were incubated for 4 hours at 33° C. in a Multitron (Infors) with shaking at 700 rpm. Afterwards, EDTA was added to each reaction to a final concentration of 10 mM and the samples analyzed via HPLC to quantify the remaining fragment concentrations and product yields.

The conversion results are shown in Table 9. Activity relative to SEQ ID NO: 436 was calculated as the percent conversion formed by the variant over percent conversion of SEQ ID NO: 436.

TABLE 9
RNA ligase activity for total A conversion,
antisense A strand conversion, and sense A
strand conversion relative to SEQ ID NO: 436

| | | | Pos Control | |
|---|---|---|---|---|
| | Amino Acid | | FIOP Anti | Pos Control |
| SEQ | Differences | Pos Control | Sense A | FIOP Sense |
| ID NO: | (Relative to | FIOP Total A | Strand | A Strand |
| (nt/aa) | SEQ ID NO: 436) | Conversion | Conversion | Conversion |
| 489/490 | D332R | + | + | ++ |
| 491/492 | F156C | ++ | ++ | ++ |
| 493/494 | N177L | ++ | ++ | ++ |
| 495/496 | V333A | ++ | ++ | ++ |
| 497/498 | F96A | ++ | ++ | ++ |
| 499/500 | G11P | ++ | ++ | ++ |
| 501/502 | F156Y | ++ | ++ | ++ |
| 503/504 | T189L | ++ | ++ | ++ |
| 505/506 | N177I | ++ | ++ | ++ |
| 507/508 | V327I | ++ | ++ | ++ |
| 509/510 | V333T | ++ | ++ | ++ |
| 511/512 | T189V | ++ | ++ | ++ |
| 513/514 | S25K | ++ | ++ | ++ |
| 515/516 | A73T | ++ | ++ | ++ |
| 517/518 | V333S | ++ | ++ | ++ |
| 519/520 | Y98S | ++ | ++ | ++ |
| 521/522 | E335L | ++ | | ++ |
| 523/524 | V327R | ++ | ++ | |
| 525/526 | K95E | ++ | ++ | ++ |
| 527/528 | A188S | + | + | + |
| 529/530 | Y98E | ++ | ++ | ++ |
| 531/532 | F52L | ++ | + | ++ |
| 533/534 | E335K | ++ | ++ | ++ |

Levels of increased activity were determined relative to the reference polypeptide of SEQ ID NO: 436 and defined as follows: “+” >1, “++” >1.2

### Example 12

### Improvements Over SEQ ID NO: 520 in RNA Ligase 2 Activity

The engineered RNA ligase of SEQ ID NO: 520 was selected as the parent enzyme after screening variants as described in Example 11 above. Libraries of engineered genes were produced using known techniques (e.g., saturation mutagenesis, recombination of previously identified beneficial mutations, etc.). The polypeptides encoded by each gene were produced in HTP as described in Example 1 and the cell paste was generated as described in Example 2.

To prepare cells for lysis, 600 μL of 0.1 mg/mL lysozyme in 50 mM TRIS-HCl buffer at pH 7.5 was added to the cell paste of each sample. After thorough resuspension, the cells were incubated at 43° C. for 1 hour in a PCR thermocycler. The samples were then centrifuged for 10 minutes at 4,000 rpm and 4° C. and the clarified supernatants were used in subsequent biocatalytic reactions.

To screen the enzyme variants, lysate 1% (v/v) final concentration transferred into 96-well shallow well plates containing 20 g/L of the shortmer mix A (Table 2.1) and 26.6% (v/v) reaction buffer. The reactions were incubated for 4 hours at 33° C. in a Multitron (Infors) with shaking at 700 rpm. Afterwards, EDTA was added to each reaction to a final concentration of 10 mM and the samples analyzed via HPLC to quantify the remaining fragment concentrations and product yields.

The conversion results are shown in Table 10. Activity relative to SEQ ID NO: 520 was calculated as the percent conversion formed by the variant over percent conversion of SEQ ID NO: 520.

TABLE 10
RNA ligase activity for total A conversion, antisense A strand conversion,
and sense A strand conversion relative to SEQ ID NO: 520

| | Amino Acid | | Pos Control | Pos Control |
|---|---|---|---|---|
| | Differences | Pos Control | FIOP Anti | FIOP Sense |
| SEQ ID NO: | (Relative to | FIOP Total A | Sense A Strand | A Strand |
| (nt/aa) | SEQ ID NO: 520) | Conversion | Conversion | Conversion |
| 535/536 | F96S/S98Y/F156C/T189V/E335K | ++++ | ++++ | ++++ |
| 537/538 | A73T/F96S/S98E/T189L/E335K | ++++ | ++++ | ++++ |
| 539/540 | A73T/N177L/T189V | +++ | ++++ | +++ |
| 541/542 | A73T/S98E/N177L/T189V/E335K | +++ | +++ | ++++ |
| 543/544 | S25A/A73T/K95E/F96A/S98E/T189V/ | +++ | +++ | +++ |
| | V327I | | | |
| 545/546 | S25K/S98Y/T189V | +++ | +++ | +++ |
| 547/548 | A73T/K95E/F96S/F156Y/N177L | +++ | +++ | +++ |
| 549/550 | A73T/E335K | +++ | ++ | +++ |
| 551/552 | A73T/F96S/S98Y/F156C/E335K | +++ | ++ | +++ |
| 553/554 | A73T/N177L/T189V/V333T | ++ | ++ | ++ |

Levels of increased activity were determined relative to the reference polypeptide of SEQ ID NO: 520 and defined as follows: “+” >1, “++” >1.2, “+++” >2, “++++” >4

### Example 13

### Improvements Over SEQ ID NO: 552 in RNA Ligase 2 Activity

SEQ ID NO: 552 was selected as the parent enzyme after screening variants as described in Example 12 above. Libraries of engineered genes were produced using well established techniques (e.g., saturation mutagenesis, recombination of previously identified beneficial mutations). The polypeptides encoded by each gene were produced in HTP as described in Example 1 and the cell paste was generated as described in Example 2.

To prepare cells for lysis, 600 μL of 0.1 mg/mL lysozyme in 50 mM Tris-HCl buffer at pH 7.5 was added to the cell paste of each sample. After thorough resuspension, the cells were incubated at 43° C. for 1 hour in a PCR thermocycler. The samples were then centrifuged for 10 minutes at 4,000 rpm and 4° C. and the clarified supernatants were used in subsequent biocatalytic reactions.

The enzyme variants were screened with three shortmer mixes A, B, and C (Table 2.1, Table 11.1, Table 11.2). Screening with shortmer mix A was performed using final lysate concentration of 1% (v/v). Screening with shortmer mix B and shortmer mix C was performed using final lysate concentration of 2% (v/v). To screen the enzyme variants, lysate concentration indicated above was transferred into 96-well shallow well plates containing 20 g/L of a testing shortmer mix and 26.6% (v/v) reaction buffer. The reactions were incubated for 4 hours at 33° C. in a Multitron (Infors) with shaking at 700 rpm. Afterwards, EDTA was added to each reaction to a final concentration of 10 mM and the samples analyzed via HPLC to quantify the remaining fragment concentrations and product yields.

TABLE 11.1

| Oligo No. | Shortmer B mix components |
|---|---|
| 5 | U*-Cf*-A*-Cf-U-Gf-A-G |
| 6 | 5′-p-A-A-U-Af-C-Uf-G-Uf-C-Cf-C-Gf*-U |
| 7 | Tri-NAG*-invAb*-A-C-G-G-G |
| 8 | 5′-p-A-C-A-Gf-Uf-Af-U-U-C-U-C-A-G-U-I- |
| | A*-invAB |

In the above description of oligonucleotides, “-” represents a phosphate linkage; “*-” represents a phosphorothioate linkage; nucleotide N (e.g., A, G, U, C) represents a 2′-methoxy (2′-O-methyl) modified nucleotide; nucleotide I represents a 2′-methoxy (2′-O-methyl) modified inosine; (Nf represents a 2′-fluoro modified nucleotide; Tri-NAG represents a tri-antennary N-Acetylgalactosamine (GalNAc) ligand; invAb represents an inverted abasic residue (inverted abasic deoxyribonucleotide); and 5′-p represents 5′-phosphate. Further information regarding the full oligonucleotide construct can be found, for example, in U.S. Pat. No. 10,597,657 to Arrowhead Pharmaceuticals, Inc., incorporated by reference herein.

Oligonucleotide 5 base pairs with oligonucleotide 8, and oligonucleotide 6 base pairs with oligonucleotides 7 and 8. Oligonucleotides 5-8 when hybridized form a nick between the 3′-end of oligonucleotide 5 and 5-end of oligonucleotide 6, and a nick between the 3′-end of oligonucleotide 7 and the 5′-end of oligonucleotide 8. Ligation of oligonucleotides 5 and 6 results in formation of the anti-sense B strand, and ligation of oligonucleotides 7 and 8 results in formation of the sense B strand.

TABLE 11.2

| Oligo No. | Shortmer C mix components |
|---|---|
| 9 | cPrP-U*-Gf*-A*-U-G-U-U-U |
| 10 | 5′-p-G-A-Gf-C-Af-C-Cf-U-A-C-U*-C |
| 11 | Tri-SM-G*A-G-U-A-G-Gf |
| 12 | 5′-p-U-Gf-C-Uf-C-A-A-A-A-C-A-U-C-A*- |
| | invAb |

In the above description of oligonucleotides, “-” represents a phosphate linkage; “*-” represents a phosphorothioate linkage; nucleotide N (e.g., A, G, U, C) represents a 2′-methoxy (2′-O-methyl) modified nucleotide; Nf represents a 2′-fluoro modified nucleotide; Tri-SM represents a tri-antennary small molecule ligand having affinity for integrin alpha-v-beta-6; cPrP represents cyclopropyl phosphonate invAb represents an inverted abasic residue (inverted abasic deoxyribonucleotide); and 5′-p represents 5′-phosphate. Further information regarding the full oligonucleotide construct can be found, for example, in International Patent Application Publication No. WO 2022/216920 to Arrowhead Pharmaceuticals, Inc., incorporated by reference herein.

Oligonucleotide 9 base pairs with oligonucleotide 12, and oligonucleotide 10 base pairs with oligonucleotides 11 and 12. Oligonucleotides 9-12 when hybridized form a nick between the 3′-end of oligonucleotide 9 and 5-end of oligonucleotide 10, and a nick between the 3′-end of oligonucleotide 11 and the 5′-end of oligonucleotide 12. Ligation of oligonucleotides 9 and 10 results in formation of the anti sense C strand, and ligation of oligonucleotides 11 and 12 results in formation of the sense C strand.

Percent conversions were calculated as in the following using analytical method in Example 15 and the results are shown in Tables 11.3 to 11.4:

$$Antisense B or C strand conversion : \% conversion = \frac{Area under the curve for anti sense B or C strand product}{Total area under the curve for all products and substrates} * 100 \%$$

$$Sense B or C strand conversion : \% conversion = \frac{Area under the curve for sense B or C strand product}{Total area under the curve for all products and substrates} * 100 \%$$

$$Total B conversion : Sum of antisense B or C strand conversion and sense B or C strand conversion .$$

The conversion results are shown in Table 11.3 and 11.4. Activity relative to SEQ ID NO: 552 was calculated as the percent conversion formed by the variant over percent conversion of SEQ ID NO: 552.

TABLE 11.3
RNA ligase activity for total A conversion,
antisense A strand conversion, and sense A
strand conversion relative to SEQ ID NO: 552

| | | | Pos Control | Pos Control |
|---|---|---|---|---|
| | Amino Acid | Pos Control | FIOP Anti | FIOP Total |
| SEQ | Differences | FIOP Sense | Sense | Conversion |
| ID NO: | (Relative to | A Strand | A Strand | relative to SEQ |
| (nt/aa) | SEQ ID NO: 552) | Conversion | Conversion | ID NO: 552 |
| 555/556 | E292Y | ++ | + | ++ |
| 557/558 | Y147L | ++ | + | + |
| 559/560 | P255S | ++ | ++ | ++ |
| 561/562 | M295R | ++ | + | ++ |
| 563/564 | T75L | + | + | + |

Levels of increased activity were determined for RNA ligase 2 activity relative to SEQ ID NO: 552 and are defined as follows: “+” >1.2, “++” >2.0.

TABLE 11.4
RNA ligase 2 activity relative to SEQ ID NO: 552

| | | | | Set A Pos | | | Set B Pos | | | Set C Pos |
|---|---|---|---|---|---|---|---|---|---|---|
| | Amino Acid | Set A Pos | Set A Pos | Control | Set B Pos | Set B Pos | Control | Set C Pos | Set C Pos | Control |
| | Differences | Control | Control | FIOP | Control | Control | FIOP | Control | Control | FIOP |
| SEQ ID NO: | (Relative to | FIOP SS | FIOP AS | Total | FIOP SS | FIOP AS | Total | FIOP SS | FIOP AS | Total |
| (nt/aa) | SEQ ID NO: 552) | Conver.¹ | Conver.¹ | Conver.¹ | Conver.¹ | Conver.¹ | Conver.¹ | Conver.¹ | Conver.¹ | Conver.¹ |
| 565/566 | Q300E | ++ | ++ | ++ | | | | + | + | + |
| 567/568 | V333E | + | ++ | ++ | ++ | + | + | + | + | + |
| 569/570 | C200S | ++ | ++ | ++ | + | + | + | + | + | + |
| 571/572 | K289S | | + | + | ++ | | + | + | + | + |
| 573/574 | K95V | + | ++ | + | + | + | + | + | + | + |
| 575/576 | K289A | + | ++ | ++ | ++ | | + | ++ | ++ | ++ |
| 577/578 | K289T | + | ++ | + | ++ | + | + | ++ | + | ++ |
| 579/580 | T168A | + | ++ | + | | | | + | + | + |
| 581/582 | K289H | + | ++ | ++ | ++ | | + | ++ | + | + |
| 583/584 | C200T | ++ | ++ | ++ | + | + | + | + | + | + |

¹Conversion relative to SEQ ID NO: 552.
Levels of increased activity were determined for RNA ligase 2 activity relative to SEQ ID NO: 552 and are defined as follows: “+” >1.0, “++” >1.2.

### Example 14

### Improvements Over SEQ ID NO: 582 in RNA Ligase 2 Activity

The RNA ligase of SEQ ID NO: 582 was selected as the parent enzyme after screening variants as described in Example 13 above. Libraries of engineered genes were produced using well established techniques (e.g., saturation mutagenesis, recombination of previously identified beneficial mutations). The polypeptides encoded by each gene were produced in HTP as described in Example 1 and the cell paste was generated as described in Example 2.

To prepare cells for lysis, 600 μL of 0.1 mg/mL lysozyme in 50 mM Tris-HCl buffer at pH 7.5 was added to the cell paste of each sample. After thorough resuspension, the cells were incubated at 43° C. for 1 hour in a PCR thermocycler. The samples were then centrifuged for 10 minutes at 4,000 rpm and 4° C. and the clarified supernatants were used in subsequent biocatalytic reactions.

The enzyme variants were screened with three shortmer mixes A, B, and C (Tables 2.1, 11.1 and 11.2). Screening with shortmer mix A and shortmer mix C was performed using final lysate concentration of 0.625% (v/v). Screening with shortmer mix B was performed using final lysate concentration of 2% (v/v). To screen the enzyme variants, lysate concentration indicated above was transferred into 96-well shallow well plates containing 20 g/L of a testing shortmer mix and 26.6% (v/v) reaction buffer. The reactions were incubated for 4 hours at 33° C. in a Multitron (Infors) with shaking at 700 rpm. Afterwards, EDTA was added to each reaction to a final concentration of 10 mM and the samples analyzed via HPLC to quantify the remaining fragment concentrations and product yields.

Percent conversions were calculated as described in Example 13 the following using analytical method in Example 15.

The conversion results are shown in Table 12. Activity relative to SEQ ID NO: 582 was calculated as the percent conversion formed by the variant over percent conversion of SEQ ID NO: 582.

TABLE 12
RNA ligase activity relative to SEQ ID NO: 582

| | | | | Set A Pos | | | Set B Pos | | | Set C Pos |
|---|---|---|---|---|---|---|---|---|---|---|
| | Amino Acid | Set A Pos | Set A Pos | Control | Set B Pos | Set B Pos | Control | Set C Pos | Set C Pos | Control |
| SEQ | Differences | Control | Control | FIOP | Control | Control | FIOP | Control | Control | FIOP |
| ID NO: | (Relative to | FIOP SS | FIOP AS | Total | FIOP SS | FIOP AS | Total | FIOP SS | FIOP AS | Total |
| (nt/aa) | SEQ ID NO: 582) | Conver.¹ | Conver.¹ | Conver.¹ | Conver.¹ | Conver.¹ | Conver.¹ | Conver.¹ | Conver.¹ | Conver.¹ |
| 585/586 | K95V/C156Y/A337G | +++ | +++ | +++ | ++ | +++ | ++ | +++ | +++ | +++ |
| 587/588 | T189V/C200T/V271I | ++ | ++ | ++ | + | + | + | + | + | + |
| 589/590 | C200S/V271I/H289A/A337G | ++ | ++ | ++ | + | ++ | + | + | + | + |
| 591/592 | C200T/V271I/H289A | +++ | ++ | +++ | ++ | ++ | ++ | ++ | ++ | ++ |
| 593/594 | T189V/C200S | ++ | ++ | ++ | + | + | + | + | + | + |
| 595/596 | C156Y/C200S/H289T | ++ | ++ | ++ | + | ++ | + | ++ | ++ | ++ |
| 597/598 | C156F/T168A/T189V/V271I/ | ++ | + | ++ | + | + | + | + | + | + |
| | Q300E | | | | | | | | | |
| 599/600 | C156Y/T189V/C200T/H289A | +++ | ++ | +++ | ++ | ++ | ++ | ++ | ++ | ++ |
| 601/602 | C156F/T168A/H289K | ++ | ++ | ++ | + | | + | + | + | + |
| 603/604 | K95V/C156Y/V271I/H289K | ++ | ++ | ++ | + | | + | + | + | + |
| 605/606 | C156F/V333E | +++ | +++ | +++ | + | + | + | ++ | ++ | ++ |
| 607/608 | K95V/C200T/H289K/A337G | ++ | ++ | ++ | ++ | + | + | ++ | + | + |
| 609/610 | T168A/C200S/H289A | ++ | ++ | ++ | + | + | + | + | + | + |
| 611/612 | T189V/C200T/H289K | ++ | ++ | ++ | ++ | | + | + | + | + |
| 613/614 | V333E/A337G | +++ | ++ | ++ | + | + | + | + | + | + |
| 615/616 | V103I/C156F/V271I | + | + | + | + | + | + | + | + | + |
| 617/618 | K95V/C156F/C200S/Q300E/ | ++ | ++ | ++ | + | + | + | + | + | + |
| | V333E | | | | | | | | | |
| 619/620 | C156F/V271I/H289T/V333E | +++ | ++ | +++ | + | ++ | + | ++ | ++ | ++ |
| 621/622 | K95V/C156F/V271I | +++ | +++ | +++ | ++ | ++ | ++ | ++ | ++ | ++ |
| 623/624 | K95V/T189V/H289K | + | ++ | ++ | + | | + | + | + | + |
| 625/626 | K95V/C156Y/T168A/T189V/ | +++ | +++ | +++ | ++ | ++ | ++ | ++ | ++ | ++ |
| | V271I/H289T/Q300E | | | | | | | | | |
| 627/628 | T189V/V271I/H289A | ++ | + | ++ | + | + | + | + | + | + |
| 629/630 | K95V/T168A/V271I/Q300E | ++ | ++ | ++ | + | + | + | + | + | + |
| 631/632 | C156Y/T189V/H289A/A337G | ++ | ++ | ++ | ++ | ++ | ++ | ++ | ++ | ++ |
| 633/634 | C200S/V271I | ++ | ++ | ++ | + | + | + | + | + | + |
| 635/636 | C156F/T189V/V333E | ++ | ++ | ++ | + | + | + | + | + | + |
| 637/638 | T189V/H289T | +++ | ++ | ++ | ++ | ++ | ++ | ++ | ++ | ++ |
| 639/640 | C156Y/T189V/C200T/Q300E/ | ++ | ++ | ++ | + | + | + | + | + | + |
| | V333E/A337G | | | | | | | | | |
| 641/642 | C156Y/C200T/H289A | ++ | ++ | ++ | + | + | + | + | + | + |
| 643/644 | C156F/T189V/C200S/V271I/ | + | ++ | ++ | + | + | + | + | + | + |
| | H289K/A337G | | | | | | | | | |
| 645/646 | C156Y/C200S/V271I/H289A/ | +++ | ++ | ++ | + | ++ | + | + | + | + |
| | V333E/A337G | | | | | | | | | |
| 647/648 | T168A/V271I/Q300E | ++ | + | ++ | + | + | + | + | + | + |
| 649/650 | H289T/A337G | ++ | ++ | ++ | + | + | + | + | + | + |
| 651/652 | C156Y/T168A/A337G | ++ | + | + | + | + | + | + | + | + |
| 653/654 | C156F/T168A/T189V/H289T | + | + | + | + | + | + | + | + | + |
| 655/656 | C156Y/V271I/H289T | + | + | + | + | + | + | + | + | + |
| 657/658 | T189V/Q300E | ++ | + | + | + | + | + | + | + | + |
| 659/660 | C156Y/H289T/Q300E/A337G | ++ | + | + | + | + | + | + | + | + |
| 661/662 | T168A/T189V/H289T | + | + | + | + | + | + | + | + | + |
| 663/664 | K95V/C156F/T168A/Q300E | ++ | ++ | ++ | + | ++ | ++ | ++ | ++ | ++ |
| 665/666 | C156Y/C200S/Q300E/A337G | + | + | + | + | + | + | + | + | + |
| 667/668 | H289A/V333E/A337G | + | + | + | + | + | + | + | + | + |
| 669/670 | K95V/T189V/C200T/Q300E/ | + | + | + | + | + | + | + | + | + |
| | A337G | | | | | | | | | |
| 671/672 | K95V/C156F/T189V/C200T/ | ++ | ++ | ++ | ++ | +++ | ++ | ++ | ++ | ++ |
| | V271I/V333E | | | | | | | | | |
| 673/674 | C200S/V271I/H289A/Q300E | + | + | + | + | + | + | + | + | + |
| 675/676 | H289A/V333E | ++ | ++ | ++ | + | + | + | + | + | + |
| 677/678 | T189V/V271I/Q300E | + | + | + | + | + | + | + | + | + |
| 679/680 | K95V/Q300E | + | + | + | + | + | + | + | + | + |
| 681/682 | K95V/T189V/V333E/A337G | + | + | + | + | + | + | + | + | + |
| 683/684 | K95V/T189V/V271I/Q300E | + | + | + | + | + | + | + | + | + |
| 685/686 | K95V/T189V/C200S/H289A | +++ | ++ | +++ | ++ | ++ | ++ | ++ | ++ | ++ |
| 687/688 | T168A | ++ | ++ | ++ | + | ++ | + | ++ | ++ | ++ |
| 689/690 | C156Y/C200S/V271I/H289K/ | ++ | ++ | ++ | + | ++ | + | ++ | ++ | ++ |
| | A337G | | | | | | | | | |
| 691/692 | K95V/T168A/H289T | ++ | + | ++ | + | + | + | + | + | + |
| 693/694 | C156F/H289A | ++ | ++ | ++ | + | ++ | + | ++ | ++ | ++ |
| 695/696 | K95V/C156Y/T189V/V271I/ | + | + | + | + | + | + | + | + | + |
| | H289K | | | | | | | | | |
| 697/698 | C156F/T168A/T189V/C200S/ | +++ | +++ | +++ | + | ++ | + | ++ | ++ | ++ |
| | H289A/V333E | | | | | | | | | |
| 699/700 | C156F/Q300E/V333E/A337G | ++ | ++ | ++ | + | ++ | + | ++ | + | ++ |
| 701/702 | K95V/C156F/T189V/C200S/ | + | + | + | + | ++ | + | + | + | + |
| | V271I/A337G | | | | | | | | | |
| 703/704 | C156F/T189V/C200T/H289K | ++ | ++ | ++ | + | + | + | ++ | + | ++ |
| 705/706 | C156Y/H289T | +++ | ++ | ++ | + | ++ | + | ++ | ++ | ++ |
| 707/708 | V271I | ++ | ++ | ++ | + | ++ | + | ++ | ++ | ++ |
| 709/710 | C156Y/V271I | ++ | ++ | ++ | + | ++ | + | ++ | ++ | ++ |
| 711/712 | C156F/V271I/Q300E/V333E | +++ | ++ | +++ | + | ++ | + | ++ | + | ++ |
| 713/714 | K95V/T168A/C200S | ++ | ++ | ++ | + | + | + | + | + | + |
| 715/716 | T189V/C200S/H289A | + | + | + | + | + | + | + | + | + |
| 717/718 | T168A/V271I | ++ | ++ | ++ | + | ++ | ++ | ++ | ++ | ++ |
| 719/720 | K95V/C200T/V333E | +++ | ++ | +++ | + | ++ | + | ++ | ++ | |
| 721/722 | T189V/C200S/V271I/H289A/ | +++ | +++ | +++ | ++ | +++ | ++ | +++ | ++ | +++ |
| | V333E | | | | | | | | | |
| 723/724 | K95V/T168A/T189V/V333E/ | ++ | ++ | ++ | + | + | + | + | + | + |
| | A337G | | | | | | | | | |
| 725/726 | T189V | ++ | ++ | ++ | + | + | + | ++ | + | + |
| 727/728 | T189V/V333E/A337G | +++ | ++ | ++ | + | ++ | + | ++ | ++ | ++ |
| 729/730 | C156F/T189V/V271I/H289A/ | +++ | ++ | +++ | + | ++ | + | ++ | ++ | ++ |
| | V333E | | | | | | | | | |
| 731/732 | H289A | ++ | ++ | ++ | + | + | + | + | + | + |
| 733/734 | K95V/C156F/C200T/V271I/ | +++ | +++ | +++ | ++ | +++ | ++ | +++ | ++ | ++ |
| | H289K | | | | | | | | | |
| 735/736 | H289A/Q300E/V333E | +++ | +++ | +++ | + | ++ | + | ++ | ++ | ++ |
| 737/738 | K95V/T189V/H289A/A337G | +++ | ++ | ++ | + | ++ | ++ | ++ | ++ | ++ |
| 739/740 | K95V/C156F/T168A/T189V/ | +++ | +++ | +++ | ++ | +++ | ++ | +++ | +++ | +++ |
| | H289A | | | | | | | | | |
| 741/742 | V271I/H289T | +++ | ++ | ++ | + | ++ | ++ | ++ | ++ | ++ |
| 743/744 | V333E | +++ | +++ | +++ | + | + | + | ++ | ++ | ++ |
| 745/746 | T168A/A337G | ++ | ++ | ++ | + | + | + | + | + | + |
| 747/748 | C156Y/H289A | ++ | ++ | ++ | + | ++ | + | + | + | + |
| 749/750 | C156Y/T189V/V271I/H289K | + | ++ | ++ | + | + | + | + | + | + |
| 751/752 | K95V/C156F/T189V/A337G | ++ | ++ | ++ | ++ | ++ | ++ | ++ | ++ | ++ |
| 753/754 | T189V/H289K | ++ | ++ | ++ | ++ | + | + | ++ | + | ++ |
| 755/756 | T189V/H289A | +++ | +++ | +++ | ++ | ++ | ++ | ++ | ++ | ++ |
| 757/758 | C156Y/T189V/V271I/Q300E | ++ | + | ++ | + | ++ | + | ++ | + | + |
| 759/760 | C156F/T168A/H289A/V333E | +++ | +++ | +++ | + | ++ | + | ++ | ++ | ++ |
| 761/762 | T168A/T189V/H289K/V333E | ++ | ++ | ++ | ++ | ++ | ++ | + | + | + |
| 763/764 | K95V/V333E | ++ | ++ | ++ | + | ++ | + | + | + | + |
| 765/766 | V333E/S343P | ++ | ++ | ++ | ++ | ++ | ++ | ++ | ++ | ++ |
| 767/768 | C156F/T189V/H289A | ++ | + | ++ | + | ++ | ++ | + | + | + |
| 769/770 | K95V/H289A/Q300E | ++ | + | ++ | + | + | + | + | + | + |
| 771/772 | K95V/C156Y/T189V/C200S/ | ++ | + | ++ | + | + | + | + | + | + |
| | Q300E/V333E/A337G | | | | | | | | | |
| 773/774 | T189V/C200T/V271I/H289K/ | + | + | + | ++ | ++ | ++ | + | + | + |
| | A337G | | | | | | | | | |
| 775/776 | K95V/C156Y/T168A/T189V/ | + | ++ | ++ | ++ | ++ | ++ | ++ | + | + |
| | H289K/A337G | | | | | | | | | |
| 777/778 | C156F/H289K | + | ++ | ++ | ++ | +++ | ++ | ++ | + | ++ |
| 779/780 | K95V/H289A | + | + | + | + | + | + | + | + | + |
| 781/782 | C156Y/T168A/H289A | +++ | ++ | +++ | ++ | +++ | ++ | ++ | ++ | ++ |
| 783/784 | C156F/H289K/V333E/A337G | + | ++ | + | + | + | + | + | + | + |
| 785/786 | C156F/H289K/A337G | + | ++ | + | ++ | ++ | ++ | + | + | + |
| 787/788 | H289A/A337G | ++ | ++ | ++ | ++ | ++ | ++ | ++ | ++ | ++ |
| 789/790 | A337G | + | + | + | + | + | + | + | + | + |
| 791/792 | T168A/H289A | ++ | ++ | ++ | ++ | ++ | ++ | ++ | ++ | ++ |
| 793/794 | K95V/C156F/Q300E | ++ | ++ | ++ | ++ | +++ | ++ | ++ | + | + |
| 795/796 | K95V/C156Y/H289K | + | + | + | + | + | + | + | + | + |
| 797/798 | K95V/T189V/V271I/V333E/ | ++ | + | + | + | ++ | + | + | + | + |
| | A337G | | | | | | | | | |
| 799/800 | K95V/T168A/Q300E | ++ | + | ++ | + | ++ | + | + | + | + |
| 801/802 | K95V/C156Y/T168A/V271I/ | ++ | + | ++ | + | ++ | + | + | + | + |
| | V333E/A337G | | | | | | | | | |
| 803/804 | K95V/T189V/H289A/V333E/ | ++ | ++ | ++ | + | ++ | + | + | + | + |
| | A337G | | | | | | | | | |
| 805/806 | C156F/T168A/C200S/H289A/ | ++ | | + | + | ++ | ++ | + | + | + |
| | L334S | | | | | | | | | |
| 807/808 | K95V/C156F/T168A/T189V/ | + | | + | ++ | +++ | ++ | + | + | + |
| | V271I/H289T | | | | | | | | | |
| 809/810 | C156Y/T189V/C200T/H289T/ | +++ | ++ | ++ | ++ | +++ | ++ | ++ | ++ | ++ |
| | A337G | | | | | | | | | |
| 811/812 | C156F/V271I | ++ | + | + | + | ++ | ++ | + | + | + |
| 813/814 | H289K/Q300E/V333E | ++ | ++ | ++ | ++ | ++ | ++ | ++ | + | ++ |
| 815/816 | K95V/C156Y/V271I | +++ | ++ | ++ | ++ | +++ | ++ | +++ | ++ | ++ |
| 817/818 | C200S/H289A | ++ | + | ++ | + | ++ | + | + | + | + |
| 819/820 | C200S/H289K/V333E/A337G | + | ++ | ++ | + | + | + | + | + | + |
| 821/822 | K95V/T168A/T189V/C200T/ | ++ | + | + | + | ++ | + | + | + | + |
| | H289A | | | | | | | | | |
| 823/824 | C156Y/V271I/H289T/V333E | +++ | ++ | +++ | ++ | +++ | ++ | ++ | ++ | ++ |
| 825/826 | T168A/C200S | + | + | + | + | ++ | + | + | + | + |
| 827/828 | C156F/T168A/V271I/H289A | ++ | + | ++ | ++ | ++ | ++ | ++ | ++ | ++ |
| 829/830 | T189L/V206R/H289S | + | + | + | + | + | + | + | + | + |
| 831/832 | C156F/C200S/H289S/V333E/ | ++ | + | ++ | + | + | + | + | + | + |
| | K335E | | | | | | | | | |
| 833/834 | C156F/C200S/V333E | ++ | ++ | ++ | ++ | ++ | ++ | ++ | ++ | ++ |
| 835-836 | T189V/C200S/H289K/V333E | ++ | ++ | ++ | + | + | + | ++ | + | + |
| 837/838 | T189L/H289K/K335E | ++ | ++ | ++ | + | + | + | + | + | + |
| 839/840 | T189V/V254G/H289S/V333E | ++ | ++ | ++ | + | + | + | + | + | + |
| 841/842 | T189L/H289S | ++ | ++ | ++ | + | + | + | + | + | + |
| 843/844 | G167A/T189L/V206R/H289S/ | ++ | + | ++ | + | ++ | + | + | + | + |
| | V333E/K335E | | | | | | | | | |
| 845/846 | C156F/C200S/H289S/V333E | + | + | + | + | + | + | + | + | + |
| 847/848 | T189L/C200S/H289S | + | + | + | + | + | + | + | + | + |
| 849/850 | C156Y/C200S/H289K/K335E | + | + | + | + | + | + | + | + | + |
| 851/852 | C156F/T189L/C200S/H289K | + | ++ | + | ++ | + | + | + | + | + |
| 853/854 | C156Y/T189L/H289K/K335E | ++ | + | ++ | + | + | + | + | + | + |
| 855/856 | C156Y/C200S/H289K/V333E | ++ | ++ | ++ | + | + | + | ++ | + | + |
| 857/858 | K335E | ++ | + | ++ | + | ++ | + | + | + | + |
| 859/860 | C156F/C200S/H289K/V333E | + | ++ | ++ | + | | + | + | + | + |
| 861/862 | C156F/G167A/T189V/H289T | ++ | ++ | ++ | + | + | + | ++ | + | + |
| 863/864 | C156Y/T189L/C200S/H289S/ | ++ | + | ++ | + | ++ | + | + | + | + |
| | K335E | | | | | | | | | |
| 865/866 | C200S/H289S/V333E/K335E | ++ | + | ++ | + | ++ | + | + | + | + |
| 867/868 | C156Y/T189V/C200S/H289T/ | ++ | ++ | ++ | + | ++ | + | ++ | + | ++ |
| | V333E | | | | | | | | | |
| 869/870 | V254G/V333E/K335E | ++ | + | ++ | + | + | + | + | + | + |
| 871/872 | T189L/V254G/V333E/K335E | ++ | + | ++ | + | ++ | + | + | + | + |
| 873/874 | H289S | ++ | ++ | ++ | ++ | ++ | ++ | ++ | ++ | ++ |
| 875/876 | T189V/H289S | ++ | ++ | ++ | + | ++ | ++ | ++ | + | ++ |
| 877/878 | T189V/K335E | ++ | + | ++ | + | + | + | + | + | + |
| 879/880 | C156F/H289T | ++ | ++ | ++ | + | + | + | + | + | + |
| 881/882 | C156F/T189V/V254G/H289S | ++ | ++ | ++ | + | ++ | + | + | + | + |
| 883/884 | C156Y/H289K/V333E | ++ | ++ | ++ | ++ | + | + | ++ | + | + |
| 885/886 | H289S/K335E | +++ | ++ | ++ | + | ++ | ++ | ++ | ++ | ++ |
| 887/888 | T189L/C200S/V254G/H289S/ | + | ++ | + | + | + | + | + | + | + |
| | V333E | | | | | | | | | |
| 889/890 | C156F/T189L/H289S/V333E | ++ | + | + | + | + | + | + | + | + |
| 891/892 | C156Y/T189V/H289S/K335E | ++ | + | + | + | + | + | + | + | + |
| 893/894 | S25A/C156Y/T189L/H289S | ++ | + | + | + | + | + | + | + | + |
| 895/896 | C156Y/T189V/V206R/H289K/ | ++ | ++ | ++ | + | + | + | + | + | + |
| | V333E/K335E | | | | | | | | | |
| 897/898 | H289S/V333E/K335E | ++ | + | ++ | + | + | + | + | + | + |
| 899/900 | T189V/C200S/H289S | + | + | + | + | + | + | + | + | + |
| 901/902 | C156F/V254G/H289K/V333E | ++ | ++ | ++ | + | + | + | + | + | + |
| 903/904 | C156Y/H289T/V333E/K335E | +++ | + | ++ | + | + | + | + | + | + |
| 905/906 | V254G/H289T | ++ | + | + | + | ++ | + | + | + | + |
| 907/908 | C200S/H289T/V333E | +++ | ++ | +++ | + | ++ | ++ | ++ | ++ | ++ |
| 909/910 | C156Y/T189V/C200S | + | + | + | + | + | + | + | + | + |
| 911/912 | C156Y/V254G/V333E/K335E | ++ | + | ++ | | + | + | + | + | + |
| 913/914 | C156F | ++ | ++ | ++ | + | ++ | + | + | + | + |
| 915/916 | V254G/H289K/K335E | + | + | + | + | + | + | + | + | + |
| 917/918 | C200S/H289T | ++ | + | ++ | + | + | + | + | + | + |
| 919/920 | C200S/H289S | ++ | + | ++ | + | + | + | + | + | + |
| 921/922 | C156Y/V206R/H289S | ++ | + | + | + | + | + | + | + | + |
| 923/924 | C156F/H289S/K335E | ++ | | + | + | ++ | + | + | + | + |
| 925/926 | V206R/H289T/V333E/K335E | +++ | + | ++ | + | + | + | + | + | + |
| 927/928 | C156F/T189V/V254G/H289K/ | + | ++ | ++ | + | + | + | + | + | + |
| | V333E | | | | | | | | | |
| 929/930 | C156F/V254G/H289T | + | + | + | + | + | + | + | + | + |
| 931/932 | C200S/H289T/K335E | ++ | + | ++ | + | ++ | + | + | + | + |
| 933/934 | C156Y/T189V/V206R/H289S | ++ | + | + | + | + | + | + | + | + |
| 935/936 | C156Y/T189V/C200S/V206R/ | ++ | ++ | ++ | + | + | + | + | + | + |
| | V254G/H289T/V333E | | | | | | | | | |
| 937/938 | C156Y/V333E | ++ | ++ | ++ | + | + | + | + | + | + |
| 939/940 | C156Y/T189L/V254G/H289T/ | ++ | ++ | ++ | + | ++ | + | + | + | + |
| | V333E | | | | | | | | | |
| 941/942 | S25A/V254G/V333E | ++ | ++ | ++ | + | + | + | + | + | + |
| 943/944 | T189L/H289S/V333E/K335E | +++ | ++ | +++ | + | ++ | + | ++ | ++ | ++ |
| 945/946 | C200S/H289K/K335E | ++ | + | + | + | + | + | + | + | + |
| 947/948 | C156Y/T189V/H289S | ++ | ++ | ++ | + | ++ | + | + | + | + |
| 949/950 | C156F/T189L/C200S/H289K/ | +++ | ++ | ++ | + | + | + | + | + | + |
| | V333E/K335E | | | | | | | | | |
| 951/952 | H289T | ++ | + | ++ | + | + | + | + | + | + |
| 953/954 | C156F/C200S/H289T/K335E | ++ | | + | + | ++ | + | + | + | + |
| 955/956 | V254G/H289S/V333E | ++ | ++ | ++ | + | ++ | + | + | + | + |
| 957/958 | C156F/C200S/V333E/K335E | ++ | + | ++ | | + | + | + | + | + |

¹Conversion relative to SEQ ID NO: 582.
Levels of increased activity were determined for RNA ligase 2 activity relative to SEQ ID NO: 582 and are defined as follows: “+” >1.2, “++” >1.8, “+++” >3.0.

### Example 15

### Analytical Detection of RNA Oligomers by HPLC-UV

Data described in Examples 4-12 was collected using the analytical method provided in Table 11. The method provided herein finds use in analyzing the variants produced using the present invention. However, it is not intended that present invention be limited to the methods described herein, as there are other suitable methods known in the art that are applicable to the analysis of the variants provided herein and/or produced using the methods provided herein.

TABLE 11
HPLC analytical method

| Instrument | Agilent 1290 - HPLC | | |
|---|---|---|---|
| Column | DNAPac PA200, 4 × 250 mm (p/n 063000) | | |
| Mobile | A: 10 mM NaHCO₃ (pH 11.3), 50 mM NaBr, 45% MeCN | | |
| Phase | B: 10 mM NaHCO₃ (pH 11.3), 650 mM NaBr, 45% MeCN | | |
| Gradient | Time (min) | % A | % B |
| | 0.0 | 70 | 30 |
| | 0.5 | 70 | 30 |
| | 3.0 | 30 | 70 |
| | 3.01 | 70 | 30 |
| | 4.0 | 70 | 30 |
| Flow Rate | 1.0 mL/min | | |
| Run Time | 4.0 min | | |
| | | Retention | |
| | Compound | time (min) | |
| Retention | ATP | 1.1 | |
| times | SS10 | 1.4 | |
| | SS14 + AS14 + AS7 | 2.6-3.0 | |
| | SS FLP | 3.1 | |
| | AS FLP | 3.65 | |

| | |
|---|---|
| Column | 50° C. |
| Temperature | |
| Injection | 10 μL |
| Volume | |
| Detection | UV 260 nm |

While the invention has been described with reference to the specific embodiments, various changes can be made and equivalents can be substituted to adapt to a particular situation, material, composition of matter, process, process step or steps, thereby achieving benefits of the invention without departing from the scope of what is claimed.

For all purposes, each and every publication and patent document cited in this disclosure is incorporated herein by reference as if each such publication or document was specifically and individually indicated to be incorporated herein by reference. Citation of publications and patent documents is not intended as an indication that any such document is pertinent prior art, nor does it constitute an admission as to its contents or date.

1. An engineered RNA ligase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of an even numbered SEQ ID NO. of SEQ ID NOs: 2-220, 224-252, and 270-958, or to a reference sequence corresponding to an even numbered SEQ ID NO. of SEQ ID NOs: 2-220, 224-252, and 270-958, wherein the amino acid sequence comprises one or more substitutions relative to the reference sequence corresponding to residues 12 to 343 of SEQ ID NO: 2, 4, 106, 218, 286, 396, 436, 520, 552, or 582, or relative to the reference sequence corresponding to SEQ ID NO: 2, 4, 106, 218, 286, 396, 436, 520, 552, or 582.

2. The engineered RNA ligase of claim 1, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of SEQ ID NO: 2, 4, 106, 218, 286, 396, 436, 520, 552, or 582, or to a reference sequence corresponding to SEQ ID NO: 2, 4, 106, 218, 286, 396, 436, 520, 552, or 582, wherein the amino acid sequence comprises one or more substitutions relative to the reference sequence corresponding to residues 12 to 343 of SEQ ID NO: 2, 4, 106, 218, 286, 396, 436, 520, 552, or 582, or relative to the reference sequence corresponding to SEQ ID NO: 2, 4, 106, 218, 286, 396, 436, 520, 552, or 582.

3. The engineered RNA ligase of claim 1, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 343 of SEQ ID NO: 2, or to the reference sequence corresponding to SEQ ID NO: 2, wherein the amino acid sequence comprises one or more substitutions relative to the reference sequence corresponding to residues 12 to 343 of SEQ ID NO: 2, or relative to the reference sequence corresponding to SEQ ID NO: 2.

4. The engineered RNA ligase of claim 1, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 343 of SEQ ID NO: 4, 106, 218, 286, 396, 436, 520, 552, or 582, or to the reference sequence corresponding to SEQ ID NO: 4, 106, 218, 286, 396, 436, 520, 552, or 582, wherein the amino acid sequence comprises one or more substitutions relative to the reference sequence corresponding to residues 12 to 343 of SEQ ID NO: 2, or relative to the reference sequence corresponding to SEQ ID NO: 2.

5. The engineered RNA ligase of claim 1, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12 to 343 of an even-numbered SEQ ID NO. of SEQ ID NOs: 4-220, 224-252, and 270-958, or to a reference sequence corresponding to an even-numbered SEQ ID NO. of SEQ ID NOs: 4-220, 224-252, and 270-958, wherein the amino acid sequence comprises one or more substitutions relative to the reference sequence corresponding to residues 12 to 343 of SEQ ID NO: 2, or relative to the reference sequence corresponding to SEQ ID NO: 2.

6. The engineered RNA ligase of claim 1, wherein the amino acid sequence of the engineered RNA ligase comprises at least a substitution at amino acid position 11, 12, 14, 16, 17, 23, 25, 26, 30, 32, 33, 38, 40, 52, 57, 60, 68, 69, 71, 73, 75, 79, 82, 83, 92, 93, 95, 96, 98, 100, 101, 102, 103, 111, 114, 117, 118, 119, 120, 135, 136, 141, 142, 145, 147, 156, 167, 168, 170, 171, 174, 177, 179, 183, 184, 185, 188, 189, 190, 191, 192, 193, 196, 200, 202, 205, 206, 207, 209, 221, 229, 254, 255, 259, 270, 271, 287, 288, 289, 292, 295, 296, 300, 307, 320, 327, 332, 333, 334, 335, 336, 337, 339, 342, or 343, or combinations thereof, wherein the amino acid positions are relative to the reference sequence corresponding to residues 12 to 343 of SEQ ID NO: 2, or relative to the reference sequence corresponding to SEQ ID NO: 2.

7. The engineered RNA ligase of claim 1, wherein the amino acid sequence of the engineered RNA ligase comprises at least a substitution or amino acid residue 11P, 12S, 14F/R/T/W, 16R, 17P, 23M, 25A/G/K/L/W, 26F/G/H/L/P/R/S/V, 30A/C/H/R, 32R, 33A/K, 38D, 40E, 52L, 57T, 60R, 68W, 69L, 71K, 73T, 75L/Q/T, 79W, 82I, 83P, 92A/E/G/M/N/S, 93A, 95E/V, 96A/S, 98D/E/P/R/S, 100R, 101G, 102T, 103I, 111L, 114F/G/K/N/P/R/S/V, 117A/C/F/G/H/I/K/L/M/R/S/T/V/W, 118G/L/Q, 119A/E/S/T, 120P, 135Q/T, 136A, 141A/C/E/T, 142H/R, 145D, 147L, 156C/Y, 167A, 168A, 170E, 171E, 174Q, 177I/L, 179F, 183V, 184L/M/P/R, 185H/S, 188S, 189L/V, 190F, 191K, 192V, 193N, 196K, 200S/T, 202W, 205R/V, 206R, 207S/T, 209E/L, 221E, 229A, 254G, 255E/S, 259V, 270A, 271I, 287A, 288A, 289A/H/S/T, 292E/Y, 295R, 296E/L/R/V/W, 300E/K, 307A, 320D/K, 327I/Q/R/W, 332R, 333A/E/S/T, 334S, 335E/H/K/L, 336S, 337G, 339K/R, 342I/T, 343P, 339K/R, or 342I/T, or combinations thereof, wherein the amino acid positions are relative to the reference sequence corresponding to residues 12 to 343 of SEQ ID NO: 2, or relative to the reference sequence corresponding to SEQ ID NO: 2.

8. The engineered RNA ligase of claim 1, wherein the amino acid sequence of the engineered RNA ligase comprises at least a substitution at amino acid position 33, 38, 71, 73, 75, 96, 98, 101, 114, 117, 136, 156, 179, 184, 191, 196, 221, 289, 292, 296, 320, 335, 336, or 339, or combinations thereof, wherein the amino acid positions are relative to the reference sequence corresponding to residues 12 to 343 of SEQ ID NO: 2, or relative to the reference sequence corresponding to SEQ ID NO: 2.

9. The engineered RNA ligase of claim 1, wherein the amino acid sequence of the engineered RNA ligase comprises at least a substitution or amino acid residue 33A/K, 38D, 71K, 73T, 75L/Q/T, 96A/S, 98D/E/P/R/S, 101G, 114F/K/N/P/R/S/V, 117A/C/F/G/H/I/K/L/M/R/S/T/V/W, 136A, 156C/Y, 179F, 184L/M/P/R, 191K, 196K, 221E, 289A/H/S/T, 292E/Y, 296E/L/R/V/W, 320D/K, 335E/H/K/L, 336S, or 339K/R, or combinations thereof, wherein the amino acid positions are relative to the reference sequence corresponding to residues 12 to 343 of SEQ ID NO: 2, or relative to the reference sequence corresponding to SEQ ID NO: 2.

10-18. (canceled)

19. The engineered RNA ligase of claim 1, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of SEQ ID NO: 4, 106, 218, 286, 396, 436, 520, 552, or 582, or to a reference sequence corresponding to SEQ ID NO: 4, 106, 218, 286, 396, 436, 520, 552, or 582, wherein the amino acid sequence comprises one or more substitutions relative to the reference sequence corresponding to residues 12 to 343 of SEQ ID NO: 4, 106, 218, 286, 396, 436, 520, 552, or 582, or relative to the reference sequence corresponding to SEQ ID NO: 4, 106, 218, 286, 396, 436, 520, 552, or 582.

20. The engineered RNA ligase of claim 1, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12 to 343 of an even-numbered SEQ ID NO of SEQ ID NOs: 4-220, 224-252, and 270-958, or to a reference sequence corresponding to an even-numbered SEQ ID NO of SEQ ID NOs: 4-220, 224-252, and 270-958, wherein the amino acid sequence comprises one or more substitutions relative to the reference sequence corresponding to residues 12 to 343 of SEQ ID NO: 4, 106, 218, 286, 396, 436, 520, 552, or 582, or relative to the reference sequence corresponding to SEQ ID NO: 4, 106, 218, 286, 396, 436, 520, 552, or 582.

21. The engineered RNA ligase of claim 19, wherein the amino acid sequence of the engineered RNA ligase comprises at least a substitution at amino acid position 11, 12, 14, 16, 17, 23, 25, 26, 30, 32, 33, 38, 40, 52, 57, 60, 68, 69, 71, 73, 75, 79, 82, 83, 92, 93, 95, 96, 98, 100, 101, 102, 103, 111, 114, 117, 118, 119, 120, 135, 136, 141, 142, 145, 147, 156, 167, 168, 170, 171, 174, 177, 179, 183, 184, 185, 188, 189, 190, 191, 192, 193, 196, 200, 202, 205, 206, 207, 209, 221, 229, 254, 255, 259, 270, 271, 287, 288, 289, 292, 295, 296, 300, 307, 320, 327, 332, 333, 334, 335, 336, 337, 339, 342, or 343, or combinations thereof, wherein the amino acid positions are relative to the reference sequence corresponding to residues 12 to 343 of SEQ ID NO: 4, 106, 218, 286, 396, 436, 520, 552, or 582, or relative to the reference sequence corresponding to SEQ ID NO: 4, 106, 218, 286, 396, 436, 520, 552, or 582.

22. The engineered RNA ligase of claim 19, wherein the amino acid sequence of the engineered RNA ligase comprises at least a substitution or amino acid residue 11P, 12S, 14F/R/T/W, 16R, 17P, 23M, 25A/G/K/L/W, 26F/G/H/L/P/R/S/V, 30A/C/H/R, 32R, 33A/K, 38D, 40E, 52L, 57T, 60R, 68W, 69L, 71K, 73T, 75L/Q/T, 79W, 82I, 83P, 92A/E/G/M/N/S, 93A, 95E/V, 96A/S, 98D/E/P/R/S/Y, 100R, 101G, 102T, 103I, 111L, 114F/G/K/N/P/R/S/V, 117A/C/F/G/H/I/K/L/M/R/S/T/V/W, 118G/L/Q, 119A/E/S/T, 120P, 135Q/T, 136A, 141A/C/E/T, 142H/R, 145D, 147L, 156C/F/Y, 167A, 168A, 170E, 171E, 174Q, 177I/L, 179F/L, 183V, 184L/M/P/R, 185H/S, 188S, 189L/V, 190F, 191K, 192V, 193N, 196K, 200S/T, 202W, 205R/V, 206R, 207S/T, 209E/L, 221E, 229A, 254G, 255E/S, 259V, 270A, 271I, 287A, 288A, 289A/H/K/S/T, 292E/Y, 295R, 296E/L/R/V/W, 300E/K, 307A, 320D/K, 327I/Q/R/W, 332R, 333A/E/S/T, 334S, 335E/H/K/L, 336S, 337G, 339K/R, 342I/T, 343P, 339K/R, or 342I/T, or combinations thereof, wherein the amino acid positions are relative to the reference sequence corresponding to residues 12 to 343 of SEQ ID NO: 4, 106, 218, 286, 396, 436, 520, 552, or 582, or relative to the reference sequence corresponding to SEQ ID NO: 4, 106, 218, 286, 396, 436, 520, 552, or 582.

23. The engineered RNA ligase of claim 19, wherein the amino acid sequence of the engineered RNA ligase comprises at least a substitution at amino acid position 33, 38, 71, 73, 75, 96, 98, 101, 114, 117, 136, 156, 179, 184, 191, 196, 221, 289, 292, 296, 320, 335, 336, or 339, or combinations thereof, wherein the amino acid positions are relative to the reference sequence corresponding to residues 12 to 343 of SEQ ID NO: 4, 106, 218, 286, 396, 436, 520, 552, or 582, or relative to the reference sequence corresponding to SEQ ID NO: 4, 106, 218, 286, 396, 436, 520, 552, or 582.

24. The engineered RNA ligase of claim 19, wherein the amino acid sequence of the engineered RNA ligase comprises at least a substitution or amino acid residue 33A/K, 38D, 71K, 73T, 75L/Q/T, 96A/S, 98D/E/P/R/S/Y, 101G, 114F/G/K/N/P/R/S/V, 117A/C/F/G/H/I/K/L/M/R/S/T/V/W, 136A, 156C/F/Y, 179F/L, 184L/M/P/R, 191K, 196K, 221E, 289A/H/K/S/T, 292E/Y, 296E/L/R/V/W, 320D/K, 335E/H/K/L, 336S, or 339K/R, or combinations thereof, wherein the amino acid positions are relative to the reference sequence corresponding to residues 12 to 343 of SEQ ID NO: 4, 106, 218, 286, 396, 436, 520, 552, or 582, or relative to the reference sequence corresponding to SEQ ID NO: 4, 106, 218, 286, 396, 436, 520, 552, or 582.

25-64. (canceled)

65. The engineered RNA ligase of claim 1, wherein the engineered RNA ligase comprises an amino acid sequence comprising residues 12 to 343 of an even numbered SEQ ID NO. of SEQ ID NOs: 4-220, 224-252, and 270-958, or an amino acid sequence comprising an even numbered SEQ ID NO. of SEQ ID NOs: 4-220, 224-252, and 270-958.

66-68. (canceled)

69. The engineered RNA ligase of claim 1, wherein the engineered RNA ligase has RNA ligase activity and an improved property, wherein the improved property is selected from i) increased activity, ii) increased product yield, iii) increased product yield on polynucleotides with phosphorothioate internucleotide linkages, iv) increased product yield on oligonucleotides with 2′-modifications, and v) increased expression, or any combination of i), ii), iii), iv), and v), compared to a reference RNA ligase having a sequence corresponding to SEQ ID NO: 2, 4, 106, 218, 286, 396, 436, 520, 552, or 582.

70-73. (canceled)

74. A recombinant polynucleotide encoding an engineered RNA ligase of claim 1.

75-79. (canceled)

80. An expression vector comprising a recombinant polynucleotide of claim 74.

81. (canceled)

82. (canceled)

83. A host cell comprising an expression vector of claim 80.

84. (canceled)

85. (canceled)

86. A method of producing an engineered RNA ligase polypeptide in a host cell comprising culturing a host cell of claim 83, under suitable culture conditions such that at least one engineered RNA ligase is produced.

87. (canceled)

88. (canceled)

89. A composition comprising an RNA ligase of claim 1.

90. (canceled)

91. A method of ligating at least a first polynucleotide strand and a second polynucleotide strand, comprising contacting a first polynucleotide strand and a second polynucleotide strand with an engineered RNA ligase of claim 1 in presence of a nucleotide substrate under conditions suitable for ligation of the first polynucleotide strand to the second polynucleotide strand, wherein the first polynucleotide strand comprises a ligatable 5′-end and the second polynucleotide strand comprises a 3′-end ligatable to the 5′-end of the first polynucleotide.

92-108. (canceled)

109. A kit comprising at least an RNA ligase of claim 1.

110. (canceled)
